(19) United States
(12) Patent Application Publication
(10) Pub. No.: US 2012/0238835 A1
(43) Pub. Date: Sep. 20, 2012

(54) System, devices, and methods for real-time monitoring of cerebrospinal fluid for markers of progressive conditions

(75) Inventors: Roderick A. Hyde, Redmond, WA (US); Sharon L. Wolda, Seattle, WA (US); Lowell L. Wood, JR., Bellevue, WA (US)

(73) Assignee: Searete LLC, a limited liability corporation of the State of Delaware

(21) Appl. No.: 13/065,342

(22) Filed: Mar. 17, 2011

Related U.S. Application Data
(63) Continuation of application No. 13/065,267, filed on Mar. 16, 2011.
Application No. 13/065,267 is a continuation of application No. 13/065,328, filed on Mar. 17, 2011.
Application No. 13/065,328 is a continuation of application No. 13/065,329, filed on Mar. 17, 2011.
Application No. 13/065,329 is a continuation of application No. 13/065,332, filed on Mar. 17, 2011.
Application No. 13/065,332 is a continuation of application No. 13/065,330, filed on Mar. 17, 2011.

(51) Int. Cl. A61B 5/07

(52) U.S. Cl. 600/302

(57) ABSTRACT

Systems, devices, methods, and compositions are described for providing real-time monitoring of cerebrospinal fluid for markers of progressive conditions.

### SUMMARY

In an aspect, the present disclosure is directed to, among other things, an indwelling implant including a biomarker detection circuit configured to detect a biomarker profile of cerebrospinal fluid (CSF) received within one or more fluid-flow passageways of a body structure configured to receive CSF. In an embodiment, the biomarker detection circuit includes a sensor having a biological molecule capture layer incorporating a plurality of targeting moieties for identifying one or more factors associated with a specific disease state, pathology, or condition. In an embodiment, the biomarker detection circuit includes at least one computing device operably coupled to an interrogation energy source and to a sensor that generates a response based on changes to a resonance condition of a plasmon-resonance-supporting portion. In an embodiment, the computing device generates a response indicative of the presence of one or more CSF biomarkers based on changes to a resonance condition of the plasmon-resonance-supporting portion.

In an embodiment, the indwelling implant includes a mental disorder biomarker identification circuit that compares a detected biomarker profile, acquired by the biomarker detection circuit, of CSF received within the one or more fluid-flow passageways to filtering information (e.g., mental disorder filtering information, reference biomarker identification information, etc.). In an embodiment, the mental disorder biomarker identification circuit includes one or more computing devices that access discrete data structures having user-specific filtering information stored thereon. For example, in an embodiment, the mental disorder biomarker identification circuit includes one or more computing devices that accesses at least one data structure having user-specific filtering information configured as a physical data structure. In an embodiment, the mental disorder biomarker identification circuit includes one or more data structures having at least one lookup table configured as shift registers and having data representative of user-specific filtering information. In an embodiment, the mental disorder biomarker identification circuit includes one or more data structures having at least one of mental disorder state marker information, mental disorder trait information, or heuristically determined mental disorder information stored thereon. In an embodiment, the mental disorder biomarker identification circuit includes one or more data structures having at least one of reference biomarker information (e.g., reference biomarker spectral response information, reference biomarker optical response information, or the like), reference neuropsychiatric disorder spectral information, or reference neurodegenerative disorder spectral information stored thereon.

In an aspect, the present disclosure is directed to, among other things, a method for monitoring CSF biomarkers indicative of suicidal tendencies. In an embodiment, the method includes detecting in vivo CSF spectral information of a biological subject indicative of a pathological change via one or more indwelling implants, at a plurality of sequential times. For example, in an embodiment, the method includes detecting in vivo CSF spectral information of a biological subject indicative of a change to a CSF serotonin metabolite level via one or more indwelling implants, at a plurality of sequential times. In an embodiment, the method includes in situ, real-time comparing, of detected in vivo CSF spectral information to user-specific spectral model information. In an embodiment, the method includes generating a suicidal tendency status.

In an aspect, the present disclosure is directed to a telematic monitoring implantable device including, among other things, a biomarker telematic information generation circuit configured to generate biomarker telematic information associated with at least one of an in vivo detected CSF neurological disorder biomarker or a CSF psychiatric disorder biomarker. In an embodiment, the telematic monitoring implantable device includes a telematic biomarker reporter circuit configured to transmit at least one of neurological disorder biomarker information or CSF psychiatric disorder biomarker information.

In an aspect, the present disclosure is directed to an implantable wireless biotelemetry device including, among other things, a sensor component configured to detect at least one biomarker profile of CSF received within one or more fluid-flow passageways of the implantable wireless biotelemetry device. In an embodiment, the implantable wireless biotelemetry device includes one or more computer-readable memory media including executable instructions stored thereon that, when executed on a computer, instruct a computing device to retrieve from storage one or more parameters associated with reference CSF biomarker spectral information associated with at least one neuropsychiatric disorder. In an embodiment, the implantable wireless biotelemetry device includes one or more computer-readable memory media including executable instructions stored thereon that, when executed on a computer, instruct a computing device to perform a comparison of a detected biomarker profile to a retrieved set of parameters. In an embodiment, the implantable wireless biotelemetry device includes a transceiver that concurrently or sequentially transmits or receives information in response to the comparison.

In an aspect, the present disclosure is directed to a system including, among other things, a CSF marker detection circuit configured to obtain in vivo CSF information of CSF proximate a surface of an indwelling shunt, and a decision signal circuit configured to signal a decision whether to transmit a notification in response to one or more comparisons between filtering information specific to the biological subject and obtained in vivo CSF information.

In an aspect, the present disclosure is directed to a system including, among other things, neuropsychiatric disorder information generation circuit configured to generate neuropsychiatric disorder biomarker information of CSF applied to an array, and a biomarker information comparison circuit configured to generate a comparison between the generated neuropsychiatric disorder biomarker information and user-specific filtering information. In an embodiment, the neuropsychiatric disorder information generation circuit is configured to generate neuropsychiatric disorder biomarker information of CSF applied to an array having capture regions that specifically binds to one or more biomarkers indicative of at least one of Alzheimer's disease, amyotrophic lateral sclerosis, bipolar disorder, Creutzfeldt-Jakob disease, dementia, depression, encephalitis, HIV associated dementia, ischemia, major depressive disorder, meningitis, multiple sclerosis, neuropsychiatric disorder, Parkinson's disease, psychosis, schizophrenia, sclerosis, suicidal tendency, or traumatic brain injury.

In an aspect, the present disclosure is directed to a real-time monitoring method including, among other things, obtaining in vivo CSF spectral information of a biological subject via an implanted sensor component. In an embodiment, the method includes determining whether to transmit a notification in response to one or more comparisons between filtering information specific to the biological subject and obtained in vivo CSF information of the biological subject.

In an aspect, the present disclosure is directed to an in vivo method for real-time monitoring of one or more biomarkers within CSF including, among other things, comparing, using integrated circuitry, a detected energy spectral profile of CSF proximate a surface of an indwelling implant to neuropsychiatric disorder spectral information configured as a physical data structure. In an embodiment, the detected energy spectral profile includes at least one of energy absorption spectral information, energy reflection spectral information, or energy transmission spectral information associated with one or more biomarkers within CSF. In an embodiment, the in vivo method for real-time monitoring includes generating a response based on the comparing of the detected energy spectral profile to the neuropsychiatric disorder spectral information.

In an aspect, the present disclosure is directed to an in vivo real-time monitoring method including determining relative change information from a comparison between one or more spectral components of at least a second in time detected energy spectral profile of CSF proximate a surface of an indwelling implant and one or more spectral components of a first in time detected energy spectral profile of CSF proximate the surface of the indwelling implant. In an embodiment, the method includes comparing the determined relative change information to reference neuropsychiatric disorder spectral component information stored in one or more non-transitory computer-readable memory media onboard the indwelling implant. In an embodiment, the detected neuropsychiatric disorder spectral component information includes at least one of CSF biomarker spectral information associated with a neuropsychiatric disorder prodrome or CSF biomarker spectral information associated with a neuropsychiatric disorder.

In an aspect, the present disclosure is directed to a method for predicting an onset of a depressive disorder including transcutaneously communicating a suicidal tendency status in response to an in vivo comparison of CSF neuropeptide spectral information to reference filtering information.

In an aspect, the present disclosure is directed to a method for monitoring a pathological condition associated with a suicidal tendency including, among other things, real-time detecting, via an implanted shunt, one or more spectral components associated with at least one CSF cholecystokinin peptide. In an embodiment, the method for monitoring the pathological condition associated with a suicidal tendency includes generating at least one of an anxiety report, a depression status report, or a suicidal tendency report in response to spectral information associated with the real-time detected one or more spectral components associated with the at least one CSF cholecystokinin peptide.

In an aspect, the present disclosure is directed to a method including, among other things, comparing a sensor component output signal associated with CSF received within an indwelling implant and applied to a composition detector to user-specific filtering information. In an embodiment, the method includes generating a neuropsychiatric disorder assessment in response to the comparison.

In an aspect, the present disclosure is directed to an in vivo method for real-time monitoring of one or more biomarkers within CSF including, among other things, comparing a compositional multiplexed output associated with one or more biomarkers present in CSF received with an indwelling implant to user-specific neuropsychiatric disorder information configured as a physical data structure. In an embodiment, the method includes generating a response based on the comparing of the compositional multiplexed output to the user-specific neuropsychiatric disorder information.

In an aspect, the present disclosure is directed to a method for diagnosing schizophrenia including, among other things, detecting, via an indwelling sensor component, time series information associated with CSF proximate a surface of the indwelling implant and exposed to a panel of markers. In an embodiment, the method includes generating a real-time comparison between the detected time series information and user-specific schizophrenia prodromal marker information or user-specific schizophrenia marker information.

In an aspect, the present disclosure is directed to a method including, among other things, detecting, in vivo, a spectral profile of one or more CSF measurands obtained at a plurality of sequential time points from CSF received within an indwelling implant. In an embodiment, the method includes partitioning the detected energy spectral profile into one or more information subsets. In an embodiment, the method includes performing a real-time comparison of at least one of the one or more information subsets to reference neuropsychiatric disorder compositional information (e.g., user-specific neuropsychiatric disorder compositional information, heuristic neuropsychiatric disorder compositional information, modeled neuropsychiatric disorder compositional information, or the like).

In an aspect, the present disclosure is directed to a method including, among other things, executing at least one of a Spectral Clustering protocol or a Spectral Learning protocol operable to compare one or more parameters from an in vivo detected energy spectral profile associated with at least one CSF component, obtained at a plurality of sequential time points from CSF received within an indwelling implant, to one or more information subsets associated with reference neuropsychiatric disorder spectral information.

In an aspect, the present disclosure is directed to a method including, among other things, performing a real-time comparison of a first detected electromagnetic energy absorption profile of a first portion of CSF proximate an indwelling implant sensor to characteristic CSF spectral information. In an embodiment, the method includes determining whether a neuropsychiatric disorder status change has occurred. In an embodiment, the method includes obtaining a second detected electromagnetic energy absorption profile of a second portion of CSF proximate an indwelling implant sensor. In an embodiment, the method includes performing a real-time comparison of the second detected optical energy absorption profile to the characteristic CSF spectral information. In an embodiment, the method includes determining whether a neuropsychiatric disorder status change has occurred.

In an aspect, the present disclosure is directed to a method including, among other things, comparing an in vivo real-time detected measurand of CSF from an indwelling implant to biological subject specific filtering information configured as a physical data structure and stored in one or more non-transitory computer-readable memory media. In an embodiment, the method includes generating a response based at least in part on the generated one or more comparisons.

In an aspect, the present disclosure is directed to an in vivo method for real-time monitoring of one or more biomarkers within CSF including, among other things, performing an in vivo comparison of a detected change in a spectral absorption profile of one or more biomarkers present in CSF received with an implanted shunt to neuropsychiatric disorder information. In an embodiment, the method includes transcutaneously transmitting a response based on the comparison of the detected energy spectral profile to the characteristic spectral signature information.

In an aspect, a monitoring method includes generating one or more comparisons between at least one in vivo real-time detected measurand from an indwelling implant and biological subject specific filtering information configured as a physical data structure and stored in one or more non-transitory computer-readable memory media carried by the indwelling implant. In an embodiment, the method includes generating a response based at least in part on the generated one or more comparisons.

In an aspect, a real-time in vivo method of assessing a treatment efficacy or a treatment compliance associated with an acute or a chronic neuropsychiatric condition includes determining a compliance status of a user in response to spectral information obtained at a plurality of time points, the spectral information including one or more spectral components associated with a compliance marker within CSF. In an embodiment, the method includes generating a response indicative of a compliance status.

In an aspect, a telematic monitoring method includes generating biomarker telematic information associated with at least one in vivo detected CSF neurological disorder biomarker or CSF psychiatric disorder biomarker. In an embodiment, the method includes transmitting at least one of neurological disorder biomarker information or CSF psychiatric disorder biomarker information.

In an aspect, an in vivo method for real-time monitoring of one or more biomarkers within CSF includes comparing, using integrated circuitry, a detected energy spectral profile of CSF proximate a surface of an indwelling implant to neuropsychiatric disorder spectral information configured as a physical data structure. In an embodiment, the detected energy spectral profile includes at least one of energy absorption spectral information, energy reflection spectral information, or energy transmission spectral information associated with one or more biomarkers within CSF. In an embodiment, the method includes generating a response based on the comparing of the detected energy spectral profile to the neuropsychiatric disorder spectral information.

In an aspect, the present disclosure is directed to a method including, among other things, detecting, in vivo, a spectral profile of one or more CSF measurands obtained at a plurality of sequential time points from CSF received within an indwelling implant. In an embodiment, the method includes partitioning the detected energy spectral profile into one or more information subsets. In an embodiment, the method includes performing a real-time comparison of at least one of the one or more information subsets to user-specific neuropsychiatric disorder spectral information.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1A is a perspective view of a system including an implantable device according to one embodiment.

FIG. 1B is a perspective view of a system including an implantable device according to one embodiment.

FIG. 2 is a perspective view of a system including an implantable device according to one embodiment

FIG. 3A is a perspective view of a system including an implantable device according to one embodiment.

FIG. 3B is a perspective view of a system including an implantable device according to one embodiment.

FIG. 4A is a perspective view of a system including an implantable device according to one embodiment.

FIG. 4B is a perspective view of a system including an implantable device according to one embodiment.

FIG. 4C is a perspective view of a system including a telematic monitoring implantable device according to one embodiment.

FIGS. 5A, 5B, and 5C show a flow diagram of a method according to one embodiment.

FIG. 6 is a flow diagram of a method according to one embodiment.

FIGS. 7A, 7B, and 7C show a flow diagram of a method according to one embodiment.

FIG. 8 is a flow diagram of a method according to one embodiment.

FIGS. 9A, 9B, and 9C show a flow diagram of a method according to one embodiment.

FIGS. 10A, 10B, and 10C show a flow diagram of a method according to one embodiment.

FIGS. 11A and 11B show a flow diagram of a method according to one embodiment.

FIG. 12 is a flow diagram of a method according to one embodiment.

FIGS. 13A and 13B show a flow diagram of a method according to one embodiment.

FIG. 14 is a flow diagram of a method according to one embodiment.

FIGS. 15A and 15B show a flow diagram of a method according to one embodiment.

FIG. 16 is a flow diagram of a method according to one embodiment.

FIGS. 17A and 17B show a flow diagram of a method according to one embodiment.

FIG. 18 is a flow diagram of a method according to one embodiment.

FIG. 19 is a flow diagram of a method according to one embodiment.

FIG. 20 is a flow diagram of a method according to one embodiment.

FIG. 21 is a flow diagram of a method according to one embodiment.

FIGS. 22A and 22B show a flow diagram of a method according to one embodiment.

FIG. 23 is a flow diagram of a method according to one embodiment.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments can be utilized, and other changes can be made, without departing from the spirit or scope of the subject matter presented here.

Mental, behavioral, and neurological disorders represent a significant portion of the global disease burden affecting approximately 450 million globally. See, e.g., World Health Organization, Investing in Mental Health, WHO: Geneva (2003). Estimates made by World Health Organization in 2009 showed that globally about 151 million people suffer from depression; 26 million people from schizophrenia, 24 million from Alzheimer and other dementias, and 18 million from neuroinfections or neurological sequelae of infections. See Michelle Funk et al., Mental Health and Development: Targeting People with Mental Health Conditions as a Vulnerable, World Health Organization (2010), (available at: http://whqlibdoc.who.int/publications/2010/9789241563949_eng_full.pdf; accessed 30 Sep. 2010). The World Health Organization notes that one in four patients visiting a health service has at least one mental, neurological, or behavioral disorder; most remaining undiagnosed and untreated. On average, approximately 844,000 commit suicide every year. Id.

Real-time monitoring of markers (e.g., biomarkers, chemicals, components, materials, substances, or the like) within CSF for neuropsychiatric disorders (e.g., anxiety disorder, bipolar disorder, depression, affective disorder, mood spectrum disorder, schizophrenia, etc.), neurodegenerative disorder (e.g., Alzheimer's Disease, Huntington's disease, Parkinson's disease, other dementias, etc.), or the like can facilitate disease diagnosis, improve tracking of disease progression, as well as help to evaluate the efficacy or compliance of current and future treatments. For example, early identification and intervention can improve prognosis. Likewise, in vivo multiplex real-time monitoring of multiple biological markers can allow for earlier diagnosis, as well as improve treatment and prognosis.

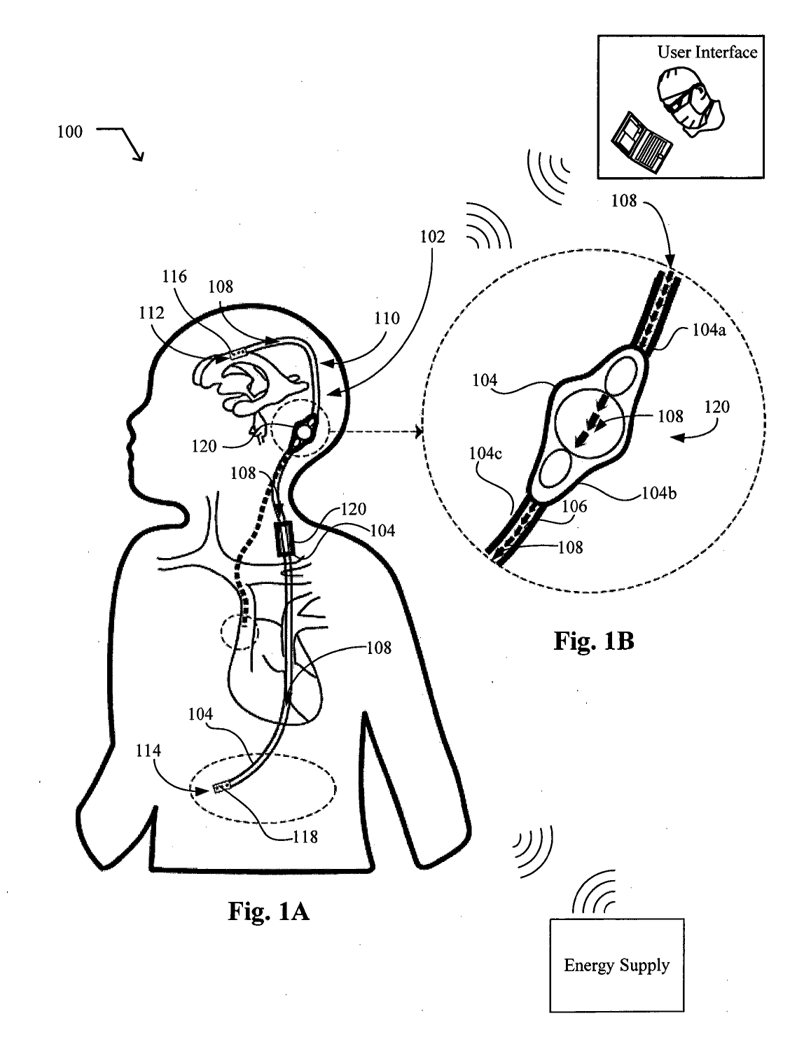

FIGS. 1A and 1B show a system 100 (e.g., a shunt system, a catheter system, an implantable system, an implantable shunt system, an implantable sensor system, an implantable catheter system, a partially implantable system, or the like) in which one or more methodologies or technologies can be implemented such as, for example, managing a transport of biological fluids and actively detecting, diagnosing, preventing, or treating mental disorders (e.g., neuropsychiatric disorders, neurodegenerative disorders, or the like), disease states, pathological conditions, or the like. In an embodiment, the system 100 includes, among other things, one or more implantable devices 102. An implantable device 102 can be configured to, among other things, have numerous configurations. For example, in an embodiment, the system 100 includes partially or completely implantable devices 102 or components that are partially or completely implantable.

Non-limiting examples of implantable devices 102 include shunts (e.g., cardiac shunts, cerebral shunts, cerebrospinal fluid shunts (an example of which is shown on FIG. 1A); lumbo-peritoneal shunts; portacaval shunts; portosystemic shunts, pulmonary shunts, or the like); catheters (e.g., central venous catheters, multi-lumen catheters, peripherally inserted central catheters, Quinton catheters, Swan-Ganz catheters, tunneled catheters, urinary catheters, vascular catheters, or the like); medical ports (e.g., arterial ports, low profile ports, multi-lumen ports, vascular ports, or the like); indwelling sensors (e.g., subcutaneous sensors); telematic monitoring devices; or the like. Further non-limiting examples of implantable devices 102 include bio-implants, bioactive implants, indwelling implants, indwelling sensors, implantable electronic device, implantable medical devices, or the like. Further non-limiting examples of implantable devices 102 include replacements implants (e.g., joint replacements implants such, for example, elbows, hip, knee, shoulder, wrists replacements implants, or the like); subcutaneous drug delivery devices (e.g., implantable pills, drug-eluting stents, or the like); stents (e.g., coronary stents, peripheral vascular stents, prostatic stents, ureteral stents, vascular stents, or the like); biological fluid flow controlling implants; or the like. Further non-limiting examples of implantable devices 102 include artificial hearts, artificial joints, artificial prosthetics, contact lens, mechanical heart valves, subcutaneous sensors, or the like.

In an embodiment, the implantable device 102 includes one or more biocompatible materials, polymeric materials, thermoplastics, silicone materials (e.g., polydimethysiloxanes), polyvinyl chloride materials, latex rubber materials, or the like. Non-limiting examples of catheters or shunts, or components thereof can be found in, for example the following documents: U.S. Pat. No. 7,524,298 (issued Apr. 28, 2009), U.S. Pat. No. 7,390,310 (issued Jun. 24, 2008), U.S. Pat. No. 7,334,594 (issued Feb. 26, 2008), U.S. Pat. No. 7,309,330 (issued Dec. 18, 2007), U.S. Pat. No. 7,226,441 (issued Jun. 5, 2007), U.S. Pat. No. 7,118,548 (issued Oct. 10, 2006), U.S. Pat. No. 6,932,787 (issued Aug. 23, 2005), U.S. Pat. No. 6,913,589 (issued Jul. 5, 2005), U.S. Pat. No. 6,743,190 (issued Jun. 1, 2004), U.S. Pat. No. 6,585,677 (issued Jul. 1, 2003); and U.S. Patent Publication Nos. 2009/0118661 (published May 7, 2009), 2009/0054824 (published Feb. 26, 2009), 2009/0054827 (published Feb. 26, 2009), 2008/0039768 (published Feb. 14, 2008), 2006/0004317 (published Jan. 5, 2006); each of which is incorporated herein by reference.

In an embodiment, the implantable device 102 includes, among other things, a body structure 104 having at least one inner surface 106 defining one or more fluid-flow passageways 108 configured to receive cerebrospinal fluid (CSF). The one or more fluid-flow passageways 108 can take a variety of shapes, configurations, and geometric forms including regular or irregular forms and can have a cross-section of substantially any shape including, among others, circular, triangular, square, rectangular, polygonal, regular or irregular shapes, or the like, as well as other symmetrical and asymmetrical shapes, or combinations thereof. In an embodiment, the body structure 104 includes one or more compartments configured to receive CSF.

In an embodiment, one or more portions of the body structure 104 take a substantially cylindrical geometric form (e.g., a tubular structure) having an inner surface 106 defining one or more fluid-flow passageways 108. The substantially cylindrical geometric form can have a cross-section of substantially any shape including, among others, circular, triangular, square, rectangular, polygonal, regular or irregular shapes, or the like, as well as other symmetrical and asymmetrical shapes, or combinations thereof. In an embodiment, the substantially cylindrical geometric form includes multi-lumen structures (e.g., multi-lumen tubing) having multiple fluid-flow passageways 108 running therethrough. In an embodiment, the body structure 104 includes one or more tubular structures (e.g., multilayer tubular structures, tubular catheter body structures, tubular shunt body structures, multi-lumen tubular structures, or the like) defining one or more fluid-flow passageways 108. In an embodiment, the implantable device 102 includes one or more fluid-flow passageways 108 for receiving CSF of a biological subject.

In an embodiment, the body structure 104 includes a plurality of connected segments 104a, 104b, 104c. In an embodiment, the body structure 104 includes a plurality of segments 104a, 104b, 104c coupled along a longitudinal length. In an embodiment, the body structure 104 includes a plurality of segments 104a, 104b, 104c in fluid communication. In an embodiment, the body structure 104 includes a plurality of segments 104a, 104b, 104c connected via separate components. In an embodiment, the body structure 104 is configured as a monolithic structure. In an embodiment, the body structure 104 comprises an integrally formed component assembly. In an embodiment, the body structure 104 includes a plurality of segments configured in fluid communication 104a, 104b, 104c that are operable to transport a biological fluid.

In an embodiment, the implantable device 102 includes a body structure 104 having one or more shunts 110. In an embodiment, the implantable device 102 includes one or more shunts 110 configured to manage the transport of a body fluid (e.g., cerebrospinal fluid) from one region within the body (e.g. cerebral ventricle, lumbar sub-arachnoid spaces, or the like) to another (e.g., right atrium of the heart, peritoneal cavity, or the like). In an embodiment, the implantable device 102 includes a body structure 104 having one or more shunts 110 each including a proximal portion 112, a distal portion 114, and at least one inner fluid-flow passageway 108 extending therethrough.

Non-limiting examples of shunts 110 include blalock-taussig shunts, cardiac shunts, cerebral shunts (e.g., cerebrospinal fluid shunts, ventriculo-atrial shunts, ventriculo-peritoneal shunts, or the like) glaucoma shunts, Lumbar shunts (e.g., Lumbar-peritoneal shunts, Lumbar subcutaneous shunts, or the like) mechanical shunts, pulmonary shunts, portosystemic shunts, portoacaval shunts, ventricle-to-pulmonary artery conduits, or the like. Further non-limiting examples of shunts 110 can be found in, for example the following documents: U.S. Patent Publication Nos. 2008/0039768 (published Feb. 14, 2008) and 2006/0004317 (published Jan. 5, 2006); each of which is incorporated herein by reference.

In an embodiment, one or more of the shunts 110 are configured to regulate a pressure or flow of fluid (e.g., cerebrospinal fluid) from the ventricles. For example, an implantable device 102 including one or more shunts 110 can be useful to manage a CSF transport associated with hydrocephalus (a condition including enlarged ventricles). In hydrocephalus, pressure from CSF generally increases. Hydrocephalus develops when CSF cannot flow through the ventricular system, or when absorption into the blood stream is not the same as the amount of CSF produced. Indicators for hydrocephalus include headache, personality disturbances, loss of intellectual abilities (dementia), problems in walking, irritability, vomiting, abnormal eye movements, a low level of consciousness, or the like. Normal pressure hydrocephalus is associated with progressive dementia, problems in walking, and loss of bladder control (urinary incontinence).

The implantable device 102 is configured to, among other things, manage a transport of biological fluids. In an embodiment, the implantable device 102 includes, among other things, one or more ports configured to provide access from, or to, an interior environment of at least one of the one or more fluid-flow passageways 108. In an embodiment, the implantable device 102 includes one or more fluid entry ports 116 and fluid exit ports 118 in fluid communication with an interior environment of at least one of the one or more fluid-flow passageways 108 to an exterior environment. In an embodiment, the implantable device 102 includes, among other things, one or more fluid entry ports 116 configured to provide fluidic access to an interior of at least one of the one or more fluid-flow passageways 108. In an embodiment, the implantable device 102 includes, among other things, one or more fluid exit ports 118 configured to provide fluidic access to an exterior of at least one of the one or more fluid-flow passageways 108. In an embodiment, the implantable device 102 includes one or more cannulas configured to drain CSF from a ventricle of a brain of the biological subject. In an embodiment, the implantable device 102 includes one or more ventriculoperitoneal shunts.

In an embodiment, the implantable device 102 includes one or more CSF shunts configured to drain CSF from a region of a brain of the biological subject. In an embodiment, the CSF shunt includes entry conduits, such as a proximal (ventricular) catheter, into cranium and lateral ventricle, subcutaneous conduits, such as a distal catheter, and one or more flow-regulating devices for regulating flow of fluid out of the brain and into a peritoneal cavity.

In an embodiment, the implantable device 102 is configured to bypass malfunctioning arachnoidal granulations and to drain an excess fluid from the cerebral ventricles into one or more internal delivery regions (e.g., peritoneal cavity, pleural cavity, right atrium, gallbladder, or the like). For example, an implantable device 102 including one or more shunts 110 is surgically implanted to provide a controllable fluid-flow passageway 108 that diverts CSF away from central nervous system fluid compartments (e.g., ventricles, fluid spaces near the spine, or the like) to one or more internal delivery regions including, for example, the peritoneal cavity (ventriculo-peritoneal shunt), the pleural cavity (ventriculo-pleural shunt), the right atrium (ventriculo-atrial shunt), or the gallbladder.

In an embodiment, the implantable device 102 includes one or more flow-regulating devices 120. In an embodiment, the implantable device 102 includes one or more flow-regulating devices 120 within at least one fluid-flow passageway 108. In an embodiment, the one or more flow-regulating devices 120 include at least one valve assemblies having one or more of a housing, inlet and outlet ports, fluid-flow passageways 108, adjustable pressure valves, mono-pressure valves, mechanical valves, electro-mechanical valves, programmable valves, one-way valves, two-way valves, pulsar valves, shunt valves, electro-mechanical valve actuators, valve mechanisms (e.g., ball-in-cone mechanism, controllable diaphragms, valve diaphragms, or the like), valve seats, pressure control valves, shunt valves, flow restriction devices, flow control devices, shunts, catheters, or the like. In an embodiment, the implantable device, 102 includes one or more pressure (e.g., intracranial pressure) regulating devices 120. In an embodiment, the implantable device 102 includes a pressure-regulated valve means positioned within at least one fluid-flow passageway 108 for providing fluid flow therethrough at selected fluid pressures. Non-limiting examples of flow-regulating devices 120 include adjustable pressure valves, mono-pressure valves, mechanical valves, electro-mechanical valves, programmable valves, pulsar valves, catheter valves, shunt valves, or the like. Further non-limiting examples of flow-regulating devices 120 include differential pressure valves, one-way valves, flow-regulating or restricting valves, fixed pressure valves, (e.g., DELTA valves by Medtronic Neurological and Spinal), adjustable pressure valves (PS MEDICAL STRATA and STRATA valves by Medtronic Neurological and Spinal), CSF-flow control valves (Medtronic Neurological and Spinal).

In an embodiment, the implantable device 102 is configured to regulate a transport of material into or out of a biological subject. For example, in an embodiment, the implantable device 102 includes one or more flow-regulating devices 120 for regulating a transport of material into or out of a biological subject. In an embodiment, the implantable device 102 is configured to regulate a transport of material within a biological subject. In an embodiment, the implantable device 102 is configured to regulate fluidic flow in or out of a biological subject. In an embodiment, the implantable device 102 is configured to regulate fluidic flow from at least a first location of the body to at least a second location of the body. In an embodiment, the implantable device 102 is configured to regulate fluidic flow of CSF from a ventricle of the brain or a lumbar region, to a drainage location in the body.

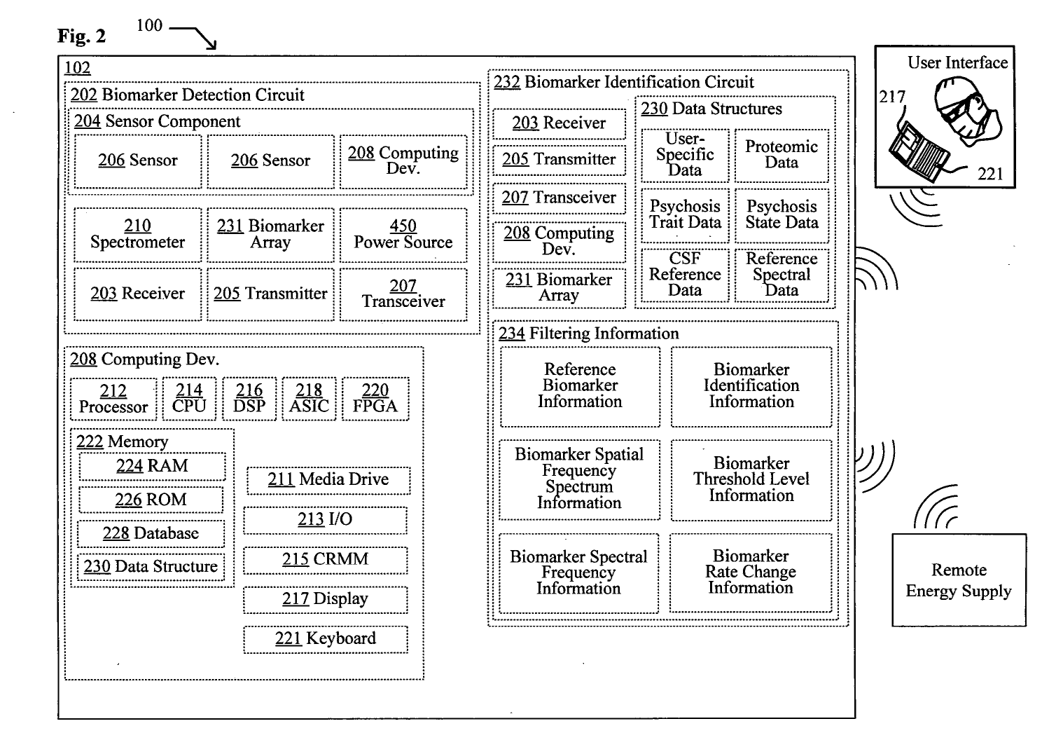

Referring to FIG. 2, in an embodiment, the system 100 includes, among other things, at least one implantable device 102 including a biomarker detection circuit 202. In an embodiment, the biomarker detection circuit 202 acquires at least one biomarker profile of CSF received within, or proximate to, an implantable device 102. For example, in an embodiment, the biomarker detection circuit 202 includes one or more sensor components 204 operable to detect (e.g., assess, calculate, evaluate, determine, gauge, measure, monitor, quantify, resolve, sense, or the like) at least one characteristic (e.g., a spectral characteristic, a spectral signature, a physical quantity, an environmental attribute, a physiologic characteristic, a response associated with a focal volume interrogated by an electromagnetic energy stimulus, or the like) associated with a biological sample (e.g., tissue, biological fluid, biomarker composition, infections agent composition, or the like) and indicative of a mental disorder, a disease state, a pathological condition, or the like. In an embodiment, the biomarker detection circuit 202 includes at least one sensor component 204 configured to detect (e.g., optically detect, acoustically detect, thermally detect, energetically detect, spectroscopically detect, or the like) one or more markers within CSF that are associated a mental disorder, a disease state, a pathological condition, or the like. In an embodiment, biomarker detection provides an objective measure of a biological or pathological process to evaluate disease risk or prognosis, to guide clinical diagnosis, or to monitor therapeutic interventions.

Cerebrospinal fluid is in direct contact with the extracellular space of the brain and as such can reflect biochemical changes that occur in association with a neuropsychiatric disorder. For example, relative changes in the concentration or level of one or more biomarkers in CSF can be indicative of a disease state, pathological condition, or the like. In an embodiment, the biomarker detection circuit 202 monitors at least one of materials, substances, chemicals, components, target biomarkers, or the like indicative of mental disorders, disease states, pathological conditions, or the like.

Non-limiting examples of disease states and pathological conditions of the central nervous system include autoimmune diseases and inflammatory diseases (e.g., multiple sclerosis, arachnoiditis, myelitis, Schilder's disease); tumors (e.g., gliomas, meningiomas, pituitary adenomas, vestibular schwannomas, primitive neuroectodermal tumors (medulloblastomas), as well as metastatic cancer); metastatic brain tumor (e.g., metastasis of melanoma, breast cancer, renal cell carcinoma, colorectal cancer); mental disorders (e.g., psychosis, schizophrenia, bipolar disorder, addiction, depression); anxiety disorders (e.g., generalized anxiety disorder, panic disorder, agoraphobia, phobias, social anxiety disorder, obsessive-compulsive disorder, post-traumatic stress disorder, separation anxiety); neurodegenerative (e.g., amyotrophic lateral sclerosis (ALS), Alzheimer's disease, Lewy body dementia, Parkinson's disease, Huntington's disease, corticobasal degeneration, frontotemporal dementia (Pick's disease), pantothenate kinase-associated neurodegeneration, Alper's disease, multiple system atrophy); cerebrovascular diseases (e.g., ischemic stroke, hemorrhagic stroke, brain ischemia); infections (meningitis, poliomyelitis, human immunodeficiency virus (HIV) associated dementia, encephalitis, encephalomyelitis, Herpes Simplex, syphilis, uveomeningoencephalitic syndrome, corticobasal ganglionic degeneration dementia encephalitis, neuroborreliosis, cerebral cysticercosis, trichinosis); prion diseases (Creutzfeldt-Jakob disease, Gerstmann-Straussler-Scheinker disease, kuru, fatal familial insomnia); leukodystrophies or lipid storage diseases (e.g., adrenoleukodystrophy, metachromatic leukodystrophy, Krabbe disease, Pelizaeus-Merzbacher disease, Canavan disease, Alexander disease, Refsum disease, Sandhoff disease, Niemann-Pick disease); ataxia disorders (e.g., spinocerebellar ataxia, ataxia telangiectasia, Machado-Joseph disease (MJD), as well as other neurological disorders (e.g., epilepsy, narcolepsy, Gilles de la Tourette's syndrome, Batten disease, progressive supranuclear palsy).

In an embodiment, the biomarker detection circuit 202 acquires spectral information associated with one or more mental disorders by detecting spectral differences between a first and a second region of the biological subject. Such “differential” measurements may allow for better signal to noise ratio, and may minimize the effect of other spectral parameters of the body that vary over time. In an embodiment, the biomarker detection circuit 202 is configured as a “differential mode” spectrometer. For example, in an embodiment, the biomarker detection circuit 202 detects spectral information associated with a biological sample at two or more time intervals. In an embodiment, during operation, the biomarker detection circuit 202 compares detected spectral information from at least one time interval to model spectral information or spectral information detected at a different time interval, and generates a response based on the comparison. In an embodiment, the biomarker detection circuit 202 concurrently or sequentially detects spectral information from multiple biomarkers at multiple locations within the biological subject.

In an embodiment, the biomarker detection circuit 202 includes one or more computing devices that are operable to isolate CSF spectral information, for example, by subtracting spectral information associated with one or more different tissues. In an embodiment, the biomarker detection circuit 202 isolates CSF spectral information by subtracting bone spectral information, fat spectral information, muscle spectral information, or the like, or other tissue spectral information. In an embodiment, the biomarker detection circuit 202 isolates CSF spectral information by subtracting spectral information associated with an implantable device. In an embodiment, the biomarker detection circuit 202 isolates CSF spectral information by subtracting user-specific information (e.g., user-specific model information).

In an embodiment, the biomarker detection circuit 202 is configured to acquire at least one biomarker profile of CSF received within one or more fluid-flow passageways 108 of the implantable device 102. For example, in an embodiment, the biomarker detection circuit 202 monitors one or more imaging probes associated with at least one of a cerebral spinal fluid marker.

Non-limiting examples of biomarkers include biomarkers associated with a specific disease state, pathological condition, or mental disorder, such as, for example, disease states and pathological conditions of the central nervous system. Non-limiting examples of biomarkers include immunoglobulins (e.g., oligoclonal IgG, kappa-free light chain immunoglobulin, immunoglobulin M, autoantibodies); amino acids and derivatives thereof (e.g., D-serine, gamma-aminobutyric acid (GABA), glutamine, glutamate, glycine, kynurenic acid); soluble receptors (e.g., soluble human leukocyte antigen G5 (sHLA-G1); soluble human leukocyte antigen G5 (sHLA-G5), soluble triggering receptor expressed on myeloid cells 2 (sTREM-2), neural cell adhesion molecule (NCAM)); peptide growth factors and peptide hormones (e.g., brain-derived neurotrophic factor (BDNF), fibroblast growth factor 2 (FGF-2), nerve growth factor (NGF), tissue growth factor beta 2 (TGF-beta2), tumor necrosis factor (TNF), vascular endothelial growth factor (VEGF), corticotrophin-releasing hormone, somatostatin, thyrotropin-releasing hormone); neurotransmitters (e.g., anandamide, norepinephrine, phenylethylamine, neuropeptide Y, orexin A); nucleotide derivatives (e.g., 8-hydroxyl-2-deoxyguanosine, 8-hydroxylguanosine, neopterin,); sugars (e.g., fructose, glucose, sorbitol); brain- and neuron-enriched proteins (e.g., 14-3-3 protein, glial fibrillary acid protein (GFAP), myelin basic protein (MBP), neurofilament (NFL), neuron-specific enolase (NSE), S100 calcium binding protein B (S100B), or synaptosome-associated protein of 25000 dalton (SNAP-25).

Further non-limiting examples of biomarkers include tau and phosphorylated tau, amyloid beta 42); enzymes (e.g., alanine aminotransferase, angiotensin converting enzyme, beta-glucuronidase, creatine kinase BB, glycogen synthase kinase 3 beta (GSK3beta), glucose 6-phosphate isomerase, hexosaminidase A (HexA), inositol monophosphatase, lactase dehydrogenase, urokinase (uPA)); inflammatory mediators (e.g., beta 2 microglobulin, chemokine (C-C motif) ligand 5 (CCR5), chemokine (C-X-C motif) ligand 10 (CXCL10), interferon gamma (IFN-gamma), interleukin 6 (IL-6), interleukin 8 (IL-8), interleukin 17 (IL-17), monocyte chemotactic protein (MCP)); other small proteins and peptides (e.g., 7B2-carboxy terminus, angiotensin II, cystatin C, cystatin C fragment, cytochrome C, peptide YY, PINCH, transferrin, transthyretin, ubiquitin, Vgf); fatty acids, lipids and lipid-associated proteins (e.g., docosohexanoic acid, ceramide, 4-hydroxynonenals (HNE), apolipoprotein A1, apolipoprotein J); secreted glycoproteins (e.g., chromogranin A, chromogranin B, secretogranin II); other small molecules (e.g., bilirubin, heme, 5-hydroxyindoleacetic acid (5-HIAA), homovanillic acid (HVA), lactate, nitrate, nitric oxide, nitrite); cells (e.g., CD4+ lymphocytes, neutrophils, leukemic cells); pathogen DNA; or pathogen RNA.

Further non-limiting examples of biomarkers include CSF components, blood components, or the like. Non-limiting examples of CSF components include electrolytes (e.g., to sodium, potassium, chloride, carbon dioxide, calcium, magnesium, lactate, or the like) and other small molecular components, proteins, or a limited number of cells.

Non-limiting examples of biomarkers in CSF include basic biomarkers (e.g., those used to identify conditions that might mimic or coexist with a neuropsychiatric disorder) or core biomarkers (e.g., those used to identify the pathogenic process of a neuropsychiatric disorder). Non-limiting examples of basic biomarkers include biomarkers of blood-brain barrier integrity and/or inflammatory processes, which may not be specific for a given neuropsychiatric disorder. For example, the level of albumin in CSF relative to the level of albumin in the serum (the albumin quotient (QA); CSF_{alb}/serum_{alb}) provides a simple measure of the integrity of the blood brain barrier. In an embodiment, normal blood brain barrier permeability in adults is defined as a QA≦0.007, and a damaged or open blood brain barrier is defined as mild (QA=0.007−0.01), moderate (QA=0.01−0.02), and severe (QA≧0.02), respectively. (See, e.g., Blyth, et al., J. Neurotrauma, 26:1497-1507, (2009); which is incorporated herein by reference).

Non-limiting examples of core biomarkers include biomarkers coupled to the underlying molecular pathology of a disease. In Alzheimer's disease, for example, core biomarkers reflect amyloid and neurofibrillary tangle pathology and axonal degeneration. In addition, the biomarkers can be indicative of cellular damage to one or more components of the central nervous system. Non-limiting examples of biomarkers with a cellular origin include biomarkers derived from neurons (e.g., neuron-specific enolase (NSE), 14-3-3, tau protein, amyloid precursor protein, amyloid peptides (e.g., Aβ42), neurofilament proteins, and chromagrannins A and B); biomarkers derived from astrocytes (e.g., S-100β and glial acid fibrillary proteins); biomarkers derived from leptomeninges (e.g., β-trace protein and cystatin C); biomarkers derived from microglial cells (e.g., ferritin); biomarkers derived from oligodendrocytes (e.g., myelin basic protein, proteolipid protein, and myelin oligodendrocytic glycoprotein); or biomarkers derived from the choroid plexus (e.g., transthyretin). (See, e.g., Green, Neuropath. Appl. Neurobiol., 28:427-440, (2002), Blennow, et al., Nat. Rev. Neurol., 6:131-144, 2010; each of which is incorporated herein by reference).

Non-limiting examples of proteins in CSF include albumin, prostaglandin D-synthase, immunoglobulin G, transthyretin, transferrin, alpha1-antitrypsin, apolipoprotein, cystatin-C, alpha-1 acid glycoprotein, hemopexin. In an embodiment, a detected increase in total central nervous system (CNS) protein above 1 gm/liter can be indicative of a disease state and/or pathological condition such as, for example, inflammation, tumors, demyelinating disorders, orsubarachnoid hemorrhage and can arise from increased release of proteins from the CNS and/or a loss of integrity of the blood-brain barrier. In contrast, a decrease in total CSF proteins can be associated with water intoxication, leukemia, CSF leakage, rhinorrhea, otorrhea, hyperthyroidism, or pneumoencephalography. Cerebrospinal fluid also contains a small number of monocytes and/or lymphocytes with a total cell count less than 5 cells per cubic millimeter. In an embodiment, the presence of polymorphonuclear leukocytes (e.g., neutrophils) in CSF is indicative of infection or inflammatory response.

Non-limiting examples of detectable blood components include erythrocytes, leukocytes (e.g., basophils, granulocytes, eosinophils, monocytes, macrophages, lymphocytes, neutrophils, or the like), thrombocytes, acetoacetate, acetone, acetylcholine, adenosine triphosphate, adrenocorticotrophic hormone, alanine, albumin, aldosterone, aluminum, amyloid proteins (non-immunoglobulin), antibodies, apolipoproteins, ascorbic acid, aspartic acid, bicarbonate, bile acids, bilirubin, biotin, blood urea nitrogen, bradykinin, bromide, cadmium, calciferol, calcitonin (ct), calcium, carbon dioxide, carboxyhemoglobin (as HbcO), cell-related plasma proteins, cholecystokinin (pancreozymin), cholesterol, citric acid, citrulline, complement components, coagulation factors, coagulation proteins, complement components, c-peptide, c-reactive protein, creatine, creatinine, cyanide, 11-deoxycortisol, deoxyribonucleic acid, dihydrotestosterone, diphosphoglycerate (phosphate), or the like:

Further non-limiting examples of detectable blood components include to dopamine, enzymes, epidermal growth factor, epinephrine, ergothioneine, erythrocytes, erythropoietin, folic acid, fructose, furosemide glucuronide, galactoglycoprotein, galactose (children), gamma-globulin, gastric inhibitory peptide, gastrin, globulin, α-1-globulin, α-2-globulin, α-globulins, β-globulin, β-globulins, glucagon, glucosamine, glucose, immunoglobulins (antibodies), lipase p, lipids, lipoprotein (sr 12-20), lithium, low-molecular weight proteins, lysine, lysozyme (muramidase), α-2-macroglobulin, γ-mobility (non-immunoglobulin), pancreatic polypeptide, pantothenic acid, para-aminobenzoic acid, parathyroid hormone, pentose, phosphorated, phenol, phenylalanine, phosphatase, acid, prostatic, phospholipid, phosphorus, prealbumin, thyroxine-binding, proinsulin, prolactin (female), prolactin (male), proline, prostaglandins, prostate specific antigen, protein, protoporphyrin, pseudoglobulin I, pseudoglobulin II, purine, pyridoxine, pyrimidine nucleotide, pyruvic acid, CCL5 (RANTES), relaxin, retinol, retinol-binding protein, riboflavin, ribonucleic acid, secretin, serine, serotonin (5-hydroxytryptamine), silicon, sodium, solids, somatotropin (growth hormone), sphingomyelin, succinic acid, sugar, sulfates, inorganic, sulfur, taurine, testosterone (female), testosterone (male), triglycerides, triiodothyronine, tryptophan, tyrosine, urea, uric acid, water, miscellaneous trace components, or the like.

Non-limiting examples of α-Globulins include α1-acid glycoprotein, α1-antichymotrypsin, a 1-antitrypsin, α1B-glycoprotein, α1-fetoprotein, α1-microglobulin, α1T-glycoprotein, α2HS-glycoprotein, α2-macroglobulin, 3.1 S Leucine-rich α2-glycoprotein, 3.8 S histidine-rich α2-glycoprotein, 4 S α2,α1-glycoprotein, 8 S α3-glycoprotein, 9.5 S α1-glycoprotein (serum amyloid P protein), Corticosteroid-binding globulin, ceruloplasmin, GC globulin, haptoglobin (e.g., Type 1-1, Type 2-1, or Type 2-2), inter-α-trypsin inhibitor, pregnancy-associated α2-glycoprotein, serum cholinesterase, thyroxine-binding globulin, transcortin, vitamin D-binding protein, Zn-α2-glycoprotein, or the like. Non-limiting examples of β-Globulins include hemopexin, transferrin, β2-microglobulin, β2-glycoprotein I, β2-glycoprotein II, (C3 proactivator), β2-glycoprotein III, C-reactive protein, fibronectin, pregnancy-specific β1-glycoprotein, ovotransferrin, or the like. Non-limiting examples of immunoglobulins include immunoglobulin G (e.g., IgG, IgG₁, IgG₂, IgG₃, IgG₄), immunoglobulin A (e.g., IgA, IgA₁, IgA₂), immunoglobulin M, immunoglobulin D, immunoglobulin E, κ Bence Jones protein, γ Bence Jones protein, J Chain, or the like.

Non-limiting examples of apolipoproteins include apolipoprotein A-I (HDL), apolipoprotein A-II (HDL), apolipoprotein C-I (VLDL), apolipoprotein C-II, apolipoprotein C-III (VLDL), apolipoprotein E, or the like. Non-limiting examples of γ-mobility (non-immunoglobulin) include 0.6 S γ2-globulin, 2 S γ2-globulin, basic Protein B2, post-γ-globulin (γ-trace), or the like. Non-limiting examples of low-molecular weight proteins include lysozyme, basic protein B1, basic protein B2, 0.6 S γ2-globulin, 2 S γ2-globulin, post γ-globulin, or the like.

Non-limiting examples of complement components include C1 esterase inhibitor, C1q component, C1r component, C1s component, C2 component, C3 component, C3a component, C3b-inactivator, C4 binding protein, C4 component, C4a component, C4-binding protein, C5 component, C5a component, C6 component, C7 component, C8 component, C9 component, factor B, factor B (C3 proactivator), factor D, factor D (C3 proactivator convertase), factor H, factor H (β₁H), properdin, or the like. Non-limiting examples of coagulation proteins include antithrombin III, prothrombin, antihemophilic factor (factor VIII), plasminogen, fibrin-stabilizing factor (factor XIII), fibrinogen, thrombin, or the like.

Non-limiting examples of cell-Related Plasma Proteins include fibronectin, β-thromboglobulin, platelet factor-4, serum Basic Protease Inhibitor, or the like. Non-limiting examples of amyloid proteins (Non-Immunoglobulin) include amyloid-Related apoprotein (apoSAA1), AA (FMF) (ASF), AA (TH) (AS), serum amyloid P component (9.5 S 7α1-glycoprotein), or the like. Non-limiting examples of miscellaneous trace components include varcinoembryoriic antigen, angiotensinogen, or the like.

Further non-limiting examples of biomarkers in CSF include nucleic acid biomarkers, protein biomarkers, peptide biomarkers, or the like.

In an embodiment, the system 100 includes one or more sensor components 204 configured to detect an energy absorption, reflection, or transmission profile of a portion of a biological sample. For example, in an embodiment, the system 100 includes at least one sensor component 204 having a charge-coupled device for obtaining a spectral profile (e.g., a reflectance spectral profile, transmittance spectral profile, absorbance spectral profile, a spatial spectral profile, an image, or the like) of a biological sample. In an embodiment, the biomarker detection circuit 202 includes at least one sensor component 204. In an embodiment, the biomarker detection circuit 202 includes at least one sensor component 204 having a component identification code and configured to implement instructions addressed to the sensor component 204 according to the component identification code.

In an embodiment, the system 100 includes one or more sensor components 204 that obtain spectral information from one or more biomarkers within a sample, while varying at least one of a frequency, intensity, polarization, wavelength, or spectral power density associated with an interrogation energy source (e.g., electromagnetic energy source, optical energy source, acoustic energy source, electrical energy source, thermal energy source, or the like). For example, in an embodiment, the sensor component 204 detects scattered energy associated with biomarkers within CSF interrogated by an electromagnetic energy stimulus. In an embodiment, the system 100 includes one or more sensor components 204 configured to detect one or more optical properties of a tissue or biological fluid.

In an embodiment, the sensor component 204 includes one or more sensors 206. For example, in an embodiment, the biomarker detection circuit 202 includes one or more sensors 206 configured to actively detect, diagnose, or treat a neuropsychiatric disorders (e.g., anxiety disorder, bipolar disorder, depression, effective disorder, mood spectrum disorder, schizophrenia, or the like), a neurodegenerative disorder (e.g., Alzheimer's Disease, Huntington's disease, Parkinson's disease, other dementias, or the like), or the like. In an embodiment, the biomarker detection circuit 202 includes one or more sensors 206 operably coupled to an interior of the one or more fluid-flow passageways 108.

Non-limiting examples of sensors 206 include biosensors, detectors, refractive index detectors, blood volume pulse sensors, conductance sensors, electrochemical sensors, fluorescence sensors, force sensors, heat sensors (e.g., thermistors, thermocouples, or the like), high resolution temperature sensors, differential calorimeter sensors, optical sensors, goniometry sensors, potentiometer sensors, resistance sensors, respiration sensors, sound sensors (e.g., ultrasound), Surface Plasmon Band Gap sensor (SPRBG), physiological sensors, surface plasmon sensors, or the like. Further non-limiting examples of sensors 206 include affinity sensors, bioprobes, biostatistics sensors, enzymatic sensors, in-situ sensors (e.g., in-situ chemical sensor), ion sensors, light sensors (e.g., visible, infrared, or the like), microbiological sensors, microhotplate sensors, micron-scale moisture sensors, nanosensors, optical chemical sensors, single particle sensors, or the like. Further non-limiting examples of sensors include chemical sensors, cavitand-based supramolecular sensors, deoxyribonucleic acid sensors (e.g., electrochemical DNA sensors, or the like), supramolecular sensors, or the like.

In an embodiment, the biomarker detection circuit 202 includes one or more sensors 206 that detect changes in a spectral profile of one or more CSF measurands obtained at a plurality of sequential time points. Changes in CSF composition can depend on, for example, blood proteome, CSF circulation alterations, or metabolic processes, as well as the brain's physiological or pathological status. In an embodiment, the system 100 includes one or more sensors 206 configured to detect, describe, or track target markers associated with one or more acute, chronic, or progressive conditions, as well as to perform CSF proteomic, peptidomic, metabolic, or biomarker analysis.

In an embodiment, the biomarker detection circuit 202 includes a computing device 208 configured to process sensor measurand information and configured to cause the storing of the measurand information in a data storage medium. In an embodiment, the biomarker detection circuit 202 includes one computing device 208 operably coupled to one or more sensors 206 and is configured to determine a sampling regimen.

Further non-limiting examples of sensors 206 include electrochemical detectors, fluorescent detectors, light scattering detectors, mass spectroscopy detectors nuclear magnetic resonance detectors, near-infra red detectors, radiochemical detectors, refractive index detectors, ultra-violet detectors, or the like. Further non-limiting examples of sensors 206 include chemical transducers, ion sensitive field effect transistors (ISFETs), ISFET pH sensors, membrane-ISFET devices (MEMFET), microelectronic ion-sensitive devices, potentiometric ion sensors, quadruple-function ChemFET (chemical-sensitive field-effect transistor) integrated-circuit sensors, sensors with ion-sensitivity and selectivity to different ionic species, or the like. Further non-limiting examples of the one or more sensors 206 can be found in the following documents: U.S. Pat. No. 7,396,676 (issued Jul. 8, 2008) and U.S. Pat. No. 6,831,748 (issued Dec. 14, 2004); each of which is incorporated herein by reference.

In an embodiment, the sensor component 206 is configured to determine at least one spectral parameter associated with one or more imaging probes (e.g., chromophores, fluorescent agents, fluorescent marker, fluorophores, molecular imaging probes, quantum dots, or radio-frequency identification transponders (RFIDs), x-ray contrast agents, or the like). In an embodiment, the sensor component 206 is configured to determine at least one characteristic associated with one or more imaging probes attached, targeted to, conjugated, bound, or associated with at least one neuropsychiatric disorder biomarker. In an embodiment, the one or more imaging probes include at least one carbocyanine dye label. In an embodiment, the sensor component 206 is configured to determine at least one characteristic associated with one or more imaging probes attached, targeted to, conjugated, bound, or associated with at least one biomarker or biological sample component.

In an embodiment, the one or more sensors 206 include one or more acoustic transducers, electrochemical transducers, optical transducers, piezoelectrical transducers, or thermal transducers. In an embodiment, the one or more sensors 206 include one or more thermal detectors, photovoltaic detectors, or photomultiplier detectors. In an embodiment, the one or more sensors 206 include one or more charge-coupled devices, complementary metal-oxide-semiconductor devices, photodiode image sensor devices, whispering gallery mode (WGM) micro cavity devices, or scintillation detector devices. In an embodiment, the one or more sensors 206 include one or more ultrasonic transducers.

In an embodiment, the one or more sensors 206 include at least one surface plasmon resonance (SPR) sensor. Non-limiting examples of SPR sensors include surface localized surface plasmon resonance sensors (LSPR); tunable SPR or LSPR sensors (e.g., SPR or LSPR sensors including dynamic tunable metal-dielectric materials, thermally tunable SPR or LSPR sensors; tunable fiber-optic SPR or LSPR sensors including indium tin oxide coatings; tunable SPR or LSPR sensors including elastomeric substrates; wavelength-tunable SPR or LSPR sensors; or the like); surface-plasmon-polariton-based sensors; optical SPR or LSPR sensors, or the like.

In an embodiment, the biomarker detection circuit 202 includes one or more SPR or LSPR sensors that are actively-tuned by controllably adjusting the refractive index of a material forming part of a plasmon supporting surface region. In an embodiment, the SPR or LSPR sensors are tuned by adjusting the dielectric constant of a material forming part of a plasmon supporting surface region. In an embodiment, the SPR or LSPR sensors include an elastomeric substrate configured to affect a resonance condition of surface plasmon polaritons in the presence of a mechanical strain or in the presence of an applied potential. In an embodiment, the one or more sensors 206 include an LSPR sensing array including solid detectors that provide real-time-parallel-detection of multiple components of a biological sample. In an embodiment, the one or more sensors 206 include a light transmissive support and a reflective metal layer. In an embodiment, the biomarker detection circuit 202 includes at least one sensor component 204 having one or more sensors 206 and at least one computing device 208 operably coupled to the at least one sensor component 204.

In an embodiment, the one or more sensors 206 include one or more density sensors, optical density sensors, refractive index sensors. In an embodiment, one or more sensors 206 include at least one fiber optic refractive index sensor. In an embodiment, one or more sensors 206 include at least one a surface plasmon interferometer. In an embodiment, the surface plasmon interferometer is configured to detect changes in a refractive index based on the interference of two surface-plasmon. In an embodiment, the one or more sensors 206 include one or more acoustic biosensors, amperometric biosensors, calorimetric biosensors, optical biosensors, or potentiometric biosensors. In an embodiment, the one or more sensors 206 include one or more fluid flow sensors, differential electrodes, biomass sensors, immuno sensors, functionalized cantilevers, or the like.

In an embodiment, the biomarker detection circuit 202 includes at least one SPR microarray sensor. In an embodiment, the at least one SPR microarray sensor includes an array of micro-regions modified to capture target molecules.

In an embodiment, the biomarker detection circuit 202 includes data storage circuitry configured to store biomarker profile time series information. In an embodiment, the biomarker detection circuit 202 includes data storage circuitry configured to store paired and unpaired biomarker profile data. In an embodiment, the biomarker detection circuit 202 includes data storage circuitry configured to store biomarker profile time series information.

In an embodiment, the biomarker detection circuit 202 includes circuitry configured to detect at least one of an energy absorption, energy reflection, or energy transmission spectra. For example, in an embodiment, the biomarker detection circuit 202 includes a spectrometer 210. In an embodiment, the spectrometer 210 includes a single or multi-wavelength interrogation mode component and a detector array. In an embodiment, the biomarker detection circuit 202 includes circuitry having one or more sensors 206 that detect at least one of absorption coefficient information, extinction coefficient information, or scattering coefficient information associated with the CSF received within the one or more fluid-flow passageways 108.

In an embodiment, the biomarker detection circuit 202 includes circuitry having one or more components operably coupled (e.g., communicatively coupled, electromagnetically, magnetically, ultrasonically, optically, inductively, electrically, capacitively coupleable, or the like) to each other. In an embodiment, circuitry includes one or more remotely located components. In an embodiment, remotely located components can be operably coupled via wireless communication. In an embodiment, remotely located components can be operably coupled via one or more receivers 203, transmitters 205, transceivers 207, or the like. In an embodiment, circuitry includes, among other things, one or more computing devices 208 such as a processor (e.g., a microprocessor) 212, a central processing unit (CPU) 214, a digital signal processor (DSP) 216, an application-specific integrated circuit (ASIC) 218, a field programmable gate array (FPGA) 220, or the like, or any combinations thereof, and can include discrete digital or analog circuit elements or electronics, or combinations thereof. In an embodiment, circuitry includes one or more field programmable gate arrays 220 having a plurality of programmable logic components. In an embodiment, circuitry includes one or more application specific integrated circuits having a plurality of predefined logic components.

In an embodiment, circuitry includes one or more memories 222 that, for example, store instructions or data, for example, volatile memory (e.g., Random Access Memory (RAM) 224, Dynamic Random Access Memory (DRAM), or the like), non-volatile memory (e.g., Read-Only Memory (ROM) 226, Electrically Erasable Programmable Read-Only Memory (EEPROM), Compact Disc Read-Only Memory (CD-ROM), or the like), persistent memory, or the like. Further non-limiting examples of one or more memories 222 include Erasable Programmable Read-Only Memory (EPROM), flash memory, or the like. The one or more memories 222 can be coupled to, for example, one or more computing devices 208 by one or more instruction, data, or power buses.

In an embodiment, circuitry includes one or more computer-readable media drives 215, interface sockets, Universal Serial Bus (USB) ports, memory card slots, or the like, and one or more input/output components 213 such as, for example, a graphical user interface, a display 217, a keyboard 221, a keypad, a trackball, a joystick, a touch-screen, a mouse, a switch, a dial, or the like, and any other peripheral device. In an embodiment, circuitry includes one or more user input/output components 213 that operably coupled to at least one computing device 208 to control (electrical, electromechanical, software-implemented, firmware-implemented, or other control, or combinations thereof) at least one parameter associated with acquiring at least one biomarker profile of CSF proximate (e.g., received within, near, etc.) an implantable device 102. In an embodiment, the system 100 includes, among other things, one or more modules optionally operable for communication with one or more input/output components 213 that are configured to relay user output and/or input. In an embodiment, a module includes one or more instances of electrical, electromechanical, software-implemented, firmware-implemented, or other control devices. Such device include one or more instances of memory 222; computing devices 208; antennas; power or other supplies; logic modules or other signaling modules; gauges or other such active or passive detection components; piezoelectric transducers, shape memory elements, micro-electro-mechanical system (MEMS) elements, or other actuators.

The computer-readable media drive 211 or memory slot can be configured to accept signal-bearing medium (e.g., computer-readable memory media, computer-readable recording media, or the like). In an embodiment, a program for causing the system 100 to execute any of the disclosed methods can be stored on, for example, a computer-readable recording medium (CRMM) 215, a signal-bearing medium, or the like. Non-limiting examples of signal-bearing media include a recordable type medium such as a magnetic tape, floppy disk, a hard disk drive, a Compact Disc (CD), a Digital Video Disk (DVD), Blu-Ray Disc, a digital tape, a computer memory, or the like, as well as transmission type medium such as a digital and/or an analog communication medium (e.g., a fiber optic cable, a waveguide, a wired communications link, a wireless communication link (e.g., transmitter, receiver, transceiver, transmission logic, reception logic, etc.), etc.). Further non-limiting examples of signal-bearing media include, but are not limited to, DVD-ROM, DVD-RAM, DVD+RW, DVD-RW, DVD-R, DVD+R, CD-ROM, Super Audio CD, CD-R, CD+R, CD+RW, CD-RW, Video Compact Discs, Super Video Discs, flash memory, magnetic tape, magneto-optic disk, MINIDISC, non-volatile memory card, EEPROM, optical disk, optical storage, RAM, ROM, system memory, web server, or the like.

In an embodiment, the biomarker detection circuit 202 includes circuitry having one or more databases 228. In an embodiment, a database 228 includes mental disorder state marker information, mental disorder trait information, or heuristically determined mental disorder information. In an embodiment, a database 228 includes reference biomarker information (e.g., reference biomarker spectral response information, reference biomarker optical response information, or the like), reference neuropsychiatric disorder spectral information, or reference neurodegenerative disorder spectral information. In an embodiment, a database 228 includes at least one of diseased state indication information, diseased tissue indication information, infection indication information, or inflammation indication information.

In an embodiment, a database 228 includes one or more heuristically determined parameters associated with at least one in vivo or in vitro determined metric. In an embodiment, a database 228 includes at least one of predisposition for a mental disorder indication information, mental disorder state indication information, or mental disorder trait indication information. In an embodiment, a database 228 includes at least one of biomarker absorption coefficient data, biomarker extinction coefficient data, or biomarker scattering coefficient data. In an embodiment, a database 228 includes stored reference data such as reference infectious agent marker data, reference mental disorder marker data, reference CSF component data, reference blood component data, or the like.

In an embodiment, a database 228 includes information associated with a disease state of a biological subject. In an embodiment, a database 228 includes measurement data. In an embodiment, a database 228 includes at least one of cryptographic protocol information, regulatory compliance protocol information (e.g., FDA regulatory compliance protocol, information, or the like), regulatory use protocol information, authentication protocol information, authorization protocol information, delivery regimen protocol information, activation protocol information, encryption protocol information, decryption protocol information, treatment protocol information, or the like. In an embodiment, a database 228 includes at least one of interrogation energy control delivery information, energy emitter control information, power control information, or the like. In an embodiment, a database 228 includes reference data associated with a formation or presence of a pathological condition indicative of at least one of Alzheimer's disease, amyotrophic lateral sclerosis, bipolar disorder, Creutzfeldt-Jakob disease, dementia, depression, encephalitis, HIV associated dementia, ischemia, major depressive disorder, meningitis, multiple sclerosis, neuropsychiatric disorder, Parkinson's disease, psychosis, schizophrenia, sclerosis, suicidal tendency, or traumatic brain injury.

In an embodiment, the biomarker detection circuit 202 includes circuitry having one or more data structures (e.g., physical data structures) 230. In an embodiment, a data structure 230 includes at least one of mental disorder state marker information, mental disorder trait information, or heuristically determined mental disorder information stored thereon. In an embodiment, a data structure 230 includes at least one of reference biomarker information (e.g., reference biomarker spectral response information, reference biomarker optical response information, or the like), reference neuropsychiatric disorder spectral information, or reference neurodegenerative disorder spectral information stored thereon. In an embodiment, a data structure 230 includes at least one of psychosis state marker information, psychosis trait marker information, or psychosis indication information.

In an embodiment, the biomarker detection circuit 202 includes circuitry configured to detect an optical energy absorption, reflection, or transmission profile associated with CSF received within the one or more fluid-flow passageways 108. For example, in an embodiment, the biomarker detection circuit 202 includes a spectrometer 210 that measures absorption, reflection, or transmission of radiation, as a function of frequency, wavelength, or the like, of a biological sample. In an embodiment, the biomarker detection circuit 202 includes circuitry configured to detect at least one of protein biomarker spectral information or peptide biomarker spectral information of CSF received within the one or more fluid-flow passageways 108. For example, in an embodiment, the biomarker detection circuit 202 includes circuitry that detects at least one of a protein biomarker profile or a peptide biomarker profile of CSF received within the one or more fluid-flow passageways 108 by monitoring changes to a resonance condition of a plasmon-resonance-supporting portion of a sensor 206.

In an embodiment, the biomarker detection circuit 202 includes circuitry configured to detect electromagnetic absorption coefficient information. For example, in an embodiment, the biomarker detection circuit 202 includes circuitry having a charge-coupled device that detects fluorescence information. In an embodiment, the biomarker detection circuit 202 includes circuitry configured to detect an optical energy absorption profile associated with CSF received within the one or more fluid-flow passageways 108. In an embodiment, the biomarker detection circuit 202 includes at least one component configured to generate a biomarker profile image of CSF applied to an array. In an embodiment, the biomarker detection circuit 202 includes at least one component configured to generate an n-dimensional expression profile vector of a portion of CSF received within the one or more fluid-flow passageways 108.

In an embodiment, the biomarker detection circuit 202 includes at least one of a biomedical array or a chemical compound array. In an embodiment, the biomarker detection circuit 202 includes at least one microarray. In an embodiment, the biomarker detection circuit 202 includes at least one of an antibody array, a deoxyribonucleic acid array, a ribonucleic acid array, a peptide array, or a protein array. In an embodiment, the biomarker detection circuit 202 includes at least one protein in situ array.

In an embodiment, the biomarker detection circuit 202 includes at least one sensor component 204 operably coupled to a biomedical array or a chemical compound array. For example, in an embodiment, the biomarker detection circuit 202 includes at least one sensor component 204 operably coupled to at least one psychotic disorder biomarker array or neuropsychiatric disorder peptide biomarker array. In an embodiment, the biomarker detection circuit 202 includes at least one transceiver 207 operably coupled to a microarray. In an embodiment, the transceiver 207 transmits electromagnetic energy to the microarray and receives electromagnetic energy from the microarray.

In an embodiment, the biomarker detection circuit 202 includes an array of selectively actuatable sensors. For example, in an embodiment, the biomarker detection circuit 202 includes at least one computing device 208 operably coupled to an array of sensors 206. During operation, the computing device 208 is configured to activate one or more sensors 206 in sensor array in response to at least one of psychosis state information, psychosis trait information, psychosis prodromal information, or psychosis indication information. In an embodiment, the biomarker detection circuit 202 includes an array of sensors including one or more activation components that selectively expose one or more sensors 206 of the array of sensors to CSF received within the one or more fluid-flow passageways 108. In an embodiment, the biomarker detection circuit 202 includes circuitry that selectively exposes one or more sensors 206 of an array of sensor to CSF received within the one or more fluid-flow passageways 108.

Referring to FIG. 2, in an embodiment, the system 100 includes, among other things, at least one implantable device 102 including a biomarker identification circuit 232 (e.g., a mental disorder biomarker identification circuit, etc.). In an embodiment, the biomarker identification circuit 232 is configured to compare a detected biomarker profile of the CSF to filtering information 234 and to generate a response based on the comparison. For example, in an embodiment, the biomarker identification circuit 232 compares an input associated with a detected mental disorder biomarker profile to a database 228 of stored reference values, and generates a response based in part on the comparison. In an embodiment, the biomarker identification circuit 232 is configured to compare sensor output information to a database 228 of stored reference values, and to generate a response based in part on the comparison. In an embodiment, the biomarker identification circuit 232 is configured to acquire in vivo CSF spectral information of CSF proximate a surface of an indwelling shunt. In an embodiment, the biomarker identification circuit 232 is configured to compare a detected biomarker profile of the CSF to user-specific filtering information 235 (e.g., user-specific mental disorder biomarker information, user-specific reference biomarker information, user-specific reference biomarker threshold level information, or the like) information and to generate a response based on the comparison.

In an embodiment, the biomarker identification circuit 232 includes a computing device 208 operable to compare an output of one or more of a plurality of logic components and to determine at least one parameter associated with a cluster centroid deviation derived from the comparison. In an embodiment, the biomarker identification circuit 232 is configured to compare a measurand associated with a biological sample to a threshold value associated with a mental disorder spectral model and to generate a response based on the comparison. In an embodiment, the biomarker identification circuit 232 is configured to generate a response based on the comparison of a measurand that modulates with a detected heartbeat of the biological subject to a target value associated with a spectral model. In an embodiment, during operation, the biomarker identification circuit 232 compares a measurand associated with one or more mental disorder biomarkers to threshold values associated with a spectral model and to generate a real-time estimation of a disease state based on the comparison. In an embodiment, the biomarker identification circuit 232 is configured to compare an input associated with at least one characteristic associated with, for example, a biological sample proximate the implantable device 102 to a database 228 of stored reference values, and to generate a response based in part on the comparison.

In an embodiment, the biomarker identification circuit 232 includes, among other things, one or more computing devices 208 for accessing discrete data structures 230 having filtering information 234 stored thereon. For example, in an embodiment, the biomarker identification circuit 232 includes one or more computing devices 208 that access discrete data structures 230 having information associated with a disease state of a biological subject. In an embodiment, one or more of the data structures 230 include measurement data associated with at least one of no-psychosis state, a pre-psychosis state, or a psychosis state. In an embodiment, one or more of the data structures 230 include measurement data associated with a prodromal state of a psychotic disorder. In an embodiment, the biomarker identification circuit 232 includes one or more data structures 230 having at least one of user-specific information or user related information stored thereon.

In an embodiment, the biomarker identification circuit 232 includes one or more computer-readable memory media 215 having filtering information 234 configured as a data structure.

In an embodiment, the filtering information 234 includes characteristic spectral information of CSF biomarkers or characteristic pathology information indicative of at least one of Alzheimer's disease, amyotrophic lateral sclerosis, bipolar disorder, Creutzfeldt-Jakob disease, dementia, depression, encephalitis, HIV associated dementia, ischemia, major depressive disorder, meningitis, multiple sclerosis, neuropsychiatric disorder, Parkinson's disease, psychosis, schizophrenia, sclerosis, suicidal tendency, traumatic brain injury, or the like. For example, in an embodiment, the filtering information 234 includes at least one of neuroendocrine VGF-derived peptides spectral information or transthyretin proteins spectral information. In an embodiment, the filtering information 234 includes at least one of upregulation model information associated with a VGF23-62 peptide, decreased expression model information associated with a VGF26-62 peptide, user-specific upregulation information associated with a VGF23-62 peptide, or user-specific decreased expression information associated with a VGF26-62 peptide.

In an embodiment, the biomarker identification circuit 232 includes a receiver 203 configured to acquire filtering information 234. In an embodiment, the receiver 203 is configured to request filtering information 234. In an embodiment, the receiver 203 is configured to receive a request to transmit at least one of filtering information 234, detected protein biomarker profile information, detected peptide biomarker profile information, or comparison information. In an embodiment, the biomarker identification circuit 232 includes a transmitter 205 configured to send comparison information associated with a comparison of a detected biomarker profile of CSF received within the implantable device 102 to filtering information 234.

In an embodiment, the biomarker identification circuit 232 includes a transceiver 207 configured to transmit information relating to protein biomarker profile detection or peptide biomarker profile detection. For example, in an embodiment, the biomarker identification circuit 232 includes a transceiver 207 that transmits detected protein biomarker profile information or detected peptide biomarker profile information and receives instructions in response to the transmitted detected protein biomarker profile information or the transmitted detected peptide biomarker profile information. In an embodiment, the biomarker identification circuit 232 includes a transceiver 207 to receive instructions in response to transmitted detected protein biomarker profile information or the transmitted detected peptide biomarker profile information. In an embodiment, the biomarker identification circuit 232 includes a transceiver 207 configured to transmit information relating to protein biomarker profile detection or peptide biomarker profile detection

In an embodiment, the biomarker identification circuit 232 includes at least one computing device 208 that controls the transceiver 207. In an embodiment, during operation, the transceiver 207 reports information generated by the biomarker identification circuit 232 when a detected biomarker profile of CSF received within the implantable device 102 satisfies a threshold criterion, for example, when a detected biomarker profile meets or exceeds a target range.

In an embodiment, the transceiver 207 is configured to report information generated by the biomarker identification circuit 232 based on a time for which a threshold criterion is met. For example, in an embodiment, the transceiver 207 reports status information at target time intervals. In an embodiment, the transceiver 207 is configured to report status information at a plurality of time intervals and to enter a receive mode for a period after transmitting the report information. In an embodiment, the transceiver 207 is configured to operate in a low-power mode when not reporting information or receiving instructions. In an embodiment, the transceiver 207 is configured to report status information at regular or irregular time intervals. In an embodiment, the transceiver 207 is configured to report status information when relationship between measurands detected at different times exceeds threshold. In an embodiment, the transceiver 207 is configured to report status information when a relationship between two or more different biomarkers exceeds a threshold criterion. In an embodiment, the transceiver 207 is configured to report status information when a difference between a measurand and a user-related target value exceeds a threshold criterion.

In an embodiment, the biomarker identification circuit 232 includes one or more computing devices 208 operable to compare a change associated with a biomarker compositing, biomarker level, or the like of CSF received within the implantable device 102 to the filtering information 234. In an embodiment, at least one computing devices 208 is operable to compare a change associated with one or more biomarker levels of CSF received within the implantable device 102 to the filtering information 234. In an embodiment, at least one computing devices 208 is operable to compare a change in a concentration of one or more biomarkers of CSF received within the implantable device 102 to the filtering information 234. In an embodiment, at least one computing devices 208 is operable to compare a relative rate of change of one or more biomarkers of CSF within the implantable device 102 to the filtering information 234. In an embodiment, the biomarker identification circuit 232 includes one or more computing devices 208 operable to compare a relationship between two or more biomarkers of the CSF received within the one or more fluid-flow passageways 108 to the filtering information 234 (e.g., mental disorder filtering information, or the like).

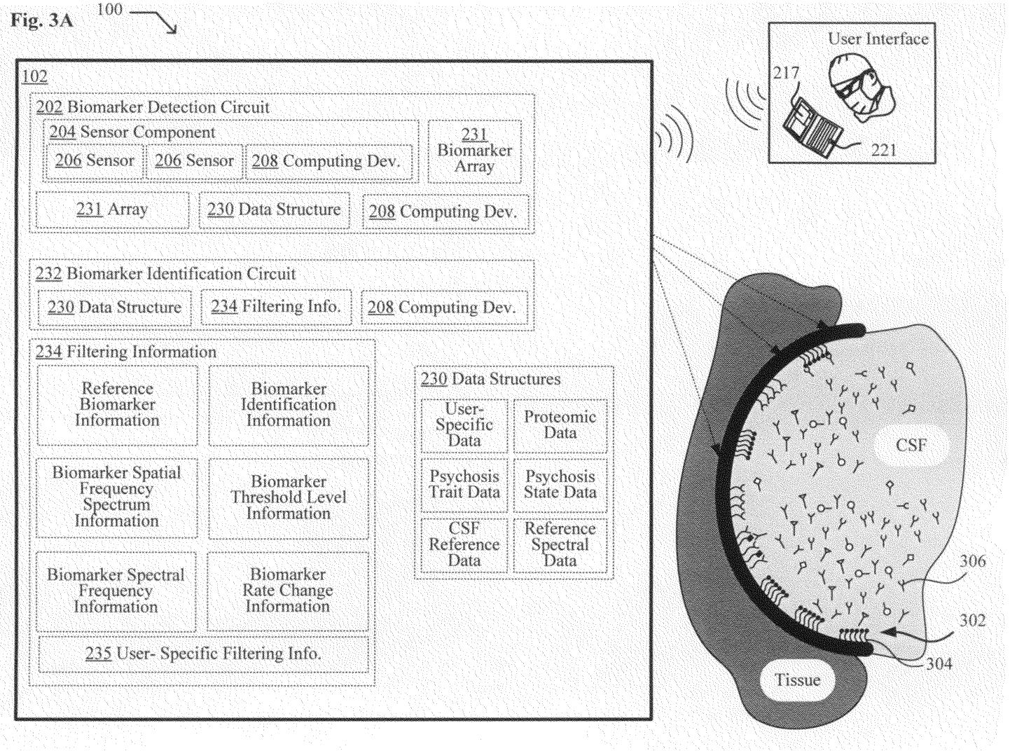

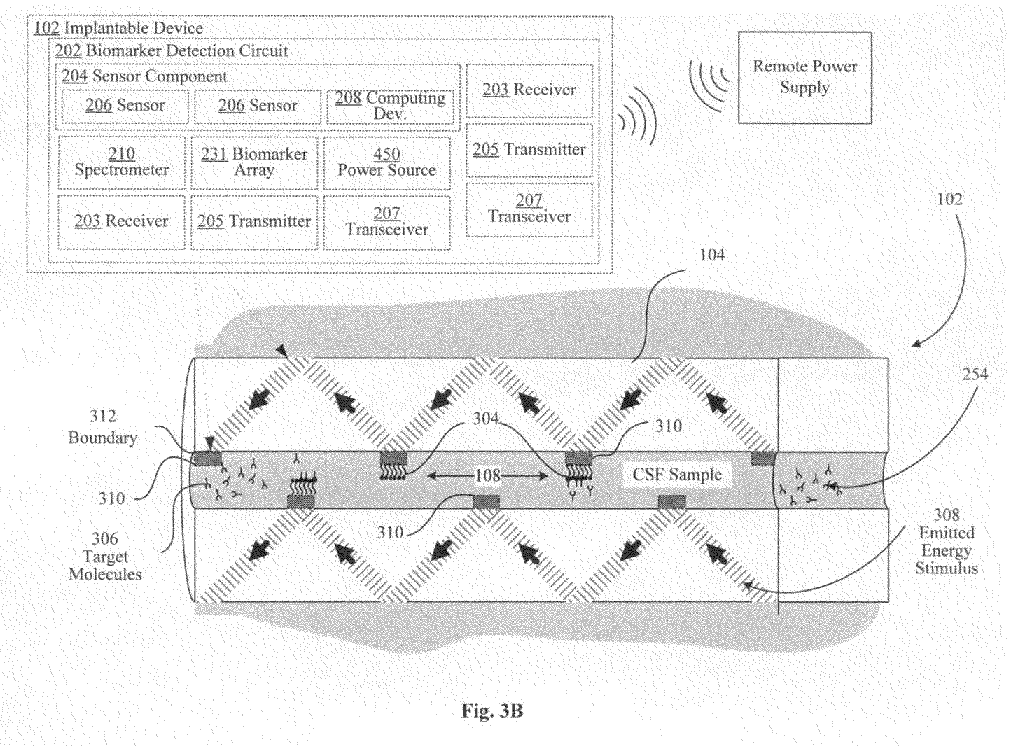

Referring to FIG. 3A, in an embodiment, the system 100 is configured to detect one or more target markers present in a sample (e.g., tissue, biological fluid, infections agent, biomarker, or the like). For example, in an embodiment, the system 100 includes an implantable device 102 having one or more sensor components 204 for detecting one or more target markers present in a sample. In an embodiment, the implantable device 102 includes one or more sensor components 204 for detecting one or more biomarkers associated with a mental disorder (e.g., neuropsychiatric disorders, neurodegenerative disorders, or the like). For example, in an embodiment, the implantable device 102 includes at least one sensor component 204 having a biological molecule capture layer 302 incorporating one or more targeting moieties 304 that selectively target a marker 306 associated with a mental disorder. In an embodiment, the sensors component 204 is configured to detect, in real-time, one or more target markers 306 present in, for example, CSF. Cerebrospinal fluid frequents the ventricles of the brain and the subarachnoid spaces and closely contacts the brain's extracellular fluid. It circulates within the central nervous system playing an essential physiological role in, for example, homeostasis of neuronal cells.

Cerebrospinal fluid includes a protein diversity that results, among other things, from both filtration of serum through the blood-brain barrier and production or secretion of neuronal peptides and proteins. In an embodiment, the implantable device 102 includes at least one sensor component 204 having a biological molecule capture layer 302 incorporating one or more targeting moieties 304 that selectively target CSF biomarkers associated with Alzheimer's disease, amyotrophic lateral sclerosis, bipolar disorder, Creutzfeldt-Jakob disease, dementia, depression, encephalitis, HIV associated dementia, ischemia, major depressive disorder, meningitis, multiple sclerosis, neuropsychiatric disorder, Parkinson's disease, psychosis, schizophrenia, sclerosis, suicidal tendency, traumatic brain injury, or the like.

In an embodiment, the implantable device 102 monitors one or more components in CSF or changes in its composition to determine information about the brain's metabolism and a person's disease state. In an embodiment, the implantable device 102 includes one or more sensor components 204 that monitor one or more target markers 306 associated with dementia. For example, in an embodiment, at least one of the one or more sensor components 204 includes a biomarker capture layer 302 having a monoclonal antibody (e.g., clone DC11, product number A8855, Sigma-Aldrich, S. Louis, Mo.) that specifically binds to a form of tau selectively observed in Alzheimer's disease.

In an embodiment, the implantable device 102 includes one or more sensor components 204 having biological molecule capture layer 302 including an array of different binding molecules that specifically bind to one or more target molecules. For example, in an embodiment, the biomarker detection circuit 202 includes at least one SPR microarray sensor having an array of micro-regions configured to capture target molecules. In an embodiment, the one or more sensor components 204 include a biological molecule capture layer 302 incorporating a plurality of targeting moieties 304 for identifying one or more factors associated with a specific disease state, pathology, or condition.

Non-limiting examples of targeting moieties 304 include antibodies or fragments thereof, oligonucleotide or peptide based aptamers, receptors or parts thereof, receptor ligands or parts thereof, lectins, artificial binding substrates formed by molecular imprinting, biomolecules, humanized targeting moieties, mutant or genetically engineered proteins, mutant or genetically engineered protein binding domains, adhesion proteins, integrins, mucins, fibronectins, or substrates (e.g., poly-lysine, collagen, Matrigel, fibrin) that interact with components of tissues or cells, or the like. In an embodiment, the one or more sensor components 204 include a biological molecule capture layer 302 incorporating one or more antibodies or fragments thereof for identifying one or more factors associated with a specific disease state, pathology, or condition.

Further non-limiting examples of targeting moieties 304 include monoclonal antibodies, polyclonal antibodies, chimeric antibodies, rabbit antibodies, chicken antibodies, mouse antibodies, human antibodies, humanized antibodies or antibody fragments, Fab fragments of antibodies, F(ab′)2 fragments of antibodies, single-chain variable fragments (scFvs) of antibodies, diabody fragments (dimers of scFv fragments), minibody fragments (dimers of scFvs-CH3 with linker amino acid), or the like. Further examples of antibodies or fragments include bispecific antibodies, trispecific antibodies, single domain antibodies (e.g., camel and llama VHH domain), lamprey variable lymphocyte receptor proteins, antibodies based on proteins or protein motifs (for example lipocalins, fibronectins, ankyrins and src-homology domains.

In an embodiment, the targeting moieties 304 include one or more antibodies. Non-limiting examples of antibodies include immunoglobulin molecules including four polypeptide chains, two heavy (H) chains, and two light (L) chains inter-connected by disulfide bonds. In an embodiment, each heavy chain includes a heavy chain variable region (VH) and a heavy chain constant region. In an embodiment, the heavy chain constant region includes three domains, CH1, CH2 and CH3. In an embodiment, each light chain includes a light chain variable region (VL) and a light chain constant region. The light chain constant region includes one domain, CL. The VH and VL regions can be further subdivided into regions of hypervariability, termed complementarity determining regions (CDRs), interspersed with regions that are more conserved, termed framework regions (FR). In an embodiment, each VH and VL includes three complementarity determining regions and four framework regions, arranged from amino-terminus to carboxy-terminus in the following order: FR1, CDR1, FR2, CDR2, FR3, CDR3, FR4. (See, e.g., U.S. Pat. No. 7,504,485 (issued Mar. 17, 2009); which is incorporated herein by reference). In an embodiment, pairing of VH and VL together forms a single antigen-binding portion of the antibody.

In an embodiment, the targeting moieties 304 include one or more antibody fragments. Non-limiting examples of antibody fragments include fragments of an antibody that retain the ability to specifically bind to an antigen (e.g., antigen-binding portions). It has been shown that the antigen-binding function of an antibody can be performed by fragments of a full-length antibody. Non-limiting examples of binding fragments include single domain antibodies (dAb) fragments (e.g., those including a single VH domain), F(ab′)2 fragments (e.g., a bivalent fragment including two Fab fragments linked by a disulfide bridge at the hinge region), Fab fragments (e.g., a monovalent fragment including VL, VH, CL and CH1 domains), Fd fragments (e.g., those including VH and CH1 domains), Fv fragments (e.g., those including VL and VH domains of a single arm of an antibody), single chain Fv (linear fragment containing VH and VL regions separated by a short linker), diabodies (two single chain Fv fragments separated by short linkers), or the like. (See e.g., the following documents: Bird et al., Science 242:423-426 (1988); Ward et al., Nature 341:544-546 (1989); and Huston et al., Proc. Natl. Acad. Sci. USA 85:5879-5883 (1988); each of which is incorporated herein by reference). In an embodiment, the targeting moieties 304 include single chain or multiple chain antigen-recognition motifs, epitopes, or mimotopes. In an embodiment, the multiple chain antigen-recognition motifs, epitopes, or mimotopes can be fused or unfused.

Non-limiting examples of diabodies include bivalent, bispecific antibodies having VH and VL domains expressed on a single polypeptide chain, but using a linker that is too short to allow for pairing between the two domains on the same chain (thereby forcing the domains to pair with complementary domains of another chain and creating two antigen binding sites). See e.g., the following documents: Holliger, P., et al., Proc. Natl. Acad. Sci. USA 90:6444-6448 (1993); Poljak, R. J., et al., Structure 2:1121-1123 (1994); each of which is incorporated herein by reference.

In an embodiment, an antibody or antigen-binding portion thereof can be part of a larger immunoadhesion molecule, formed by covalent or non-covalent association of the antibody or antibody portion with one or more other proteins or peptides. (See e.g., the following documents: Kipriyanov, S. M., et al., Human Antibodies and Hybridomas 6:93-101 (1995) and Kipriyanov, S. M., et al., Mol. Immunol. 31:1047-1058 (1994); each of which is incorporated herein by reference). Antibody portions, such as Fab and F(ab′)2 fragments, are prepared from whole antibodies using conventional techniques, such as papain or pepsin digestion, respectively, of whole antibodies. Antibodies, antibody portions and immunoadhesion molecules, or the like can be obtained using standard recombinant DNA techniques.

Further non-limiting examples of antibodies or fragments thereof include antibodies or fragments thereof generated using, for example, standard methods such as those described by Harlow & Lane (Antibodies: A Laboratory Manual, Cold Spring Harbor Laboratory Press; 1st edition (1988); which is incorporated herein by reference). In an embodiment, an antibody or fragment thereof can be generated using phage display technology. (See, e.g., Kupper, et al. BMC Biotechnology 5:4 (2005); which is incorporated herein by reference). An antibody or fragment thereof could also be prepared using, for example, in silico design. See, e.g., Knappik et al., J. Mol. Biol. 296: 57-86 (2000); which is incorporated herein by reference.

In an embodiment, the targeting moieties 304 include at least one NANOBODY (e.g., single domain antibodies, single-chain antibody fragments (VHH), NANOBODIES (Ablynx nv Belgium), or the like, or fragments thereof; see, e.g., Harmsen, et al., Appl. Microbiol. Biotechnol. 77:31-22 (2007); which is incorporated herein by reference. In an embodiment, the targeting moieties 304 include at least one heavy chain, single N-terminal domain antibody that does not require domain pairing for antigen recognition.

In an embodiment, the implantable device 102 includes one or more sensor components 204 having a biological molecule capture layer 302 incorporating one or more oligonucleotide RNA or DNA based aptamers for identifying one or more factors associated with a specific disease state, pathology, or condition. Aptamers include oligonucleotides (DNA or RNA) or peptide molecules that can bind to a wide variety of entities (e.g., metal ions, small organic molecules, proteins, or cells) with high selectivity, specificity, and affinity. Aptamers can be isolated from a large library of about 10¹⁴ to about 10¹⁵ random oligonucleotide sequences using an iterative in vitro selection procedure often termed “systematic evolution of ligands by exponential enrichment” (SELEX). (See, e.g., Cao, et al., Current Proteomics 2:31-40, 2005; Proske, et al., Appl. Microbiol. Biotechnol. 69:367-374 (2005); Jayasena Clin. Chem. 45:1628-1650 (1999); each of which is incorporated herein by reference). In an embodiment, aptamers include those synthetically created and screened or their sequence devised in silico. In an embodiment, an aptamer library is screened against one or more targets of interest. For example, an RNA aptamer can be generated against whole cells using a cell based SELEX method. (See, e.g., Shangguan, et al., Proc. Natl. Acad. Sci. USA 103:11838-11843 (2006); which is incorporated herein by reference).

In an embodiment, the implantable device 102 includes one or more sensor components 204 having at least one targeting moiety that binds a biomarker associated with a neuropsychiatric disorder. For example, in an embodiment, the one or more sensor components 204 include at least one peptide-based aptamer that binds one or more biomarkers associated with a neuropsychiatric disorder. Non-limiting examples of peptide-based aptamers include artificial proteins where inserted peptides are expressed as part of a primary sequence of a structurally stable protein or scaffold. (See, e.g., Crawford et al., Peptide Aptamers: Tools for Biology and Drug Discovery, Briefings in Functional Genomics and Proteomics, 2 (1): 72-79 (2003); which is incorporated herein by reference).

In an embodiment, the targeting moieties 304 include one or more synthetic small molecule compounds such as an agonist or antagonist that interact with a target on, or in proximity to, a cell or tissue. Non-limiting examples of agonists, antagonists, or other small molecule compounds include those approved by the U.S. Food and Drug Administration (FDA) for use in humans such as, for example, those listed in Remington: The Science and Practice of Pharmacy, 21st Edition, 2005, edited by David Troy, Lippincott Williams & Wilkins, Baltimore Md.

In an embodiment, the targeting moieties 304 include one or more lectins. Non-limiting examples of lectins include agglutinins that could discriminate among types of red blood cells and cause agglutination, sugar-binding proteins from many sources regardless of their ability to agglutinate cells, or the like. Lectins have been found in plants, viruses, microorganisms and animals. Because of the specificity that each has toward a particular carbohydrate structure, even oligosaccharides with identical sugar compositions can be distinguished or separated. Some lectins will bind only to structures with mannose or glucose residues, while others can recognize only galactose residues. Some lectins require that a particular sugar be in a terminal non-reducing position in the oligosaccharide, while others can bind to sugars within the oligosaccharide chain. Some lectins do not discriminate between a and b anomers, while others require not only the correct anomeric structure but also a specific sequence of sugars for binding.

Further non-limiting examples of lectins include algal lectins (e.g., b-prism lectin); animal lectins (e.g., tachylectin-2, C-type lectins, C-type lectin-like, calnexin-calreticulin, capsid protein, chitin-binding protein, ficolins, fucolectin, H-type lectins, 1-type lectins, sialoadhesin, siglec-5, siglec-7, micronemal protein, P-type lectins, pentrxin, b-trefoil, galectins, congerins, selenocosmia huwena lectin-I, Hcgp-39, Ym1); bacterial lectins (e.g., Pseudomonas PA-IL, Burkholderia lectins, chromobacterium CV-IIL, Pseudomonas PA IIL, Ralsonia RS-ILL, ADP-ribosylating toxin, Ralstonia lectin, Clostridium hemagglutinin, botulinum toxin, tetanus toxin, cyanobacterial lectins, FimH, GafD, PapG, Staphylococcal enterotoxin B, toxin SSL11, toxin SSL5); fungal and yeast lectins (e.g., Aleuria aurantia lectin, integrin-like lectin, Agaricus lectin, Sclerotium lectin, Xerocomus lectin, Laetiporus lectin, Marasmius oreades agglutinin, agrocybe galectin, coprinus galectin-2, Ig-like lectins, L-type lectins); plant lectins (e.g., alpha-D-mannose-specific plant lectins, amaranthus antimicrobial peptide, hevein, pokeweed lectin, Urtica dioica UD, wheat germ WGA-1, WGA-2, WGA-3, artocarpin, artocarpus hirsute AHL, banana lectin, Calsepa, heltuba, jacalin, Maclura pomifera MPA, MornigaM, Parkia lectins, abrin-a, abrus agglutinin, amaranthin, castor bean ricin B, ebulin, mistletoe lectin, TKL-1, cyanovirin-N homolog, and various legume lectins); or viral lectins (e.g., capsid protein, coat protein, fiber knob, hemagglutinin, and tailspike protein) (see, e.g., E. Bettler, R. Loris, A. Imberty 3D-Lectin database: A web site for images and structural information on lectins, 3rd Electronic Glycoscience Conference, The internet and World Wide Web, 6-17 Oct. 1997; http://www.cermav.cnrs.fr/lectines/).

In an embodiment, the targeting moieties 304 include one or more synthetic elements such as an artificial antibody or other mimetic. Examples of synthetic elements can be found in, for example, the following documents: U.S. Pat. No. 5,804,563 (issued Sep. 8, 1998); U.S. Pat. No. 5,831,012 (issued Nov. 3, 1998); U.S. Pat. No. 6,255,461 (issued Jul. 3, 2001); U.S. Pat. No. 6,670,427 (issued Dec. 30, 2003); U.S. Pat. No. 6,797,522 (issued Sep. 28, 2004); U.S. Patent Pub. No. 2004/0018508 (published Jan. 29, 2004); Ye and Haupt, Anal Bioanal Chem. 378: 1887-1897 (2004); and Peppas and Huang, Pharm Res. 19: 578-587 (2002); each of which is incorporated herein by reference. Further non-limiting examples of antibodies, recognition elements, or synthetic molecules that recognize a cognate include those available from commercial source such as Affibody®. (See, e.g., Affibody® affinity ligands (Abcam, Inc. Cambridge, Mass. 02139-1517; U.S. Pat. No. 5,831,012 (issued Nov. 3, 1998); each of which is incorporated herein by reference).

In an embodiment, the targeting moieties 304 include one or more artificial binding substrates formed by, for example, molecular imprinting techniques and methodologies. A more detailed discussion of molecular imprinting can be found in, for example, the following documents: U.S. Pat. No. 7,442,754 (issued Oct. 28, 2008), U.S. Pat. No. 7,288,415 (issued Oct. 30, 2007), U.S. Pat. No. 6,660,176 (issued Dec. 9, 2003), and U.S. Pat. No. 5,801,221 (issued Sep. 1, 1998); each of which is incorporated herein by reference. In an embodiment, a target template is combined with functional monomers which, upon cross-linking, forms a polymer matrix that surrounds the target template. Removal of the target template leaves a stable cavity in the polymer matrix that is complementary in size and shape to the target template. As such, functional monomers of a polymer-forming matrix such as acrylamide and ethylene glycol dimethacrylate, for example, can be mixed with one or more target template in the presence of a photoinitiator such as 2,2-azobis(isobutyronitrile). The monomers can be cross-linked to one another using ultraviolet irradiation. The resulting polymer can be crushed or ground into smaller pieces and washed to remove the one or more target template, leaving a particulate matrix material capable of binding one or more target. Examples of other functional monomers, cross-linkers and initiators useful to generate an artificial binding substrate have been described elsewhere (see, e.g., U.S. Pat. No. 7,319,038 (issued Jan. 15, 2008); which is incorporated herein by reference).

In an embodiment, the one or more sensor components 204 include a biological molecule capture layer 302 having at least one targeting moiety 304 directed to gene expression products. For example, in an embodiment, a targeting moiety 304 can specifically target a gene, an mRNA, a microRNA, a gene product, a protein, a glycosylation of a gene product, a substrate or metabolite of a gene product, or the like. (See, e.g., U.S. Patent Publ. No. 2008-0206152 (published Aug. 28, 2008); which is incorporated herein by reference). Examples of targeting moieties 304 that bind RNA or DNA include microRNA, anti-sense RNA, small interfering RNA (siRNA), anti-sense oligonucleotides, protein-nucleic acids (PNAs). In an embodiment, one or more targeting moieties 304 are configured to target a compound directly associated with gene expression (e.g., transcription factors, acetylated histones, zinc finger proteins, translation factors, a metabolite of an enzyme, or the like).

In an embodiment, the targeting moieties 304 are configured to target an in vivo component in, on, or outside a cell. Non-limiting examples of in vivo targets include carbohydrates, cell surface proteins (e.g., cell adhesion molecules, cell surface polypeptides, membrane receptors, or the like), cytosolic proteins, intracellular components (e.g., one or more components of a signaling cascade such as, for example, one or more signaling molecules, kinases, phosphatases, transcription factors, signaling peptides, signaling proteins, or the like), metabolites, nuclear proteins, receptors, or secreted proteins (e.g., growth factors, cell signaling molecules, or the like). In an embodiment, the one or more sensor components 204 include an array of micro-regions modified to capture target molecules. For example, in an embodiment, the one or more sensor components 204 include array of micro-regions that incorporate one or more targeting moieties 304 that selectively target one or more materials, substances, chemicals, components, biomarkers, or the like indicative of mental disorders, disease states, pathological conditions, or the like. Referring to FIG. 3B, in an embodiment, the system 100 includes an implantable device 102 having body structure 104 configured to sufficiently internally reflect at least a portion of an emitted energy stimulus 308 and to generate an evanescent field 310 across one or more regions of the body structure 104. In an embodiment, at least a portion of the body structure 104 includes one or more energy waveguides configured to sufficiently internally reflect at least a portion of an emitted energy stimulus 308 and to generate an evanescent field 310.

Evanescent fields 310 can be generated, for example, via diffraction from a grating or a collection of apertures; scattering from an aperture; or total internal reflection at the interface between two media See e.g., Smith et. al, Evanescent Wave Imaging in Optical Lithography, Proc. SPIE 6154, (2006). For example, electromagnetic energy 308 crossing a boundary 312 between materials with different refractive indices (nᵢ), partially refracts at the boundary surface, and partially reflects. When the incident angle (θᵢ), exceeds the critical angle of incidence, define as:

$${θ}_{critical} = {sin}^{- 1} ( \frac{{n}_{low}}{{n}_{high}} ) ,$$

the electromagnetic energy traveling from a medium of higher refractive index (n_{high}) to that of a lower one (n_{low}), undergoes total internal reflection (see e.g., FIG. 3), and generates an evanescent field 310 near the boundary 312 (the intensity of which decays exponentially with increasing distance from the surface). In an embodiment, at least a portion of the body structure 104 is configured to sufficiently internally reflect at least a portion of an emitted energy stimulus 308 to cause an evanescent electromagnetic field 310 to emanate from at least a portion of the body structure 104. In an embodiment, at least a portion of the body structure 104 is configured to internally reflect at least a portion of an emitted energy stimulus 302 within an interior of at least one of the one or more fluid-flow passageways 108. In an embodiment, at least a portion of the body structure 104 is configured to totally internally reflect at least a portion of an emitted energy stimulus 308 propagated within an interior of at least one of the one or more fluid-flow passageways.

In an embodiment, adsorbing molecules 308 cause changes in the local index of refraction, resulting in changes in the resonance conditions of the evanescent electromagnetic field 310. In an embodiment, detected index of refraction changes are correlated to the presence of one or more target markers 306.

In an embodiment, an implantable device 102 includes a sensor component 204 having one or more surface-plasmon-resonance (SPR) based sensors for detecting captured target molecules. In an embodiment, the SPR based sensors detect target molecules suspended in a fluid by reflecting light off thin metal films in contact with the fluid. Adsorbing molecules cause changes in the local index of refraction, resulting in detectable changes in the resonance conditions of the surface plasmon waves. In an embodiment, the one or more sensor components 204 include at least one SPR microarray sensor having an array of micro-regions modified to capture target molecules. In an embodiment, an SPR microarray sensor is configured to detect refractive index changes indicative of a change in a level of captured targeted molecules. In an embodiment, a multiplex method includes identifying one or more factors associated with a specific disease states, pathologies, or conditions by targeting with one or more targeting moieties.

In an embodiment, the one or more sensor components 204 are configured to detect one or more factors associated with a specific disease state, pathology, or the like. For example, in an embodiment, the one or more sensor components 204 include a substrate having one or more targeting moieties 304 that selectively target at least one of a specific tissue, cell, genomic target, biological target, chemical target, or the like associated with a specific disease state, pathology, or the like. The binding of the targeting moieties 304 to respective targets causes changes to the local index of refraction, which are detectable by the one or more sensor components 204.

In an embodiment, at least one of the one or more sensor components 204 is configured to detect the presence or concentration of specific target chemicals (e.g., marker, blood components, biological fluid component, cerebral spinal fluid component, infectious agents, infection indication chemicals, inflammation indication chemicals, diseased tissue indication chemicals, biological agents, molecules, ions, or the like).

In an embodiment, the system 100 includes an implantable device 102 configured to detect a formation or presence of a pathological condition associated with a suicidal tendency. For example, in an embodiment, an elevated risk for suicide is assessed by monitoring pathological conditions (e.g., biomarkers levels) in CSF. Non-limiting examples of pathological conditions or biomarkers associated with a suicidal tendency include increased levels of corticotropin-releasing hormone and decreased levels of biogenic amine, e.g., norepinephrine, serotonin (5-HT), homovanillic acid (HVA), and 5-hydroxyindole acetic acid (5-HIAA). In an embodiment, a presence of a pathological condition associated with a suicidal tendency is inferred from changes to a local index of refraction caused by monoclonal antibody associating with corticotropin-releasing hormone on a sensor surface.

In an embodiment, detected decreased levels of 5-hydroxyindole-acetic acid (5-HIAA) in CSF can be indicative of an episode of major depression or of an elevated risk for suicide (see, e.g., Mann, et al., Neuropsychopharmacology, 15:576-586, 1996; which is incorporated herein by reference). Dysfunction of the central monoaminergic system appears to play a critical role in depression and suicidal tendency. In an embodiment, an increased HVA/5-HIAA ratio can be indicative of impaired serotonergic modulation of dopamine activity and altered relationships between the monoamine metabolites have been proposed to be associated with suicidal tendency. In an embodiment, HVA/5-HIAA ratios that are approximately 50% those of normal subjects can be indicative of an elevated risk for suicide. (See, e.g., Jokinen, et al., Arch. Suicide Res., 11:187-192, 2007; which is incorporated herein by reference).

In an embodiment, decreasing levels of the noradrenaline metabolite 3-methoxy-4-hydroxyphenylglycol (MHPG) in CSF are correlated with increasing suicidal tendencies (e.g., an approximately 22% increase in hazard for each 10 μmol/ml lower MHPG). Subjects at or above a median cut off of about 45 μmol/ml MHPG are less likely to attempt suicide than those subjects with MHPG levels below about 45 μmol/ml. (See, e.g., Galfalvy, et al., Int J. Neruopsychopharmacol. 12:1327-1335, 2009; which is incorporated herein by reference). In an embodiment, the lower the level of MHPG in cerebrospinal fluid, the more medically lethal the future suicide attempt. Smoking and self-rated severity of depression are also associated with lower levels of MHPG in cerebrospinal fluid and with suicidal tendencies. In an embodiment, the level of MHPG in CSF is associated with short-term risk for future suicidal behavior in the 12 months following a major depressive episode. (See, e.g., Galfalvy, et al., Int J Neruopsychopharmacol. 12:1327-1335, 2009; which is incorporated herein by reference).

In an embodiment, the implantable device 102 is configured to detect proinflammatory biomarkers in the cerebrospinal. In an embodiment, assessment of proinflammatory biomarkers in CSF are used alone or in combination with other biomarkers to identify suicidal tendency. For example, in an embodiment, a detected elevated interleukin-6 (IL-6) level in CSF compared with normal controls is indicative of an elevated risk for suicide. In an embodiment, increasing levels of IL-6 in CSF is positively correlated with decreasing levels of HVA and 5-HIAA and increasing severity of depressive symptoms. In an embodiment, an approximate six-fold increase in IL-6 in CSF is correlated with violent suicide attempts versus non-violent suicide attempts. (See, e.g., Lindqvist, et al., Biol. Psychiatry, 66:287-292, 2009; which is incorporated herein by reference).

In an embodiment, the implantable device 102 is configured to detect orexins in CSF. Assessment of orexins in CSF can be used alone or in combination with other biomarkers to identify suicidal tendency. Orexins are neuropeptides secreted from the lateral hypothalamus that have an important role in the regulation of wakefulness. Subjects with suicidal tendencies and major depressive disorder have lower levels of orexin in CSF than other subject groups and low orexin levels are associated with severe symptoms of lassitude and decreased motor function. Subjects who are perceived by a psychiatrist as more globally ill display the lowest levels of orexin in CSF. In an embodiment, a detected increase orexin (from approximately 160 pg/ml to approximately 183 pg/ml) can be indicative of a past elevated risk for suicide. (See, e.g., Brundin, et al., J Affect. Dis., 113:179-182, (2009); which is incorporated herein by reference).

While human males with schizophrenia spectrum psychosis with prior suicide attempt are at a high risk for suicide, there does not appear to be a correlation between CSF monoamine metabolites levels (HVA and HIAA) and suicidal behavior in this population. As such, suicidal behavior in schizophrenia spectrum psychosis, in contrast to mood disorders, may not be readily predicted by levels of 5-HIAA and HVA in CSF. (See, e.g., Carlborg, et al., Schizophrenia Res. 112:80-85, (2009); which is incorporated herein by reference).

In an embodiment, the system 100 includes an implantable device 102 configured to detect a formation or presence of a pathological condition associated with psychosis. For example, in an embodiment, the system 100 includes an implantable device 102 configured to detect one or more markers associated with psychosis. Psychosis includes neuropsychiatric disorder that markedly interfere with a subject's capacity to meet life's everyday demands and more specifically refers to a thought disorder in which reality testing is grossly impaired, characterized by delusions and hallucinations. Symptoms can include disorganized thought and speech; seeing, hearing, smelling, or tasting things that are not there (hallucinations); paranoia; and delusional thoughts. Depending on the condition underlying the psychotic symptoms, symptoms can be constant or may come and go. Psychosis can occur as a result of alcohol or drug abuse, brain tumors or cysts, dementia (including Alzheimer's disease), degenerative brain diseases (e.g., Parkinson's disease, Huntington's disease), certain chromosomal disorders, HIV and other infections of the brain, some types of epilepsy, or stroke. Psychosis is also a component of a number of psychiatric disorders, including bipolar disorder, delusional disorder, depression with psychotic features, personality disorder (e.g., schizotypal, schizoid, paranoid, and sometimes borderline), schizoaffective disorder, or schizophrenia.

A number of neuropsychiatric disorders, including schizophrenia, depression, or suicidal tendency are correlated with changes in the levels of dopamine and serotonin metabolites in CSF. The most prominent of these are homovanillic acid (HVA), a metabolite of dopamine and 5-hydroxyindoleacetic acid (5-HIAA), a metabolite of serotonin. In an embodiment, the severity of psychosis is assed from detected increases in the levels of HVA CSF. In an embodiment, a 3-4 fold (e.g., from about 20 ng/ml to about 100 ng/ml) increase in the levels of HVA in CSF is indicative of a severe psychosis disease state. (See, e.g., Maas, et al., Schizophrenia Bulletin, 23:147-154, (1997); which is incorporated herein by reference). Conversely, the levels of 5-HIAA are reduced in CSF of subjects exhibiting anti-social behavior with the largest reductions associated with alcoholism/alcohol abuse, suicide attempts, and with subjects 30 years of age or younger (see, e.g., Moore, et al., Aggressive Behavior, 28:299-316, (2002); which is incorporated herein by reference). In an embodiment, 5-HIAA levels in CSF are a strong correlate of current and future suicidal behavior.

Subjects with a history of a suicide attempt have lower levels of 5-HIAA in CSF across diagnoses of depression, schizophrenia, or personality disorders compared with psychiatrically matched control groups. In an embodiment, changes in the levels of HVA and 5-HIAA are used to track a response to antipsychotic medication. For example, treatment with the atypical antipsychotic drug olanzapine significantly increases the levels of HVA in CSF whereas the levels of 5-HIAA remain relatively unchanged (see, e.g., Scheepers, et al., Neuropsychopathology 25:468-475, (2001); which is incorporated herein by reference).

Non-limiting examples of pathological conditions or biomarkers associated with psychosis include increased levels of HVA or VGF nerve growth factor, and decreased levels of transthyretin. For example, in an embodiment, an approximate threefold increase in CSF level of a VGF-derived fragment (approximately 4000 daltons) relative to normal control levels is indicative of first onset paranoid schizophrenia in drug-naïve subjects. In an embodiment, a twofold decrease in transthyretin, a thyroid hormone binding protein, relative to normal control levels is indicative of first onset paranoid schizophrenia in drug-naïve subjects. (See, e.g., Huang, et al., PLoS Medicine, 3(11):e428, doi:10.1371/journal.pmed.0030428 (2006); which is incorporated herein by reference). In an embodiment, an implantable device 102 generates a disease state indicative of psychosis from a detected increase of VGF fragment and a detected decrease of transthyretin in CSF.

In an embodiment, the system 100 includes an implantable device 102 configured to differentiate between various neuropsychiatric disorders. In an embodiment, detected biomarkers levels are used to differentiate among causes of psychosis, e.g., schizophrenia versus Alzheimer's disease. For example, in an embodiment, relative changes in detected levels of VGF nerve growth factor and transthyretin are used to differentiate between various neuropsychiatric disorders. In an embodiment, paranoid schizophrenia is correlated with increased levels of VGF nerve growth factor and decreased levels of transthyretin relative to normal controls. (See, e.g., Huang, et al., PLoS Med. 3:e428, doi:10.1371/journal.pmed.0030428 (2006); which is incorporated herein by reference). Depression is correlated with increased levels of VGF with no changes in transthyretin relative to controls. No significant changes in VGF and transthryetin are noted in subjects with Alzheimer's disease relative to normal controls.

Non-limiting examples of biomarkers of psychosis include nerve growth factor (NGF) or brain-derive neurotrophic factor (BDNF). NGF plays a role in development and functioning of cholinergic neurons in the central nervous system, while BDNF has emerged as a key contributor to brain plasticity and cognition. In an embodiment, an approximately 50% decline in the levels of NGF or BDNF in CSF is associated with first-episode psychosis in drug naïve subjects. (See, e.g., Kale, et al., Schizophr. Res. 115:209-214, 2009; Pillai, et al., Int. J Neuropsychopharmacol., 13:535-539, (2010); each of which is incorporated herein by reference).

Further non-limiting examples of biomarkers for psychosis include 5-hydroxyindoleacetic acid, anandamide, angiotensin converting enzyme, apolipoprotein A1, docohexanoic acid, D-serine, glutamate, glutamine, glycine, glycogen synthase kinase 3 beta, homovanillic acid, inositol monophosphatase, interleukin 6, kynurenic acid, neural cell adhesion molecule, nitrate, nitric oxide, nitrite, orexin A, peptide YY, phospho-tau protein, S100 calcium binding protein B, synaptosome-associated protein (SNAP-25), tau protein, thyrotropin-releasing hormone, transthyretin, and Vgf (non-acronymic). In an embodiment, an increase in a measured level of one or more of, angiotensin converting enzyme, anandamide, neural cell adhesion molecule, docohexanoic acid, glutamine, homovanillic acid, interleukin 6, inositol monophosphatase, kynurenic acid, S100 calcium binding protein B, synaptosome-associated protein of (SNAP-25), thyrotropin-releasing hormone, and Vgf (non-acronymic); and a decrease in a measured level of one or more of 5-hydroxyindoleacetic acid, apolipoprotein A1, D-serine, glutamate, glycine, glycogen synthase kinase 3 beta, nitrate, nitric oxide, nitrite, orexin A, peptide YY, and transthyretin, transthyretin (TRR) can be indicative of a diagnosis of psychosis. In an embodiment, a detected change in one or more of the markers associated with a psychosis, while the level of tau-phosphorylated or the total amount of tau protein remains the same, can be indicative of a diagnosis of psychosis.

In an embodiment, the system 100 includes an implantable device 102 configured to detect a formation or presence of a pathological condition associated with at least one of schizophrenia, bipolar disorder, or depression. For example, in an embodiment, the system 100 includes an implantable device 102 that detects one or more markers associated with at least one of schizophrenia, bipolar disorder, or depression.

Schizophrenia is a relatively common, chronic, and disabling neuropsychiatric disorder with a complex genetic and environmental basis and phenotype. Evidence suggests association between schizophrenia and brain structural abnormalities including a decrease in brain tissue volume and an increase in the volume of the CSF. (See, e.g., Crespo-Facorro, et al., Schizophr. Res., 115:191-201, (2009); which is incorporated herein by reference). In an embodiment, a measured increase in the volume of CSF is indicative of schizophrenia.

Non-limiting examples of pathological conditions or biomarkers associated with schizophrenia that are detectable in cerebral spinal fluid include increased levels of one or more of docohexanoic acid, endogenous cannibinoid anandamide, S100β, kynurenic acid, glutamine/glutamate ratio, SNAP-25, IL-6, cleaved N-CAM (cN-CAM), thyrotropin-releasing hormone (TRH), HVA, macrophages, inositol monophosphatase, angiotensin-converting enzyme (ACE), or VGF nerve growth factor. Further non-limiting examples of pathological conditions or biomarkers associated with schizophrenia that are detectable in cerebral spinal fluid include decreased levels of one or more of D-serine, glutamate, glycine, GSK-3beta, nitric oxide (NO), nitrite, nitrate, orexin A (hypocretin-1), 5-HIAA, peptide YY, transthyretin (carrier of thyroid hormone thyroxine), or apolipoprotein A1. For example, in an embodiment, a reduction (e.g., a 35% reduction) in CSF levels of apolipoprotein A1 is indicative of first-onset schizophrenia in drug naïve subjects., (See, e.g., Huang, et al., Mol. Psych., 13:1118-1128, (2008); which is incorporated herein by reference).

By comparison to schizophrenia, bipolar disorder is a neuropsychiatric disorder characterized by alternating moods of mania and depression, either of which can last weeks to months. The manic state is characterized by high energy, extreme euphoria or optimism to the point of impairing judgment, hyperactivity, decreased inhibitions, and in some subjects, delusions of grandeur. The depressive state is characterized by hopelessness, sadness, loss of interest in normal activities, lethargy, and lack of motivation.

In an embodiment, the implantable device 102 is configured to assess a condition associated with mitochondrial dysfunction by monitoring a level of lactate in CSF. Mitochondrial dysfunction can be involved in the pathophysiology of bipolar disorder and schizophrenia as indicated by histological abnormalities in mitochondrial structure, abnormal expression of mitochondrial proteins and genes, and metabolic abnormalities. In an embodiment, the implantable device 102 is configured to assess a condition associated with a schizophrenic disease state or a bipolar disorder disease state. For example, in an embodiment, an elevated lactate level, relative to normal controls, in CSF is indicative of a schizophrenic disease state or a bipolar disorder disease state.

In an embodiment, an approximate 23% higher lactate level, relative to normal controls, in CSF is indicative of a schizophrenic disease state or a bipolar disorder disease state. For example, in an embodiment, an approximate 34% higher lactate level in CSF, relative to normal controls, is indicative of bipolar disorder disease state. In an embodiment, lactate levels in CSF above a normal cut-off value of about 1.75 mM are positively correlated with bipolar disorder and schizophrenia disease states. Elevated levels of glucose in CSF (10-20% higher) relative to levels in normal controls are also correlated with bipolar disorder and schiiophrenia. In general, this suggests increased extra-mitochondrial, anaerobic glucose metabolism consistent with impaired mitochondrial function. (See, e.g., Regenold, et al., Biol. Psych., 65:489-494, 2009; Holmes, et al., PLoS Med., 3(8):e327, (2006); each of which is incorporated herein by reference).

In an embodiment, disease specific biomarkers in CSF, a number of which are described herein, can be used in combination with kynurenic acid to differentiate schizophrenia and bipolar disorder from other neuropsychiatric disorders. For example, in an embodiment, a detected increase in the cerebrospinal levels of kynurenic acid of about 50% can be indicative of bipolar disorder and schizophrenia. (See, e.g., Olsson, et al., J. Psychiatry Neurosci., 35:195-199, 2010; which is incorporated herein by reference). Elevated levels of kynurenic acid are also correlated with cerebral malaria, HIV infection, Down's syndrome, amyotrophic lateral sclerosis, and epilepsy while decreased levels of kynurenic acid are correlated with various neurodegenerative diseases, e.g., Alzheimer's disease, Parkinson's disease, multiple sclerosis, Huntington's disease as well as neonatal asphyxia. (See, e.g., Han, et al., Cell. Mol. Life. Sci., 67:353-368, (2010); which is incorporated herein by reference). Subjects with major depressive disorder or depression primarily exhibit the depressive state symptoms described above and do not oscillate between the manic and depressive state symptoms associated with bipolar disorder.

Non-limiting examples of pathological conditions or biomarkers associated with depression include increased levels of Aβ42 (in elderly women) and decreased levels of transthyretin, 5-HIAA, serotonin, or neuropeptide Y. The diagnosis of major depressive disorder in elderly women is accompanied by increased levels of amyloid beta-42 (Aβ42), a biomarker normally reduced in CSF of elderly subjects with Alzheimer's disease. Increased levels of Aβ42 in CSF in combination with a higher CSF/serum albumin ratio, is indicative of neuropathological and vascular factors in depressed elderly women that differ from what is observed in aged match subjects with Alzheimer's disease. (See e.g., Gudmundsson, et al., Psychiatry Res., 176:174-178, (2010); which is incorporated herein by reference).

Depression is also associated with decreased CSF levels of transthyretin, a thyroid hormone-binding protein produced by the choroid plexus and secreted into the CSF. The median level of transthyretin in CSF of depressed subjects (about 4.4 mg/liter; range from about 2-7 mg/liter) is reduced by approximately 40% relative to the median levels in normal subjects (about 7.3 mg/liter; range from about 2-20 mg/liter). (See, e.g., Sullivan, et al., Am. J. Psychiatry, 156:710-715, 1999; which is incorporated herein by reference). In an embodiment, a detected increase in Aβ42 levels and a decrease in transthyretin levels in CSF is indicative of major depressive disorder, particularly in elderly females.

Non-limiting examples of biomarkers for schizophrenia include 5-hydroxyindoleacetic acid, angiotensin-converting enzyme (ACE), Apolipoprotein A1 cleaved N-CAM (cN-CAM), docohexanoic acid, d-serine, endogenous cannibinoid anandamide, glutamate, glutamine, glycine, glycogen synthase kinase 3 beta, homovanillic acid, inositol, interleukin 6, kynurenic acid, macrophages, monophosphatase, nerve growth factor inducible (Vgf) nitrate, nitric oxide (NO), nitrite, orexin A (hypocretin-1), peptide YY, S100B, synaptosome-associated protein (SNAP-25), thyrotropin-releasing hormone (TRH), or transthyretin. In an embodiment, an increase in a measured level of one or more of angiotensin-converting enzyme (ACE), cleaved N-CAM (cN-CAM), docohexanoic acid, endogenous cannibinoid anandamide, glutamate, glutamine, homovanillic acid, inositol, interleukin 6, kynurenic acid, macrophages, monophosphatase, S100B, synaptosome-associated protein (SNAP-25), thyrotropin-releasing hormone (TRH), or Vgf; and a decrease in a measured level of one or more of 5-hydroxyindoleacetic acid, apolipoprotein A1 d-serine, glutamate, glycine, glycogen synthase kinase 3 beta, nitrate, nitric oxide (NO), nitrite, orexin A (hypocretin-1), peptide YY, or transthyretin can be indicative of a diagnosis of schizophrenia.

In an embodiment, the system 100 includes an implantable device 102 configured to detect a formation or presence of a pathological condition associated with a major depressive disorder. For example, in an embodiment, the implantable device 102 includes at least one sensor component 204 including a biomarker capture layer having one or more antibodies that specifically bind to one or more biomarkers indicative of major depressive disorder. In an embodiment, the sensor component 204 includes a biomarker capture layer having a monoclonal antibody that specifically binds to corticotropin-releasing hormone (e.g., clone 2B11, product number WH0001392M2, Sigma-Aldrich, St. Louis, Mo.).

In an embodiment, the system 100 includes an implantable device 102 configured to detect a formation or presence of a pathological condition associated with a neuropsychiatric disorder. For example, in an embodiment, the system 100 includes an implantable device 102 configured to detect one or more markers associated with a neuropsychiatric disorder. Neuropsychiatric disorders include behavioral disorders with concomitant and demonstrable pathologies within the central nervous system. Non-limiting examples of neuropsychiatric disorders include dementia, brain injury, and cognitive processing disorders, as well as psychiatric manifestations associated with neurological disorders including epilepsy, cerebrovascular accidents, and movement and degenerative disorders. Further non-limiting examples of neuropsychiatric disorders include Alzheimer's disease, Parkinson's disease, Huntington's disease, Pick's disease, Wilson's disease, Epilepsy, traumatic brain injury, cerebral vascular disease, brain tumors, multiple sclerosis, autism, narcolepsy, prion diseases and various mental illnesses including, among others, depression, schizophrenia, bipolar disorder, panic disorder, obsessive compulsive disorder, and eating disorders. (See, e.g., Taber, et al., Annu. Rev. Med., 61:121-133, (2010); which is incorporated herein by reference).

In an embodiment, neuropsychiatric disorders are categorized based on the type of condition producing the observed or measured neuropsychiatric symptoms. These types of conditions include, for example, developmental (e.g., Autism, Down's syndrome, seizure disorders, etc.); degenerative (e.g., Alzheimer's disease, amyotrophic lateral sclerosis, central pontine myelinolysis, Huntington's disease, Parkinson's disease, Pick's disease, etc.); metabolic (e.g., adrenal cortex disease, B12 deficiency, electrolyte imbalance, hypoglycemia, hypothyroidism, hypoxia, parathyroid disease, thiamine deficiency, uremia, and Wilson's disease, poisons/toxins, e.g., carbon monoxide, cyanide, heavy metals, methanol, organophosphates, solvents, intoxication or withdrawal, hallucinogens, opiates, stimulants, sedatives, hypnotics, etc); general medical conditions (e.g., infections (e.g., hepatitis, AIDS, brain abscess, prion disease, viral encephalitis, meningitis, toxoplasmosis, urinary tract infection)); neoplasms (e.g., glioma, hypothalamic hamartoma, meningioma, pituitary tumors, metastatic tumors); traumas (e.g., closed head injury, postoperative damage, axonal shearing injury, subdural hematomas); immune (e.g., multiple sclerosis, systemic lupus erythematosus); or organ failure (e.g., chronic obstructive pulmonary disease, congestive heart failure, hepatic encephalopathy, and renal failure). (See, e.g., Taber, et al., Ann. Rev. Med., 61:121-133, (2010); which is incorporated herein by reference).

In an embodiment, the implantable device 102 is configured to detect changes in biomarkers in CSF associated with a neuropsychiatric disorder. For example, in an embodiment, the implantable device 102 includes one or more sensors 206 having an array of micro-regions modified with one or more monoclonal antibodies specific for a neuropsychiatric disorder biomarker.

In an embodiment, the system 100 includes an implantable device 102 configured to detect a formation or presence of a pathological condition associated with dementia. For example, in an embodiment, the system 100 includes an implantable device 102 configured to detect one or more markers associated with dementia includes, among other things, a progressive decline of cognitive function due to damage or disease in the brain beyond that associated with the normal aging process. Alzheimer's disease (Aβ) is the most common form of dementia with approximately 50% of dementia subjects suffering from the disease. However, there are a number of other types of dementia including other cortical dementias, e.g., vascular dementia, dementia with Lewy bodies, frontotemporal dementia, and Creutzfeldt-Jakob disease (CJD) and subcortical dementias, e.g., dementia due to Huntington's disease, hypothyroidism, Parkinson's disease, vitamin deficiency, syphilis, hypercalcemia, hypoglycemia, AIDS, and other pathologies. In an embodiment, analysis of components of the CSF is used to differentiate between various forms of dementia. (See, e.g., Knopman, et al., Neurology 56:1143-1153, (2001); which is incorporated herein by reference).

In an embodiment, the system 100 includes an implantable device 102 configured to detect a formation or presence of a pathological condition associated with human immunodeficiency virus (HIV) associated dementia. For example, in an embodiment, the system 100 includes an implantable device 102 configured to detect one or more markers associated with HIV-associated dementia.

Central nervous system infection is a nearly uniform feature of untreated human immunodeficiency virus type 1 (HIV-1) infection, with HIV-1 viral RNA detected to varying degrees in CSF of most subjects not undergoing treatment with combination antiretroviral therapy. In its chronic phase, CNS infection is not necessarily accompanied by neurological symptoms or signs, but can progress into more invasive HIV encephalitis (HIVE) that manifests clinically as HIV-associated dementia (also termed AIDS dementia complex). Cognitive, behavioral, and motor dysfunction induced as a consequence of HIV-1 infection remain chronic and debilitating despite the advent of antiretroviral therapy. In its most severe form, HIV-associated cognitive impairment is termed HIV-associated dementia. Anti-retroviral therapy has altered the magnitude of HIV-associated cognitive impairments as subjects present milder forms of disease and show different patterns of mental dysfunction.

Antiretroviral therapy has also prolonged the life expectancy of HIV-infected subjects such that HIV-associated cognitive impairments need to be differentiated from age-related neurodegenerative diseases, e.g., Alzheimer's disease. In an embodiment, the system 100 includes an implantable device 102 configured to differentiate between HIV-associated dementia and Alzheimer's disease by comparing detected levels of biomarker in CSF to reference filtering information 234. Several biomarkers and pathological conditions in CSF such as, for example, soluble amyloid precursor protein alpha (sAPPα) and beta (sAPPβ), Aβ42, and total, and phosphorylated tau can be used to differentiate between HIV-associated dementia and Alzheimer's disease. For example, in an embodiment, a detected decrease in the CSF levels of sAPPα and sAPPβ (e.g., 50% reduction), a slight increase in the levels of total tau, and no change in the levels of phosphorylated-tau can be indicative of HIV-associated dementia as compared with Alzheimer's disease. (See, e.g., Gisslen, et al., BMC Neurology, 9:63, (2009); which is incorporated herein by reference).

In an embodiment, the system 100 includes an implantable device 102 configured to diagnose cognitive impairment in HIV subjects by comparing detected levels of biomarker in CSF to reference filtering information 234. Non-limiting examples of pathological conditions or biomarkers for HIV-associated dementia include increased levels of nerve growth factor, cysteine histidine-rich (PINCH) protein, CSF HIV-1 RNA, neopterin (indicative of inflammation), light chain neurofilament (NFL), ceramide, 4-hydroxynonenals, tau, glial fibrillary acidic protein (GFAP), chemokine (C-X-C motif) ligand 10 (CXCL10), urokinase-type plasminogen activator (uPA), monocyte chemoattractant protein (MCP)-1, microglial marker (mI), and decreased levels of soluble amyloid precursor proteins, fibroblast growth factor (FGF-2), or brain-derived neurotrophic factor (BDNF). Non-limiting examples of biomarkers for HIV-associated dementia include 4-hydroxynonenals, acyiphosphatase 1, brain-derived neurotrophic factor (BDNF), CSF HIV RNA, CXCL10, cysteine histidine-rich (PINCH) proteins, fibroblast growth factor FGF-2, galectin-7, glial fibrillary acidic protein (GFAP), light chain neurofilament (NFL), L-plastin, macrophage capping protein, microglial marker (ml), migration inhibitory factor-related rpotein14, monocyte chemoattractant protein (MCP)-1, neopterin, nerve growth factor (NGF), neurosecretory protein VGF, soluble superoxide dismutase, tau protein, tyrosine 3/tryptophan 5-monooxygenase activation protein, or urokinase-type plasminogen activator (uPA).

In addition, a number of biomarkers are detected in CSF of normal subjects that are not detected in CSF of cognitively impaired HIV positive subjects. These include, among others, apolipoprotein H, brain specific angiogenesis inhibitor 2, cell surface glycoprotein MUC18, chromogranin B precursor, extracellular superoxide dismutase, fibrinogen beta chain, MAP/ERK kinase kinase 4, N-acetyllactosaminide beta-1,3-N-acetylglucosaminyltransferase, olfactory receptor 1B1, protein FAM3C, and tyrosine-protein phosphatase non-receptor type substrate 1 precursor. (See, e.g., Laspiur, et al., J. Neuroimmunol. 192:157-170, (2007); which is incorporated herein by reference).

In an embodiment, the system 100 includes an implantable device 102 configured to diagnose cognitive impairment in HW by comparing detected levels of neopterin in CSF to reference filtering information 234. For example, in an embodiment, detected levels of inflammatory biomarker neopterin in CSF are positively correlated with both HIV infection, as well as with HIV-associated dementia. The CSF of HIV negative subjects on average contains less than 5 nmol/L neopterin, whereas the CSF of untreated HIV infected subjects without dementia and of HIV infected subjects with dementia can contain 10-25 nmol/L and 40-60 nmol/L, respectively. While neopterin is also elevated in association with opportunistic CNS infections, it can be used to assess whether an HIV positive subject has or is progressing towards HIV-associated dementia. (See, e.g., Hadberg, et al., AIDS Res. Ther. 7:15, (2010); which is incorporated herein by reference).

In an embodiment, the system 100 includes an implantable device 102 configured to diagnose HIV-associated dementia by monitoring neurofilament protein levels in CSF. For example, in an embodiment, an approximate 10 fold increase in neurofilament protein (e.g., greater than about 2000 ng/ml) in CSF relative to normal levels (e.g., less than about 250 ng/ml) is indicative of HIV-associated dementia or other CNS opportunistic infections in subjects infected with HIV. The level of neurofilament protein declines in CSF of subjects with HIV-associated dementia in response to antiretroviral treatment. (See, e.g., Abdulle, et al., J. Neurol. 254:1026-1032, (2007); which is incorporated herein by reference).

In an embodiment, the system 100 includes an implantable device 102 configured to differentiate between pathologically active HIV-associated dementia and quiescent HIV-associated dementia by comparing detected levels of biomarker in CSF to reference filtering information 234. For example, in an embodiment, detected CSF levels of HIV-1 viral RNA are used as a diagnostic tool. HIV-1 viral RNA is readily detected in CSF of nearly all asymptomatic, treatment naïve subjects with a wide range of concentrations and as such simply detecting HIV-1 viral RNA in CSF is diagnostically nonspecific for HIV-associated dementia. However, failure to detect HIV-1 viral RNA using very sensitive methods suggests that active CNS infection is not present and therefore unlikely to be the cause of any ongoing neural symptoms or damage. Further non-limiting examples of biomarkers of immune activation that can be indicative of HIV-associated dementia include beta-2-microglobulin, neopterin, quinolinic acid, or CCL2. In an embodiment, detected levels of neopterin and CCL2 in CSF are indicators of macrophage activation and chemotaxis, and thus indicate perturbation of this central pathogenic component. Both are elevated in HIV-associated dementia and elevations in the plasma:CSF ratio of CCL2 has been reported to precede HIV-associated dementia. One or more of these measurements might be of ancillary value when used along with other biomarkers. Interpreted together with the CSF levels of HIV-1 viral RNA, elevation of CCL2 and/or neopterin suggest not only that local HIV-1 infection is present but that macrophages are activated—processes that can be necessary, if not sufficient to cause HIV-associated dementia.

In an embodiment, active forms of HIV-associated dementia are distinguished from inactive forms by monitoring for biomarkers of oxidative stress (e.g., reactive aldehydes such as 4-hydroxy-nonenal). In an embodiment, viral biomarkers and immune biomarkers are combined with neural biomarkers to diagnose and assess active injury. In an embodiment, neural biomarkers (e.g., molecular products of neurons, astrocytes, oligodendrocytes, or microglia) are used in the diagnosis of HIV-associated dementia. (See, e.g., Price, et al., Neurology, 69:1781-1788, (2007); which is incorporated herein by reference).

In an embodiment, the system 100 includes an implantable device 102 configured to monitored a progression of CNS HIV infection and associated dementia by measuring the concentrations of HIV-1 viral RNA (an indicator of viral load), neopterin (an indicator of immunoactivation), and neurofilament (NFL; an indicator of CNS injury) in CSF of an HIV infected subject. (See, e.g., Gisslen, et al., J. Neuroimmune Pharmacol., 2:112-119, (2002); which is incorporated herein by reference). Briefly, benign CNS infection and immunoactivation are characterized by detectable viral RNA (>50 copies/ml), mildly elevated neopterin (<5 nmol/l) and normal NFL (<500 ng/l). Preinjury CNS immunoactivation is a state of heightened CNS immune activation characterized by increased viral RNA (>500 copies/10, elevated neopterin (>22 nmol/L), but without brain injury as indicated by normal NFL levels. Subclinical HIV-related neurodegeneration is characterized by mildly elevated NFL (>500 ng/ml) with increased viral RNA (>1000 copies/ml) and neopterin (>22 nmol/l). Lastly, overt HIV-related neurodegeneration is accompanied by high levels of NFL (>1,000 ng/l), neopterin (>22 nmol/l), and viral RNA (>1,000 copies/ml).

In an embodiment, the system 100 includes an implantable device 102 configured to detect a formation or presence of a pathological condition associated with Alzheimer's disease. For example, in an embodiment, the implantable device 102 includes at least one sensor component 204 having a biological molecule capture layer 302 incorporating one or more targeting moieties 304 that selectively target a biomarker associated with Alzheimer's disease. Alzheimer's disease is a progressive neurodegenerative disorder that causes dementia in approximately 10% of subjects aged 65 years or older. The disease is characterized by a spectrum of symptoms including the inability to acquire new memories, confusion, irritability and aggression, mood swings, language breakdown, long-term memory loss, general withdrawal as senses decline, loss of bodily functions, and ultimately death. The clinical stages of Alzheimer's disease have been divided into three phases. In the first phase, termed the pre-symptomatic phase, subjects are cognitively normal but have some pathological changes in the central nervous system associated with Alzheimer's disease. In the second phase, termed the prodromal phase or mild cognitive impairment (MCI), subjects begin to exhibit the earliest cognitive symptoms (typically deficits in episodic memory) that do not otherwise meet the criteria for dementia. The final phase in the evolution of Alzheimer's disease is dementia.

Alzheimer's disease begins with abnormal processing of amyloid precursor protein which then leads to excess production or reduced clearance of β-amyloid (Aβ) and the formation of plaques in the cortex of the brain. By an unknown mechanism, one or more isoforms of Aβ induce a cascade of events leading to abnormal aggregation of tau (a highly soluble microtubule-associated protein), synaptic dysfunction, cell death, and brain shrinkage. The deposition of Aβ-plaques in the brain associated with the early phases of Alzheimer's disease is characterized by decreasing levels of the β3-amyloid isoform Aβ42 in CSF, an event that precedes the appearance of clinical symptoms. The onset of neurodegenerative symptoms is accompanied by increasing levels of tau and other biomarkers of neurodegeneration in CSF. As such, the temporal analysis of Aβ isoforms and tau in CSF can be used to diagnose and stage Alzheimer's disease. (See, e.g., Jack, et al., Lancet Neurol. 9:119-128, 2010, Blennow, et al., Nat. Rev. Neurol., 6:13-144, (2010); each of which is incorporated herein by reference).

Non-limiting examples of biomarkers for Alzheimer's Disease include adipocyte complement-related protein of 30 kDa (Acrp30), agouti-related protein (agouti-related transcript (AgRP/ART), amyloid beta, angiogenin (ANG), AXL, basic fibroblast growth factor (bFGF), bone morphogenetic protein 4 (BMP-4), bone morphogenetic protein 6 (BMP-6), brain-derived neurotrophic factor (BDNF), ciliary neurotrophic factor (CNTF), Eotaxin-2, epidermal growth factor (EGF), epidermal growth factor receptor (EGF-R), FAS, fibroblast growth factor 4 (FGF-4), fibroblast growth factor 6 (FGF-6), granulocyte-colony stimulating factor (GCSF), insulin-like growth factor binding protein 1 (IGFBP-1), insulin-like growth factor binding protein 2 (IGFBP-2), insulin-like growth factor binding protein 4 (IGFBP-4), intercellular adhesion molecule 1 (ICAM-1), interferon-gamma (IFN-g), interleukin 8 (IL-8), interleukin 1 receptor antagonist (IL-1ra), interleukin 3 (IL-3), interleukin-6 receptor (IL-6 R), LEPTIN(OB), macrophage inflammatory protein-(1d MIP-1d), macrophage inflammatory protein-1beta (MIP-1b), macrophage stimulating protein alpha (MSP-a), monocyte chemoattractant protein 1 (MCP-1), nerve growth factor (NGF), neurotrophin 3 (NT-3), neutrophil-activating peptide 2 (NAP-2), phospho-tau protein, platelet-derived growth factor BB (PDGF-BB), pulmonary and activation-regulated chemokine (PARC), RANTES, soluble tumor necrosis factor receptor II (sTNF RII), stem cell factor (SCF), tau protein, thrombopoietin (TPO), thymus and activation-regulated chemokine (TARC), tissue inhibitor of metalloproteinase 1 (TIMP-1), tissue inhibitor of metalloproteinase 2 (TIMP-2), transforming growth factor, beta 3 (TGF-b3), transthyretin, tumor necrosis factor beta (TNF-b), tumor necrosis factor-related apoptosis-inducing ligand receptor 3 (TRAIL R3), urokinase-type plasminogen activator receptor (uPAR), or vascular endothelial growth factor B (VEGF-B).

Non-limiting examples of pathological conditions or biomarkers associated with Alzheimer's disease include increased levels of one or more of total tau, phosphorylated-tau, or nerve growth factor, and decreased levels of Aβ42. The relative levels of tau and Aβ42 in CSF can be used to diagnose and monitor the progression of Alzheimer's disease. For example, in an embodiment, a decrease in Aβ42 by as much as about 50% in CSF as compared with healthy control levels, presumably secondary to the increased oligomer accumulation of Aβ isoforms in plaques and/or to reduced production in the central nervous system, is indicative of conditions associated with Alzheimer's disease. In an embodiment, a detected increase in both total tau and a phosphorylated form of tau in CSF is indicative of conditions associated with Alzheimer's disease. Phosphorylated tau in particular has reasonable diagnostic specificity for Alzheimer's disease and is consistently elevated in CSF in subjects with Alzheimer's disease as compared with other types of dementia (e.g., frontotemporal dementia, Lewy body dementia, and vascular dementia). Optimal diagnostic accuracy can be obtained when the levels of tau and Aβ42 in CSF are used in combination. In an embodiment, a detected elevated level of total-tau in CSF is indicative of a disease state associated with Alzheimer's disease or mild cognitive impairment compared with cognitively normal controls. The typical pattern of Aβ42 and tau levels in CSF of a subject with Alzheimer's disease is a measured decrease in Aβ42 (i.e., <500 ng/L) and a measured increase in total tau (>350 ng/L) and/or phosphorylated tau (>85 ng/L). The levels of total tau can be approximately 300% higher in CSF of a subject with Alzheimer's disease relative to normal subjects. In an embodiment, the monitoring both total tau and Aβ42 levels in CSF yields a sensitivity of 81% to 94% and a specificity of 79% to 95% for diagnosing Alzheimer's disease in a subject. (See, e.g., Verbeek & Olde Rikkert, Clin. Chem. 54:1589-1591, (2008); which is incorporated herein by reference).

In an embodiment, the implantable device 102 determines a progression of Alzheimer's disease by monitoring CSF levels of Aβ42, tau and phosphorylated tau. For example, increasing levels of phosphorylated tau and decreasing levels of Aβ42 over time are correlated with decreased memory and decreased hippocampal volume in subjects with mild cognitive impairment. (See e.g., de Leon, et al., Neurobiol Aging 27:394-401, (2006); which is incorporated herein by reference). In an embodiment, an increase in a measured level of one or more of NGF, total tau protein, or tau-phosphorylated protein; and a decrease in a measured level of one or more of Aβ42 or transthyretin can be indicative of an Alzheimer's disease state.

The levels of tau in CSF have been shown to increase over time whereas the levels of Aβ42 appear to remain unchanged (Bouwman et al., Clin Chem. 52:1604-1606, (2006); which is incorporated herein by reference). In an embodiment, conversion from mild cognitive impairment to Alzheimer's dementia and the rate of cognitive decline is determined (90% sensitivity and 100% specificity) based on the measurement of total-tau alone or in combination with phosphorylated-tau and Aβ42. In an embodiment, the implantable device 102 monitors disease progression by monitoring changes in the levels of tau and phosphorylated tau in the CSF relative to the levels of Aβ42. (See, e.g., Andreasen & Blennow, Clin. Neurol. Neurosurg., 107:165-173, (2005); Mistur, et al., J. Clin. Neurol. 5:153-166, (2009); Hampel, et al., Exp. Gerontol., 45:30-40, (2010); each of which is incorporated herein by reference).

Measurements of Aβ42, total tau, and phosphorylated tau can be combined with measurement of visinin-like protein 1 (VLP-1) for improved diagnostic performance, particularly in subjects with the APOE e4/E4 genotype predisposed to development of Alzheimer's disease. (See, e.g., Lee et al., Clin. Chem. 54:1617-1623, (2008); which is incorporated herein by reference). A diagnosis of Alzheimer's disease can also be made based on a relatively low level of Aβ42 in CSF, higher CSF/serum albumin ratio and higher levels of neurofilament protein light (NFL) in CSF.

In an embodiment, the implantable device 102 is operable to differentiate between a sporadic Alzheimer's disease state and familial Alzheimer's disease state by measuring relative levels of various amyloid β (Aβ) isoforms as compared with filtering information 234 associate with healthy subjects. For example, relatively low levels of Aβ1-42 and high levels of Aβ1-16 distinguish sporadic Alzheimer's disease and familial Alzheimer's disease from healthy controls, while very low levels of isoforms Aβ1-37, Aβ1-38, and Aβ1-39 distinguish familial Alzheimer's disease from sporadic Alzheimer's disease. (See, e.g., Portelius, et al., Mol. Neurodegener. 5:2, (2010); which is incorporated herein by reference).

Further non-limiting examples of biomarkers of Alzheimer's disease include albumin, amyloid Aβ A4 protein, angiotensinogen, apolipoprotein AI, apolipoprotein AII, apolipoprotein E, β-site APP-cleaving enzyme 1 (BACE1), amyloid precursor protein isoforms, truncated amyloid-β isoforms, amyloid-β oligomers, endogenous amyloid-β autoantibodies, 24S-hydroxycholesterol, C3a, C4a, cystatin C, immunoglobulin heavy chain, leucine-rich repeat-containing protein 4B, N-acetyllactosamine, neuronal pentraxin-1, prostaglandin-H2, retinol binding protein, thioredoxin, VGF, α-1-antitrypsin, α-1β glycoprotein, α-2HS glycoprotein, β fibrinogen, β-2-microglobulin, visinin-like protein1 (VLP-1), neurofilament, RAB3A, synaptotagmin, growth-associated protein (GAP-43), synaptosomal-associated protein 25, neurogranin, or F2-isoprostanes. In an embodiment, increasing CSF levels of isoprostane, a biomarker of oxidative stress associated with neurodegeneration, are correlated with associated with cognitive decline from MCI to Aβ. (See, e.g., Blennow, et al., Nat. Rev. Neurol., 6:131-144, 2010; Cedazo-Minguez & Winblad, Exp. Gerontol. 45:5-14, (2010); each of which is incorporated herein by reference).

In an embodiment, the system 100 includes an implantable device 102 configured to used to differentiate between Alzheimer's disease and other forms of dementia by monitoring Aβ42, total tau, or phosphorylated tau, as well as other biomarkers in CSF. For example, in an embodiment, detected increases in levels of total tau and phosphorylated tau and detected decreases in levels of are indicative of an Alzheimer's disease state. Levels of total tau and phosphorylated tau are moderately to strongly increased while the levels of Aβ42 are strongly decreased in CSF of subjects with Alzheimer's relative to normal age-matched filtering information.

In an embodiment, a detected increase in total tau in CSF while the detected levels of phosphorylated tau remain comparable to normal aged match filtering information (e.g., reference normal aged match control information, etc.) is indicative of a Creutzfeldt-Jakob disease state. In an embodiment, an elevated level of 14-3-3 protein is in CSF is indicative of a Creutzfeldt-Jakob disease state. In an embodiment, normal to slightly decreased CSF levels of Aβ42, normal to slightly increased CSF levels of total tau levels, and normal CSF levels of phosphorylated tau are indicative of a vascular dementia disease state.

In an embodiment, the system 100 includes an implantable device 102 configured to differentiate between Alzheimer's and other forms of dementia. For example, in an embodiment, the implantable device 102 compares CSF levels of one or more brain-derived metabolites to filtering information 234 and differentiate between Alzheimer's and other forms of dementia based on the comparison. For example, in an embodiment, relative levels of homovanillic acid (a metabolite of dopamine) and 5-hydroxyindoleacetic acid (a metabolite of serotonin) are used to differentiate between Alzheimer's disease and frontotemporal dementia (see, e.g., Engleborghs, et al., J. Neurol. Neurosurg. Psychiatry, 75:1080, (2004); which is incorporated herein by reference). Frontotemporal dementia refers to a group of neurodegenerative diseases that are commonly misdiagnosed as Alzheimer's disease. Unlike Alzheimer's disease, frontotemporal dementia is characterized by early age of onset as well as early changes in personality such as impulsive behavioral patterns. The ratio of HVA:5-HIAA ratio has been linked to aggressive and impulsive and antisocial behavior (see, e.g, Moore, et al., Aggressive Behavior, 28:299-316, (2002); which is incorporated herein by reference). In an embodiment, a ratio of HVA:5-HIAA is correlated with frontotemporal dementia but not with Alzheimer's disease.

In an embodiment, the system 100 includes an implantable device 102 configured to detect a formation or presence of a pathological condition associated with Parkinson's disease. For example, in an embodiment, the system 100 includes an implantable device 102 including at least one sensor component 204 operable to detect one or more markers for Parkinson's disease. Parkinsonian disorders represent a large group of neurodegenerative diseases, which are increasingly common with advancing age, with Parkinson's disease being the most widespread with a prevalence approaching 1% in subjects aged 65 years and older. Other less common atypical Parkinsonian disorders include multiple system atrophy, corticobasal degeneration, and dementia with Lew bodies. Parkinsonian disorders are characterized by tremor, rigidity, and bradykinesia along with pyramidal features, dysautonomic and cerebellar features, supranuclear gaze palsy, speech, cognitive and balance dysfunctions. While the various Parkinsonian disorders have similar early symptoms, progression and general outcomes vary with the atypical Parkinsonian disorders tending towards a faster progression rate and premature death. (See, e.g., Constantinescu, et al., Parkinsonism Relat. Disord., 15:205-212, 2009; which is incorporated herein by reference).

In an embodiment, the system 100 includes an implantable device 102 that differentiates between various Parkinsonian disorders by monitoring at least one of neurofilament light chain, phosphorylated neurofilament heavy chain, tau protein, Aβ42, glial fibrillary acidic protein, S-100β, neuron specific enolase, or myelin basic protein in CSF of a biological subject. For example, in an embodiment, the implantable device 102 includes one or more sensors 206 that detect CSF levels of neurofilament light chain and phosphorylated neurofilament. In an embodiment, elevated CSF levels of neurofilament light chain and phosphorylated neurofilament heavy chain are indicative of multiple system atrophy and progressive supranuclear palsy, but not of Parkinson's disease. In contrast, glial fibrillary acidic protein and neuron specific enolase remain relatively unchanged in CSF of subjects with Parkinson's disease and multiple system atrophy as compared with normal controls. In an embodiment, detected normal CSF levels of Aβ42 and tau protein are indicative of a Parkinson's disease state, whereas a detected decrease in Aβ42 levels and a detected increase in tau protein levels are indicative of Alzheimer's disease.

In an embodiment, neurofilament (heavy and/or light chains) and tau can be used to differentiate between multiple system atrophy (high levels of both neurofilament and tau), progressive supranuclear palsy (high levels of neurofilament, normal tau), and Parkinson's disease (normal levels of both neurofilament and tau). (See, e.g., Constantinescu, et al., Parkinsonism Relat. Disord., 15:205-212, (2009); Mollenhauer & Trankwalder, Mov. Disord., 24:1411-1426, (2009); each of which is incorporated herein by reference).

In an embodiment, a multiplex approach (e.g., multi-analyte profile, multi-targeted moiety, multiplex array. etc.) is used to distinguish between Alzheimer's disease and Parkinson's disease using tau, Aβ42, brain-derived neurotrophic factor (BDNF), IL-8, vitamin D binding protein (VDBP), apolipoprotein AII, and apolipoprotein E. Tau is increased and Aβ42 is decreased in subjects with Alzheimer's disease relative to subjects with Parkinson's disease or normal controls. VDBP and IL-8 are increased while BDNF, apoAII and apoE are decreased in both neurodegenerative diseases relative to controls. (See, e.g., Zhang, et al., Am. J. Clin. Path., 129:526-529, (2008); which is incorporated herein by reference).

Further non-limiting examples of pathological conditions or biomarkers associated with Parkinson's disease include increased CSF levels of 8-hydroxy-2′-deoxyguanosine and 8-hydroxyguanosine (predominant biomarker of oxidative DNA damage), or TNF-alpha (inflammation); and decreased CSF levels of beta-phenylethylamine, gamma-aminobutyric acid (GABA), or Aβ42 (in subjects with dementia associated with Parkinson's).

In an embodiment, a detected decrease, as compare to normal controls, of GABA CSF levels can be indicative of a Parkinson's disease. (See, e.g., Kuroda, et al., J. Neurol. Neurosurg. Psychiatry, 45:257-260, (1982); which is incorporated herein by reference). Further non-limiting examples of CSF biomarkers for Parkinson's disease and other movement disorders can be found in Mollenhauer & Trenkwalder, Mov. Disord., 24:1411-1426, (2009); which is incorporated herein by reference).

In an embodiment, the system 100 includes an implantable device 102 configured to detect a formation or presence of a pathological condition associated with Creutzfeldt-Jakob disease (CJD). For example, in an embodiment, the system 100 includes an implantable device 102 configured to detect one or more markers for CJD. CJD is a rare and fatal progressive neurodegenerative disease. It is the most common type of transmissible spongiform encephalopathy. CJD is a prion disease in which endogenous cellular prion protein is converted into a protease resistant, misfolded isoform, which accumulates in neural tissue. The disease is characterized by rapidly progressive dementia, leading to memory loss, personality changes, and hallucinations as well as physical impairments including speech impairments, jerky movements, balance and coordination dysfunction, changes in gait, rigid posture, and seizures.

In an embodiment, the implantable device 102 is configured to detect a pathological condition or biomarkers in CSF that is associated with a CJD state. Non-limiting examples of pathological conditions or biomarkers for CJD include increased levels of one or more of 14-3-3 protein, total tau (tTau), neuron specific enolase (NSE), S100β, nerve growth factor (NGF), cystatin C, transferring, ubiquitin, apolipoprotein J, or IL-8; and decreased levels of one or more of amyloid β42, fibroblast growth factor 2 (FGF2), or tumor growth factor beta 2 (TGFβ2). In an embodiment, the implantable device 102 is configured to monitor for 14-3-3 protein ins CSF. In an embodiment, detection of 14-3-3 along with an appropriate clinical profile can be indicative of sporadic CJD, the most common and aggressive form of CJD. (See, e.g., Van Everbroeck, et al., Clin. Neurol. Neurosurg., 107:355-360 (2005); which is incorporated herein by reference). The analysis of 14-3-3 protein can be combined with other biomarkers to aide in diagnosis of Creutzfeld-Jakob disease. For example, CSF levels of tau above 500 pg/ml, S100β above 0.5 ng/ml, and NSE above 20 ng/ml are positively correlated with histologically confirmed CJD. (See, e.g., Green, et al., J. Neurol. Neurosurg. Psychiatry, 70:744-748, (2001); which is incorporated herein by reference).

In an embodiment, higher levels of brain-derived proteins in CSF are associated with shorter survival. For example, a median tau protein level in CSF of approximately 6400 pg/ml or more predicts a disease duration of 5 months or less while a median tau protein level in CSF of approximately 4400 pg/ml or less predicts a disease duration of longer than 5 months. (See, e.g., Sanchez-Juan, et al., J. Neurol. 254:901-906, (2007); which is incorporated herein by reference). The ratio of total tau protein to 181 phosphorylated tau protein (Tau/P-Tau ratio) and total tau are also efficient biomarkers (see, e.g., Skinningsrud, et al., Cerebrospinal Fluid Res., 5:14, (2008); which is incorporated herein by reference).

Another example of a biomarker for CJD is heart type fatty acid binding protein (H-FABP). H-FABP is significantly elevated in CSF of subjects with CJD relative to normal controls or subjects with other neurodegenerative diseases. For example, the level of H-FABP in CSF of CJD subjects ranges from about 3,000 to 43,000 pg/ml while H-FABP in dementia Alzheimer's disease subjects ranges from about 2,000 to 5,700 pg/ml. Levels of H-FABP in CSF above 3,500 pg/ml are positively correlated with CJD. In an embodiment, a detected elevated CSF level of H-FABP in combination with elevated 14-3-3 protein or elevated tau is indicative of a CJD state. (See, e.g., Matsui, et al., Cell. Mol. Neurobiol., on-line publication May 25, (2010); which is incorporated herein by reference). In an embodiment, a reduction in the level of normal prion protein relative to normal controls in CSF is indication of CJD state as well as other neurodegenerative diseases. (See, e.g., Meyne, et al., J. Alzheimer's Dis., 17:863-873, (2009); which is incorporated herein by reference).

Further non-limiting examples of biomarkers for CJD include 14-3-3 protein, amyloid-β (Aβ42), Apolipoprotein J, cystatin C, FGF-2, heart fatty, nerve growth factor (NGF), neuron specific enolase (NSE), pro- and anti-inflammatory cytokines (cytokine IL-8, TGF-β2), S100b protein, transferrin, t-tau, and ubiquitin. In an embodiment, an increase in a measured level of one or more of 14-3-3 proteins, apolipoprotein J, cystatin C, heart fatty, IL-8, nerve growth factor (NGF), neuron specific enolase (NSE), S100b, transferrin, t-tau, and ubiquitin and a decrease in a measured level of one or more of Aβ42, FGF-2, TGF-beta 2 can be indicative of a diagnosis of CJD.

In an embodiment, the system 100 includes an implantable device 102 configured to detect a formation or presence of a pathological condition associated with multiple sclerosis. For example, the implantable device 102 is configured to detect one or more markers associated with multiple sclerosis. Multiple sclerosis is chronic autoimmune condition in which a subject's immune system attacks the central nervous system leading to demyelination of neurons and progressive neurodegeneration of the central nervous system. There are several subtypes or patterns of disease progression: relapsing-remitting, secondary progressive, primary progressive and progressive-relapsing. The relapsing-remitting subtype is characterized by unpredictable relapses (attacks) of disease symptoms followed by months to years of no new signs of disease. Secondary progressive disease describes those subjects whose relapsing-remitting disease has progressed to primary progressive disease with few if any remissions. Primary progressive disease is characterized by steady decline in neurological function with no remissions but few if any attacks. Progressive relapsing disease is characterized by steady decline superimposed by periodic attacks.

In an embodiment, the implantable device 102 monitors disease progression and severity of multiple sclerosis by monitoring changes in the relative concentrations of one or more biomarkers in CSF of a subject with multiple sclerosis. Non-limiting examples of pathological conditions or biomarkers in CSF for multiple sclerosis include increased levels of oligoclonal immunoglobulin G (IgG) bands, myelin basic protein (MBP), IFN-gamma, TNF, neurofilament light chain (NFL), glial fibrillary acid protein (GFAP), soluble triggering receptor expressed on myeloid cells 2 (sTREM-2), sorbitol, soluble E-selectin, soluble CD30, soluble intercellular adhesion molecule-1, soluble vascular cell adhesion molecule-1,24S-hydroxycholesterol, nitrous oxide metabolites, neural cell adhesion molecule, tau, actin, tubulin, 14-3-3 protein, fructose, lipid-specific immunoglobulin M, CD4(+)TNFalpha(+)IL-2(−)T cells, kappa free light chains, autoantibodies to oligodendroglial molecules transketolase (TK), autoantibodies to cyclic nucleotide phosphodiesterase type I (CNPase I), autoantibodies to collapsin response mediator protein 2, autoantibodies to tubulin beta4, DJ-1, arachidonoylethanolamine (anandamide, AEA), NSE, Hex A, IL-17, IL-6, chromogranin A, sHLA-G1/HLA-G5, or HLA-G5; and decreased levels of lactate, angiotensin II, chromogranin B, or secretogranin II. (See, e.g., Awad, et al., J. Neuroimmunol., 219:1-7, (2009); which is incorporated herein by reference).

In an embodiment, the implantable device 102 monitors albumin and immunoglobulins in CSF to determine a disease state associated with multiple sclerosis. For example, in an embodiment, detected multiple bands of immunoglobulins (oligoclonal bands of IgG and less commonly IgM) is indicative of a disease state associated with multiple sclerosis. IgG and albumin measured in CSF of normal subjects are derived from the serum, whereas increased IgG in CSF of subjects with active multiple sclerosis reflects increased production of IgG in the central nervous system.

In an embodiment, the implantable device 102 detects CSF levels of immunoglobulins and albumin, and determines CSF index. In an embodiment, A CSF index numerically expresses the ratio of IgG to albumin in CSF, to the ratio of IgG to albumin in the serum. In an embodiment, a CSF index above 0.85 is indicative of local CNS synthesis of IgG and is correlated with a diagnosis of multiple sclerosis, as well as neurosyphilis, subacute sclerosing panencephalitis, chronic CNS infections and CNS lupus erythematosus. (See, e.g., Hische & van der Helm, Clin. Chem., 33:113-114, (1987); Freedman, Multiple Sclerosis, 10:S31-S35, (2004); each of which is incorporated herein by reference).

In an embodiment, detected oligoclonal IgG production in CSF, but not in the serum, is a diagnostic result indicative of multiple sclerosis. In addition, levels of myelin basic protein (MBP) can be measured in CSF and are indicative of breakdown of the myelin sheath that surrounds and protects neurons. The levels of MBP are differentially increased in subjects with multiple sclerosis. For example, the levels of MBP are higher in subjects with polysymptomatic exacerbations relative to levels in subjects with monosymptomatic exacerbations; are correlated with the number of brain lesions, the severity of relapse, and the production of another biomarker of multiple sclerosis, immunoglobulin M; and are decreased in response to treatment with steroidal anti-inflammatory agents (see, e.g., Lamers, et al., Brain Res. Bull. 61:261-264, (2003); which is incorporated herein by reference). In an embodiment, the implantable device 102 determines the severity of disease, as well as treatment response, by monitoring of MBP in CSF.

In an embodiment, the implantable device 102 detects a condition associated with cognitive decline during relapse. For example, in an embodiment, the implantable device 102 monitors levels of somatostatin, a peptide hormone. In an embodiment, a decrease in a CSF level of somatostatin during the periods of relapse is indicative of cognitive decline during relapse. (See, e.g., Roca, Biological Psychiatry 46:551-556, (1999); which is incorporated herein by reference). Both neurofilament light chain (NFL) and glial fibrillary acidic protein (GFAP) are increased in subjects with multiple sclerosis relative to normal controls. NFL and GFAP are intermediate filament proteins associated with neurons and glial cells, respectively and their presence in CSF is indicative of the loss of neuron and glial cell integrity. During relapse, the levels of NFL increase in CSF while the levels of GFAP remain the same (See, e.g., Norgen, et al., Neurology 63:1586-1590, 2004; which is incorporated herein by reference). Oligoclonal bands of immunoglobulins are also increased in CSF of subjects with multiple sclerosis but are not altered during disease progression (See, e.g., Koch, et al., Eur J Neurol. 14:797-800 (2007); which is incorporated herein by reference). In an embodiment, the implantable device 102 is configured to assess the onset of relapse or remission by monitoring changes in the relative levels of somatostatin, NFL, GFAP, and oligoclonal bands over time.

In an embodiment, the system 100 includes an implantable device 102 configured to detect a formation or presence of a pathological condition associated with amyotrophic lateral sclerosis (ALS). For example, in an embodiment, the system 100 includes an implantable device 102 that detects one or more markers associated with ALS. ALS is a progressive neurodegenerative disease of unknown etiology that primarily targets the upper and lower motor neurons in the spinal cord, brainstem, and motor cortex, leading to increasing muscle weakness and muscle atrophy, culminating in respiratory failure. There is some evidence to suggest that immunological factors can be involved in the pathogenic mechanism of the disease. Activated peripheral blood T lymphocytes and elevated levels of tumor necrosis factor-α (TNF-α) and interleukin (IL)-6 have been detected in CSF of some subjects with ALS.

Non-limiting examples of biomarkers of ALS include neurofilament light chain protein, cystatin C, 4.8 kDa VGF peptide, 6.7 kDa cationic protein species, erythropoietin, growth hormone, insulin-like growth factor, insulin, transthyretin, 3-nitrotyrosin, neuroendocrine protein 7B2, or S100β. See, e.g., Ekegren, et al., J. Mass Spectrom., 43:559-571, (2008); which is incorporated herein by reference). In addition, the levels of various cytokines and growth factors are altered in CSF of subjects with ALS. For example, in an embodiment, the cytokines and growth factors VEGF, G-CSF, IFN-γ, CCL2, CCL4, CCL5, CCL11, CXCL8, CXCL10, TNF-α, IL1β, IL-7, IL-12, and IL-17 levels are higher in CSF of subjects with ALS relative to levels in subjects with other non-inflammatory neurological diseases or in normal subjects. (See, e.g., Tateishi, et al., J. Neuroimmunol., 122:76-81, (2010); which is incorporated herein by reference). Additional biomarkers for ALS include increased levels of 7B2CT, glutamate, CCL2/VEGF ratio (more specific to ALS than to Parkinson's and spinocerebellar ataxia), and decreased levels of cystatin C, angiotensin II, and cytochrome C.

In an embodiment, the system 100 includes an implantable device 102 configured to detect a formation or presence of a pathological condition associated with traumatic brain injury. For example, in an embodiment, the system 100 includes an implantable device 102 configured to detect one or more pathological conditions or biomarkers associated with neuron death or astrocyte death. Non-limiting examples of pathological conditions or biomarkers for traumatic brain injury include increased neurofilament light chain (cerebrovascular accidents, subarachnoid hemorrhage and severe traumatic brain injury), neuron-specific enolase, S100β, spectrin, C-reactive protein, or myelin-basic protein. In an embodiment, a detected level of these biomarkers in CSF can be indicative of neuron and astrocyte death associated with the brain injury.

In the United States, there are more than 1 million traumatic brain injury cases annually, resulting in more than 230,000 hospitalizations, 50,000 deaths, and 80,000 subjects with disabilities. In an embodiment, confirmation of the injury mechanism using biomarkers can aide in identifying candidate drug therapy targets. For example, traumatic brain injury produces breakdown products of aThspectrin that are released into the CSF and are potential biomarkers for brain injury. The αII-spectrin breakdown products (SBDP) are generated in response to activation of the proteolytic enzyme calpain, a protease associated with necrotic neurodegeneration. Specifically, SBDP 150 and SBDP 145 are elevated in CSF three to four fold within 12 hours following head injury and remain significantly elevated up to 72 hours post injury relative to normal controls. (See e.g., Brophy et al., J. Neurotrauma, 26:471-479, (2009); which is incorporated herein by reference).

Traumatic brain injury can be difficult to diagnose in some subject populations such as, for example, in non-verbal infants who after an injury exhibit non-specific symptoms (e.g., high temperature, vomiting without diarrhea, seizures, and/or lethargy or fussiness). Analysis of CSF for biomarkers of traumatic brain injury in this population can include neuron-specific enolase (cutoff value of 11.77 mg/ml), S100B (cutoff value of 0.017 mg/ml), and myelin-basic protein (cutoff value of 0.30 mg/ml). (See, e.g., Berger, et al., Pediatrics, 117:325-332, (2006); which is incorporated herein by reference).

In an embodiment, increased levels of IL-6 in CSF (ranging from 400-900 pg/ml) as compared with levels in normal controls (0-75 pg/ml) can be indicative of an inflammatory response following head injury. (See, e.g., Is, et al., J. Clin. Neurosci., 14:1163-1171, 2007; which is incorporated herein by reference). Nerve growth factor and IL1β are also significantly increased in CSF of subjects with severe head injury. In an embodiment, increased levels of nerve growth factor and IL-6 in CSF at 48 hours following head injury are associated with favorable neurologic outcome whereas increased levels of IL1β in CSF at 48 hours are significantly associated with poor neurologic outcome as measured by Glasgow Outcome Scores. (See, e.g., Chiaretti, et al., Eur. J. Paediatr. Neurol., 12:195-204, (2007); which is incorporated herein by reference).

In an embodiment, the system 100 includes an implantable device 102 configured to detect a formation or presence of a pathological condition associated with ischemia. For example, in an embodiment, the system 100 includes an implantable device 102 configured to detect one or more markers associated with ischemia. Ischemia is a restriction of blood flow to body tissues that can lead to hypoxia, tissue damage, and ultimately death. Ischemia can be caused by a number of conditions including, among others, surgically induced circulation arrest, atherosclerosis, septic shock, heart failure, tachycardia, stroke, and thromboembolism. When ischemia or prolonged hypoxia occurs in the brain, severe damage to the tissue can occur, leading to brain injury. Measurement of biomarkers in CSF can be used to assess prognosis following isolated and/or global cerebral ischemia. For example, stroke severity can be predicted by measuring S100β in CSF. The level of S100β increases as much as 10 fold in CSF in stroke victims and is proportional to the severity and volume of the stroke. (See, e.g., Petzold, et al., J. Stroke Cardiovasc. Dis., 17:196-203, (2008); which is incorporated herein by reference).

Non-limiting examples of pathological conditions or biomarkers indicative of central nervous system ischemia (surgically induced or otherwise) include increased CSF levels of 14-3-3β protein, 14-3-3ζ protein, a hypophosphorylated form of a neurofilament subunit (pNFH), the deubiquitinating enzyme UCH-L1, or calpain-derived proteolytic fragments of α-spectin. (See, e.g., Siman, et al., Brain Res., 1213:1-11, (2008); which is incorporated herein by reference).

Subjects undergoing repair of an aneurysm of the descending or thoracoabdominal aorta are at greater risk of damage to the central nervous system, (e.g., immediate or delayed paraplegia) following the surgical procedure. Analysis of CSF before and after the surgical procedure suggests that a 10-500 fold increase in glial fibrillary acidic protein (GFAP) is correlated with increased risk of neurological damage such as, for example, paralysis and stroke. Spinal symptoms are further noted in subjects who exhibit increased cerebrospinal levels of neurofilament light chain (10 fold increase) and S10013 (10 fold increase). (See, e.g., Anderson, et al., J. Cardiothorac. Vasc. Anesth., 17:598-603, (2003); Winnerkvist, et al., Eur. J. Cardiothorac. Surg., 31:637-642, (2007); each of which is incorporated herein by reference). Delayed paraplegia can be mitigated by administering immediate countermeasures, e.g., continued use of CSF drain.

In an embodiment, the system 100 includes an implantable device 102 configured to predict prognosis following cerebral ischemia by monitoring one or more pathological conditions or biomarkers in CSF. For example, detected increases in creatine kinase (>200 U/l), detected increases in one or more of neuron specific enolase (>33 ng/ml), lactate dehydrogenase (>82 U/l), or glutamate oxaloacetate (>62 U/l) in CSF of a subject who has experienced cerebral ischemia is indicative of poor prognosis defined as death or persistent vegetative state in anoxic-ischemic coma. (See, e.g., Zandbergen, et al., Intensive Care Med., 27:1661-1667, (2001); which is incorporated herein by reference).

In an embodiment, the system 100 includes an implantable device 102 that detects a formation or presence of a pathological condition associated with meningitis. For example, in an embodiment, the system 100 includes an implantable device 102 configured to detect one or more biomarkers associated with meningitis. Meningitis is a medical condition characterized by inflammation of the meninges, the protective membranes that cover the brain and spinal cord which can lead to long term neuropsychiatric deficits and death in the most severe cases. Meningitis can be caused by bacteria, viruses or other infectious microorganisms as well as cancer and certain types of medications. The analysis of CSF can be used to diagnose meningitis. Non-limiting examples of pathological conditions or biomarkers for meningitis include increased levels of total protein, white blood cells (specifically neutrophils), C-reactive protein, lactate, complement C3 and C5, CXCL8, CXCL1, CCL2, CCL3, CCL4, MMP-9, IL-16, uPA, or uPAR; and decreased levels of glucose relative to serum levels. For example, a detected increase in levels of neutrophils (>0), total protein (>2 g/liter), C-reactive protein (>100 mg/liter), lactate (>5 mmol/liter) in CSF of a subject combined with decreased CSF levels of glucose (<2 mmol/liter) of a subject are indicative of bacterial meningitis. (See, e.g., Holub, et al., Critical Care, 11:R41, (2007); which is incorporated herein by reference).

In an embodiment, the system 100 includes an implantable device 102 configured to differentiate between aseptic meningitis and bacterial meningitis, an important distinction when determining the appropriateness of antibiotic treatment (see, e.g., Negrini, et al., Pediatrics, 105:316-319, (2000); Holub, et al., Critical Care, 11:R41, (2007); each of which is incorporated herein by reference). In an embodiment, the system 100 includes an implantable device 102 that monitors at least one of white blood cell levels, protein levels, or glucose levels in CSF. In an embodiment, detected CSF levels of white cells are use to differentiate between aseptic meningitis and bacterial meningitis. For example, the number of white blood cells detected in CSF of subjects with bacterial meningitis is on average 7 times higher than the median number of cells detected in CSF of subjects with aseptic meningitis. In an embodiment, a detected increase in the CSF level of white cells can be indicative of subjects with bacterial meningitis.

In an embodiment, the system 100 includes an implantable device 102 configured to differentiate between aseptic meningitis and bacterial meningitis by monitoring CSF level of one or more biomarkers. For example subjects with bacterial meningitis can exhibit approximately three times the protein levels in CSF but approximately half the glucose levels relative to subjects with aseptic meningitis. In an embodiment, detected elevated levels of one or more of TNF-α, IL-1β, IL-6, IL-8, IL-10, or cortisol are indicative of bacterial meningitis. For example, detection of IL-6 in CSF at levels ranging from about 1000 to about 15,000 fold higher than normal levels is indicative of bacterial meningitis whereas levels of about 2 to about 20 fold higher than normal levels is more indicative of aseptic meningitis (see, e.g., Holub, et al., Critical Care, 11:R41, (2007); which is incorporated herein by reference). In an embodiment, the system 100 includes an implantable device 102 configured to differentiate between aseptic meningitis and bacterial meningitis by monitoring cell counts, protein levels, and glucose levels.

In an embodiment, during the course of bacterial meningitis, fluctuations in the levels of one or more components of the CSF can be indicative of progression or prognosis. For example, in an embodiment, CSF levels of neuron-specific enolase (NSE) are used as a potential indicator of progression and outcome in subjects with bacterial meningitis. In an embodiment, high CSF levels of NSE are indicative of bacterial meningitis relative to that of subjects with other infectious diseases of the central nervous system. (See, e.g., Inoue, et al., Acta. Paediatr Jpn. 36:485-488, (1994); which is incorporated herein by reference). In juvenile subjects with bacterial meningitis, an increase in NSE above 25 ng/ml in the CSF during the acute phase is positively correlated with increased incident of subdural effusion, the collection of pus beneath the outer lining of the brain. In those subjects in which NSE levels rise again during the course of disease, central nervous system complications can be more pronounced.

In an embodiment, the system 100 includes an implantable device 102 configured having one or more sensors for detecting the color of CSF. In an embodiment, the color of the CSF is monitored, in addition to the cellular and biomolecule composition, to aide in diagnosis and status of meningitis. For example, the color of CSF can be indicative of various pathologies associated with meningitis (see, e.g., Seehusen, et. al., Am. Fam. Physician 68:1103-1108, 2003; which is incorporated herein by reference). In an embodiment, detected yellow fluid is indicative of the presence of blood breakdown products, hyperbilirubinemia, and increased total protein greater than or equal to 1.5 grams per liter; orange fluid is indicative of the presence of blood breakdown products, and high carotenoid ingestion; pink fluid is indicative of the presence of blood breakdown products; green fluid is indicative of hyperbilirubinemia and purulent CSF; and brown fluid is indicative of meningeal melanomatosis.

A normal level of lactate in CSF is usually below 2.5 mmol/liter. Increased levels of lactate in CSF can be indicative of cerebral hypoxia, bacterial meningitis, or some inherited mitochondrial diseases. Other small molecule components include creatinine, glucose, iron, phosphorus, and urea. The normal level of glucose in CSF ranges from about 60-80% of the corresponding plasma levels. In an embodiment, a detected CSF:plasma ratio below about 0.6 can be indicative of bacterial infection or hypoxia. The normal level of total proteins in CSF range from about 0.2 to about 0.8 gm/liter, depending upon the age of the individual. By contrast, normal blood has a protein concentration of about 60 gm/liter.

In an embodiment, the system 100 includes an implantable device 102 configured to detect a formation or presence of a pathological condition associated with encephalitis. For example, in an embodiment, the system 100 includes an implantable device 102 configured to detect a progression of encephalitis or predict a disease outcome by monitoring levels of at least one of neuron-specific enolase (NSE), S100β, or myelin basic protein (MBP). Encephalitis is an acute inflammation of the brain, commonly caused by a viral infection or as a complication of bacterial meningitis, rabies or syphilis. Certain parasitic or protozoal infections such as toxoplasmosis, malaria or primary amoebic meningoencephalitis can also cause encephalitis in immune compromised subjects. The symptoms associated with encephalitis are similar to those associated with meningitis, e.g., fever, headache, photophobia, and less frequently, muscle stiffness. Like meningitis, the CSF in subjects with encephalitis can have elevated proteins levels and white blood cells, although not always. Unlike meningitis, the glucose levels in CSF remain at normal levels. As such, analysis of the CSF can be used to differentiate between encephalitis and meningitis. In an embodiment, the system 100 includes an implantable device 102 that detects encephalitis by monitoring for neuron-specific enolase (NSE), S10013, or myelin basic protein (MBP) in CSF (see, e.g., van Engelen, et al., Clin. Chem. 38:813-816, (1992); which is incorporated herein by reference). In an embodiment, the presence of one or more of NSE, S10013, or MBP at levels above a reference threshold is indicative of the onset of encephalitis due to herpes infection (which can be confirmed by using antibodies or DNA amplification to detect the herpes virus in CSF). In an embodiment, the system 100 includes an implantable device 102 that monitors of NSE, S100β, and MBP in CSF to assess treatment efficacy. For example, in an embodiment, normalization of NSE and S100β levels but continued high levels of MBP following the normal course of treatment can be indicative of continued demyelination and of a worse prognosis for the subject.

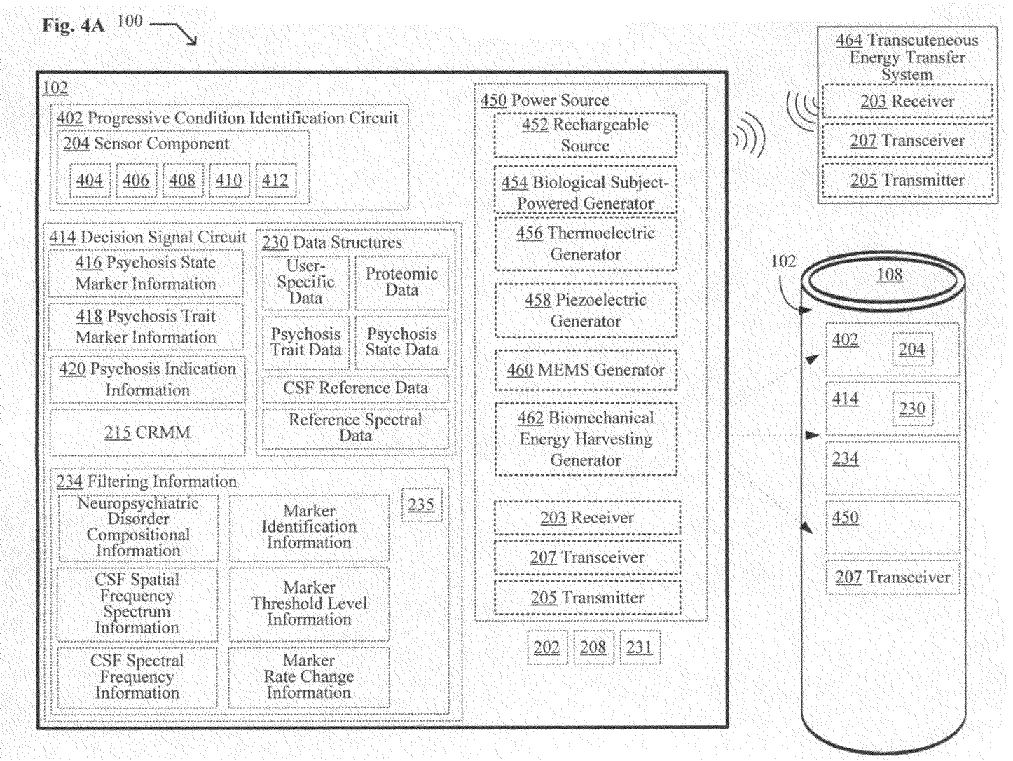

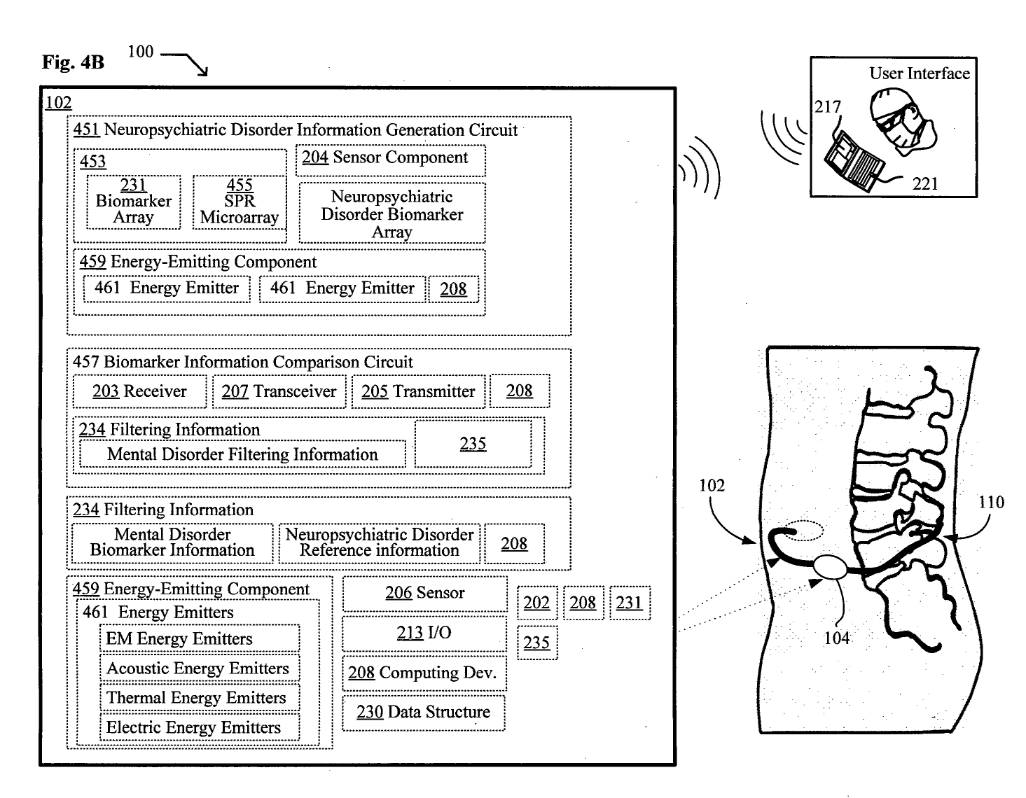

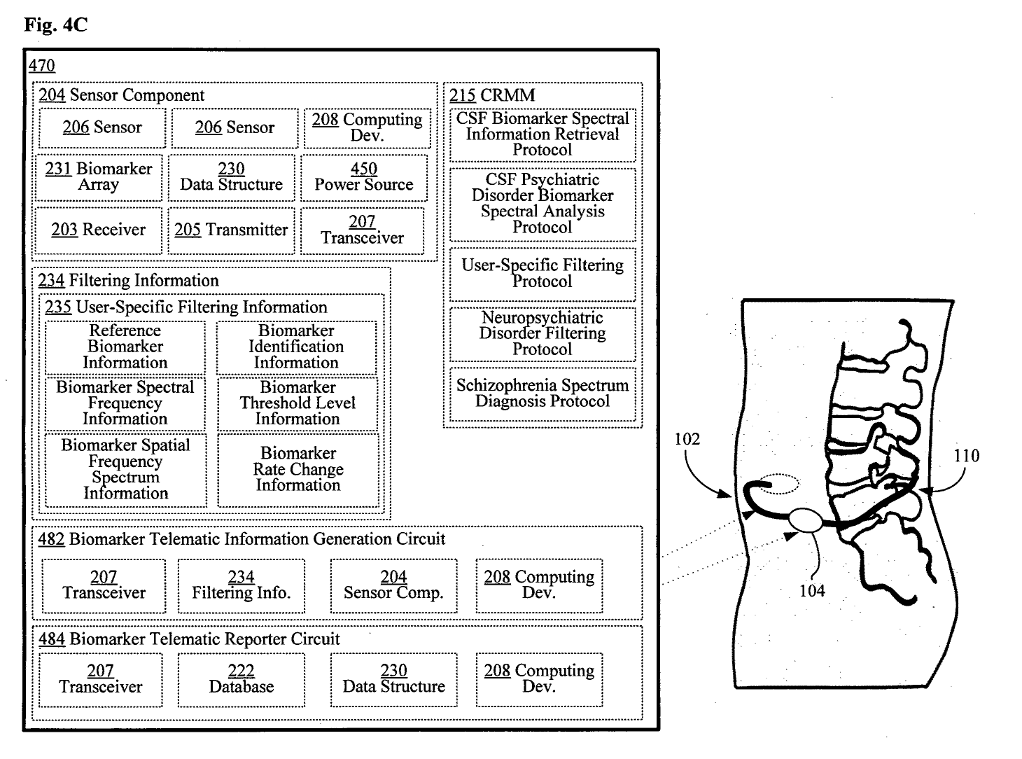

Referring to FIG. 4A, in an embodiment, the system 100 includes a progressive condition identification circuit 402 configured to obtain in vivo CSF information of CSF proximate a surface of an indwelling shunt. In an embodiment, the progressive condition identification circuit 402 includes at least one of a thermal detector, a photovoltaic detector, a photomultiplier detector, a charge-coupled device, a complementary metal-oxide-semiconductor device, a photodiode image sensor device, a Whispering Gallery Mode micro cavity device, a scintillation detector device, a photo-acoustic spectrometer, a thermo-acoustic spectrometer, or a photo-acoustic/thermo-acoustic tomographic spectrometer. In an embodiment, the progressive condition identification circuit 402 includes one or more ultrasonic transducers 404. In an embodiment, the progressive condition identification circuit 402 includes at least one of a time-integrating optical component, a linear time-integrating component, a nonlinear optical component, or a temporal auto correlating component.

In an embodiment, the progressive condition identification circuit 402 includes one or more one-, two-, or three-dimensional photodiode arrays 406. In an embodiment, the progressive condition identification circuit 402 includes at least one SPR sensor 408, wavelength-tunable SPR sensor 410. In an embodiment, the progressive condition identification circuit 402 includes at least one image-generating component 412 for generating an image of CSF applied to a molecule detection array.

In an embodiment, the progressive condition identification circuit 402 includes at least one image-generating component 412 for generating an n-dimensional expression profile vector of the CSF proximate the surface of an indwelling shunt. In an embodiment, the progressive condition identification circuit 402 includes at least one SPR microarray sensor, the at least one SPR microarray sensor having an array of micro-regions configured capture respective target molecules. In an embodiment, the progressive condition identification circuit 402 includes at least one SPR microarray sensor having a wavelength-tunable metal-coated grating. In an embodiment, the progressive condition identification circuit 402 includes at least one microarray sensor, biomedical array sensor, peptide array sensor, antibody array sensor, nucleic acid array, deoxyribonucleic acid array, ribonucleic acid array, protein microarray, or protein in situ array.

In an embodiment, the system 100 includes a decision signal circuit 414 configured to signal a decision whether to transmit a notification in response to one or more comparisons between filtering information 234 specific to the biological subject and obtained in vivo CSF information. In an embodiment, the decision signal circuit 414 includes one or more computer-readable memory media 215 having reference CSF information configured as a data structure 230. In an embodiment, the data structure 230 includes at least one of psychosis state marker information 416, psychosis trait marker information 418, or psychosis indication information 420. In an embodiment, the data structure 230 includes at least one of proteomic profile information, peptidomic profile information, or metabolic profile information. In an embodiment, the data structure 230 includes at least one of proteomic information indicative of a prodrome for a psychosis, peptidomic information indicative of a prodrome for a psychosis, or metabolic information indicative of a prodrome for a psychosis. In an embodiment, the decision signal circuit 414 includes one or more computer-readable memory media 215 having biological subject specific filtering information configured as a data structure 230, the biological subject specific filtering information including neuropsychiatric disorder compositional information.

In an embodiment, the system 100 includes circuitry configured to generate a first response based at least in part on the one or more comparisons between the filtering information 234 specific to the biological subject and the obtained in vivo CSF information of the biological subject. In an embodiment, the system 100 includes circuitry configured to selectively tune at least one of a wavelength distribution of an interrogation electromagnetic energy or a wavelength distribution of a detected electromagnetic energy. In an embodiment, the system 100 includes circuitry configured to decide whether to report obtained in vivo CSF information and when to report obtained in vivo CSF information. For example, in an embodiment, the system 100 includes a receiver 203 configured to receive request-to-report information. In an embodiment, the system 100 includes a receiver 203 that transcutaneously receives filtering information. In an embodiment, the system 100 includes a receiver 203 that obtains filtering information 234. In an embodiment, the system 100 includes a receiver 203 that wirelessly receives user-specific treatment filtering information. In an embodiment, the system 100 includes a transmitter 205 configured to wirelessly report obtained in vivo cerebrospinal fluid information. In an embodiment, the system 100 includes a transmitter 205 configured to wirelessly report one or more responses based at least in part on the one or more comparisons between the mental disorder filtering information and the obtained in vivo cerebrospinal fluid information of the biological subject. In an embodiment, the system 100 includes a transmitter 205 configured to transcutaneously report obtained in vivo cerebrospinal fluid information. In an embodiment, the system 100 includes a transmitter 205 configured to transcutaneously report one or more responses based at least in part on the one or more comparisons between the mental disorder filtering information and the obtained in vivo cerebrospinal fluid information of the biological subject.

In an embodiment, the system 100 includes a CSF marker detection circuit configured to obtain in vivo CSF information of CSF proximate a surface of an indwelling shunt. In an embodiment, the system 100 includes a decision signal circuit 414 configured to signal a decision whether to transcutaneously transmit a notification in response to one or more comparisons between user-specific filtering information 235 and obtained in vivo CSF information. In an embodiment, the system 100 includes circuitry configured to obtain in vivo CSF compositional information of CSF proximate a surface of an indwelling shunt. In an embodiment, the system 100 includes circuitry configured to transcutaneously transmit a notification in response to one or more comparisons between filtering information 234 associated with a biological subject having the indwelling shunt within and obtained in vivo CSF compositional information. In an embodiment, the system 100 includes a circuit configured to compare acquired in vivo CSF spectral information to user-specific filtering information 235 and to transcutaneously transmit a notification in response to comparing of the acquired in vivo CSF spectral information to the user filtering information.

In an embodiment, the system 100 includes, among other things, one or more power sources 450. In an embodiment, the implantable device 102 includes one or more power sources 450. In an embodiment, the power source 450 is electromagnetically, magnetically, acoustically, optically, inductively, electrically, or capacitively coupled to at least one of a biomarker detection circuit 202, biomarker identification circuit 232, a sensor component 204, a computing device 208, or the like. Non-limiting examples of power sources 450 examples include one or more button cells, chemical battery cells, a fuel cell, secondary cells, lithium ion cells, micro-electric patches, nickel metal hydride cells, silver-zinc cells, capacitors, super-capacitors, thin film secondary cells, ultra-capacitors, zinc-air cells, or the like. Further non-limiting examples of power sources 450 include one or more generators (e.g., electrical generators, thermo energy-to-electrical energy generators, mechanical-energy-to-electrical energy generators, micro-generators, nano-generators, or the like) such as, for example, thermoelectric generators, piezoelectric generators, electromechanical generators, biomechanical-energy harvesting generators, or the like. In an embodiment, the power source 450 includes at least one rechargeable power source 452. In an embodiment, the implantable device 102 carries the power source 450. In an embodiment, the implantable device 102 includes at least one of a battery, a capacitor, or a mechanical energy store (e.g., a spring, a flywheel, or the like).

In an embodiment, the power source 450 is configured to manage a duty cycle associated with detecting the at least one biomarker profile of CSF received within the one or more fluid-flow passageways 108. In an embodiment, the power source 450 is configured to manage a duty cycle associated with comparing the detected at least one biomarker profile of CSF received within the one or more fluid-flow passageways 108. In an embodiment, the implantable device 102 is configured to provide a voltage, via a power source 450 operably coupled to at least one of the biomarker detection circuit 202 or biomarker identification circuit 232. In an embodiment, the power source 450 is configured to wirelessly receive power from a remote power supply. In an embodiment, the implantable device 102 includes one or more power receivers configured to receive power from an in vivo or ex vivo power source. In an embodiment, the power source 450 is configured to wirelessly receive power via at least one of an electrical conductor or an electromagnetic waveguide. In an embodiment, the power source 450 includes one or more power receivers configured to receive power from an in vivo or ex vivo power source. In an embodiment, the in vivo power source includes at least one of a thermoelectric generator, a piezoelectric generator, a microelectromechanical systems generator, or a biomechanical-energy harvesting generator.

In an embodiment, the implantable device 102 includes one or more generators configured to harvest mechanical energy from, for example, acoustic waves, mechanical vibration, blood flow, or the like. For example, in an embodiment, the power source 450 includes at least one of a biological-subject (e.g., human)-powered generator 454, a thermoelectric generator 456, a piezoelectric generator 458, an electromechanical generator (e.g., a microelectromechanical systems (MEMS) generator 460, or the like), a biomechanical-energy harvesting generator 462, or the like.

In an embodiment, the biological-subject-powered generator 454 is configured to harvest thermal energy generated by the biological subject. In an embodiment, the biological-subject-powered generator 454 is configured to harvest energy generated by the biological subject using at least one of a thermoelectric generator 456, a piezoelectric generator 458, an electromechanical generator 460 (e.g., a microelectromechanical systems (MEMS) generator, or the like), a biomechanical-energy harvesting generator 462, or the like. For example, in an embodiment, the biological-subject-powered generator 454 includes one or more thermoelectric generators 456 configured to convert heat dissipated by the biological subject into electricity. In an embodiment, the biological-subject-powered generator 454 is configured to harvest energy generated by any physical motion or movement (e.g., walking,) by biological subject. For example, in an embodiment, the biological-subject-powered generator 454 is configured to harvest energy generated by the movement of a joint within the biological subject. In an embodiment, the biological-subject-powered generator 454 is configured to harvest energy generated by the movement of a fluid (e.g., biological fluid) within the biological subject.

In an embodiment, the system 100 includes, among other things, a transcutaneous energy transfer system 464. In an embodiment, the implantable device 102 includes a transcutaneous energy transfer system 464. For example, in an embodiment, the implantable device 102 includes one or more power receivers configured to receive power from at least one of an in vivo or an ex vivo power source. In an embodiment, the transcutaneous energy transfer system 464 is electromagnetically, magnetically, acoustically, optically, inductively, electrically, or capacitively coupled to at least one of the biomarker detection circuit 202, the biomarker identification circuit 232, progressive condition identification circuit 402, the decision signal circuit 414, the computing device 208, or the sensor component 104. In an embodiment, the transcutaneous energy transfer system 464 is configured to transfer power from at least one of an in vivo or an ex vivo power source to the implantable device 102. In an embodiment, the transcutaneous energy transfer system 464 is configured to transfer power to the implantable device 102 and to recharge a power source 450 within the implantable device 102.

In an embodiment, the transcutaneous energy transfer system 464 is electromagnetically, magnetically, acoustically, optically, inductively, electrically, or capacitively coupleable to an in vivo power supply. In an embodiment, the transcutaneous energy transfer system 464 includes at least one electromagnetically coupleable power supply, magnetically coupleable power supply, acoustically coupleable power supply, optically coupleable power supply, inductively coupleable power supply, electrically coupleable power supply, or capacitively coupleable power supply. In an embodiment, the energy transcutaneous transfer system is configured to wirelessly receive power from a remote power supply.

In an embodiment, the system 100 includes one or more receivers 203, transmitters 205, or transceivers 207. In an embodiment, the system 100 includes a data structure 230 having s at least one of mental disorder state marker information, mental disorder trait information, or heuristically determined mental disorder information stored thereon.

FIG. 4B show an implantable shunt system 100 in which one or more methodologies or technologies can be implemented such as, for example, monitoring for a formation or presence of a pathological condition associated with neuropsychiatric disorder. In an embodiment, the implantable shunt system 100 includes a neuropsychiatric disorder information generation circuit 451 configured to generate neuropsychiatric disorder biomarker information of CSF applied to an array (e.g., a biomarker array 231, etc). In an embodiment, the neuropsychiatric disorder information generation circuit 451 includes at least one component 453 configured to generate an n-dimensional expression profile vector of a portion of the CSF. In an embodiment, the neuropsychiatric disorder information generation circuit 451 includes at least one SPR microarray sensor 455. In an embodiment, the SPR microarray sensor 455 includes an array of micro-regions configured to capture target molecules. In an embodiment, the neuropsychiatric disorder information generation circuit 451 includes at least one of a biomedical array 231, a chemical compound array, a neuropsychiatric disorder marker microchip array, an antibody array, a deoxyribonucleic acid array, a peptide array, a neuropsychiatric disorder protein array, or a neuropsychiatric disorder protein in situ array.

In an embodiment, the neuropsychiatric disorder information generation circuit 451 includes at least one sensor component 204 electromagnetically coupled to a neuropsychiatric disorder biomarker array. In an embodiment, the neuropsychiatric disorder information generation circuit 451 includes one or more memory structures 222 that store generated neuropsychiatric disorder biomarker information. In an embodiment, the neuropsychiatric disorder information generation circuit 451 includes one or more memory structures 222 that store time and composition information associated with the generated neuropsychiatric disorder biomarker information.

In an embodiment, the implantable shunt system 100 includes a biomarker information comparison circuit 457 configured to generate a comparison between the generated neuropsychiatric disorder biomarker information and user-specific filtering information 235. In an embodiment, the biomarker information comparison circuit 457 includes one or more memory structures for storing paired and unpaired data associated with the generated neuropsychiatric disorder biomarker information. In an embodiment, the biomarker information comparison circuit 457 includes at least one transceiver 207 operably coupled to the array, the transceiver 207 configured to transmit information associated with the comparison between the generated neuropsychiatric disorder biomarker information and the user-specific filtering information 235. In an embodiment, the biomarker information comparison circuit 457 includes one or more memory structures 222 for storing comparison information associated with the generated neuropsychiatric disorder biomarker information and user-specific filtering information 235. In an embodiment, the biomarker information comparison circuit 457 includes at least one transceiver operably coupled to the array, the transceiver 207 configured to transmit information associated with the generated neuropsychiatric disorder biomarker information of the cerebrospinal fluid applied to the array. In an embodiment, the biomarker information comparison circuit 457 includes one or more memory structures 222 for storing time paired and unpaired data information associated with the generated associated with the generated comparison between the generated neuropsychiatric disorder biomarker information and user-specific filtering information 235. In an embodiment, the implantable shunt system 100 includes at least one computing device 208 operably coupled to at least one of the neuropsychiatric disorder information generation circuit 451 or the biomarker information comparison circuit 457, and configured to activate the neuropsychiatric disorder information generation circuit 451 or the biomarker information comparison circuit 457 based on target criteria.

In an embodiment, the biomarker information comparison circuit 457 includes a receiver 203 for acquiring filtering information 234. In an embodiment, the biomarker information comparison circuit 457 includes a transceiver 207 for transcutaneously requesting filtering information 234. In an embodiment, the biomarker information comparison circuit 457 includes a receiver 203 configured to receive a request to transmit at least one of filtering information 234, comparison information, or neuropsychiatric disorder biomarker information. In an embodiment, the biomarker information comparison circuit 457 includes a receiver 203 configured to receive at least one of a request to activate the biomarker information comparison circuit, a request to transmit comparison information, or a request to activate the biomarker information comparison circuit and to report comparison information. In an embodiment, the implantable device 102 includes a communication circuit configured to transcutaneously communicate comparison information associated with comparing the detected at least one biomarker profile of CSF received within the one or more fluid-flow passageways 108 to the filtering information 234.

In an embodiment, the implantable shunt system 100 includes an energy-emitting component 459 configured to emit one or more energy stimuli (e.g., one or more electromagnetic stimuli, electrical stimuli, acoustic stimuli, and thermal stimuli, or the like). For example, in an embodiment, the implantable device 102 includes an energy-emitting component 459 configured to deliver an interrogation stimulus to a sample proximate the implantable device 102, the interrogation stimulus at a dose sufficient to elicit a spectral response from the sample proximate the implantable device 102. In an embodiment, the spectral response is process to determine a CSF biomarker profile and to determine a disease state of a subject associated with the sample.

In an embodiment, the energy-emitting component 459 is configured to generate one or more continuous or pulsed energy waves, or combinations thereof. In an embodiment, the energy-emitting component 459 is configured to deliver an interrogation energy stimulus to one or more region proximate the implantable device 102. In an embodiment, the energy-emitting component 459 is configured to deliver an emitted energy to a biological specimen (e.g., tissue, biological fluid, target sample, CSF, or the like) proximate the implantable device 102.

Energy-emitting components 459 forming part of the implantable device 102 can take a variety of forms, configurations, and geometrical patterns including for example, but not limited to, a one-, two-, or three-dimensional array, a pattern comprising concentric geometrical shapes, a pattern comprising rectangles, squares, circles, triangles, polygons, any regular or irregular shapes, or the like, or any combination thereof. One or more of the energy-emitting components 459 can have a peak emission wavelength in the x-ray, ultraviolet, visible, infrared, near infrared, terahertz, microwave, or radio frequency spectrum. In an embodiment, at least one of the one or more energy-emitting components 459 is configured to deliver one or more charged particles. In an embodiment, the energy-emitting components 459 includes one or more energy emitters 461.

Non-limiting examples of energy emitters 461 include electromagnetic energy emitters, acoustic energy emitters, thermal energy emitters, or electrical energy emitters. Further non-limiting examples of energy emitters 461 include optical energy emitters and ultrasound energy emitters. Further non-limiting examples of energy emitters 461 include, electric circuits, electrical conductors, electrodes (e.g., nano- and micro-electrodes, patterned-electrodes, electrode arrays (e.g., multi-electrode arrays, micro-fabricated multi-electrode arrays, patterned-electrode arrays, or the like), electrocautery electrodes, or the like), cavity resonators, conducting traces, ceramic patterned electrodes, electro-mechanical components, lasers, quantum dots, laser diodes, light-emitting diodes (e.g., organic light-emitting diodes, polymer light-emitting diodes, polymer phosphorescent light-emitting diodes, microcavity light-emitting diodes, high-efficiency UV light-emitting diodes, or the like), arc flashlamps, incandescent emitters, transducers, heat sources, continuous wave bulbs, ultrasound emitting elements, ultrasonic transducers, thermal energy emitting elements, or the like. In an embodiment, the one or more energy emitters 461 include at least one two-photon excitation component. In an embodiment, the one or more energy emitters 461 include at least one of an exciplex laser, a diode-pumped solid state laser, or a semiconductor laser.

Further non-limiting examples of energy emitters 461 include radiation emitters, ion emitters, photon emitters, electron emitters, gamma emitters, or the like. In an embodiment, the one or more energy emitters 461 include one or more incandescent emitters, transducers, heat sources, or continuous wave bulbs. In an embodiment, the one or more energy emitters 461 include one or more laser, light-emitting diodes, laser diodes, fiber lasers, lasers, or ultra-fast lasers, quantum dots, organic light-emitting diodes, microcavity light-emitting diodes, or polymer light-emitting diodes. Further non-limiting examples of energy emitters 461 include electromagnetic energy emitters. In an embodiment, the implantable device 102 includes one or more electromagnetic energy emitters. In an embodiment, the one or more electromagnetic energy emitters provide a voltage across a portion of CSF received within one or more fluid-flow passageways 108. In an embodiment, the one or more electromagnetic energy emitters include one or more electrodes. In an embodiment, the one or more electromagnetic energy emitters include one or more light-emitting diodes. In an embodiment, the one or more electromagnetic energy emitters include at least one electron emitting material.

FIG. 4C show an implantable wireless biotelemetry device 470, in which one or more methodologies or technologies can be implemented such as, for example, reporting a neuropsychiatric disorder status information. In an embodiment, the implantable wireless biotelemetry device 470 includes a sensor component 204 configured to detect at least one biomarker profile of CSF received within one or more fluid-flow passageways 108 of the implantable wireless biotelemetry device 470.

In an embodiment, the implantable wireless biotelemetry device 470 includes one or more computer-readable memory media 215 including executable instructions stored thereon that, when executed on a computer, instruct a computing device 208 to execute one or more protocols. For example, in an embodiment, the implantable wireless biotelemetry device 470 includes one or more computer-readable memory media 215 including executable instructions stored thereon that, when executed on a computer, instruct a computing device 208 to retrieve from storage one or more parameters associated with reference CSF biomarker spectral information associated with at least one neuropsychiatric disorder, and to perform a comparison of a detected at least one biomarker profile to the retrieved one or more parameters. In an embodiment, the reference CSF biomarker spectral information associated with the at least one neuropsychiatric disorder includes CSF psychiatric disorder biomarker spectral information. In an embodiment, the reference CSF biomarker spectral information associated with the at least one neuropsychiatric disorder includes CSF bipolar disorder biomarker spectral information. In an embodiment, the reference CSF biomarker spectral information associated with the at least one neuropsychiatric disorder includes CSF mood disorder biomarker spectral information. In an embodiment, the reference CSF biomarker spectral information associated with the at least one neuropsychiatric disorder includes CSF chronic dementia disease biomarker spectral information.

In an embodiment, the one or more computer-readable memory media 215 further include executable instructions stored thereon that, when executed on a computer, instruct a computing device 208 to generate neuropsychiatric disorder status information in response to the comparison. In an embodiment, the one or more computer-readable memory media 215 further include executable instructions stored thereon that, when executed on a computer, instruct a computing device 208 to generate a schizophrenia spectrum diagnosis in response to the comparison. In an embodiment, the one or more computer-readable memory media 215 further include executable instructions stored thereon that, when executed on a computer, instruct a computing device 208 to cause the storing of neurobiological change information in response to the comparison. In an embodiment, the one or more computer-readable memory media 215 further include executable instructions stored thereon that, when executed on a computer, instruct a computing device 208 to generate an affective disorder score in response to the comparison.

In an embodiment, the implantable wireless biotelemetry device 470 includes a transceiver 207 operable to concurrently or sequentially transmit or receive information in response to the comparison of a detected at least one biomarker profile to the retrieved one or more parameters. In an embodiment, the transceiver 207 reports status information at a plurality of time intervals in response to the comparison. In an embodiment, wherein the transceiver 207 reports status information at a plurality of time intervals and to enter a receive mode for a period after transmitting the report information. In an embodiment, the transceiver 207 requests CSF biomarker spectral information in response to the comparison.

In an embodiment, the implantable wireless biotelemetry device 470 is configure to monitor CSF neurological disorder biomarkers or CSF psychiatric disorder biomarkers and to send, receive, or store information associated with the monitoring of the CSF neurological disorder biomarkers or CSF psychiatric disorder biomarkers. In an embodiment, the implantable wireless biotelemetry device 470 includes a biomarker telematic information generation circuit 482 and a telematic biomarker reporter circuit 484. In an embodiment, the biomarker telematic information generation circuit 482 generates biomarker telematic information associated with at least one in vivo detected CSF neurological disorder biomarker or CSF psychiatric disorder biomarker. In an embodiment, the telematic biomarker reporter circuit 484 transmits at least one of neurological disorder biomarker information or CSF psychiatric disorder biomarker information.

In an embodiment, the implantable wireless biotelemetry device 470 is configured to generate time-series information associated with CSF applied to an array. For example, in an embodiment, time-series information of CSF assays is generated by contacting a protein-analytic micro-array of the implantable wireless biotelemetry device 470 to CSF. In an embodiment, the implantable wireless biotelemetry device 470 includes an array that is read-out telemetrically (e.g., upon interrogation) to diagnose the progress-in-time of a progressive condition based on detected cerebrospinal fluid compositional information.

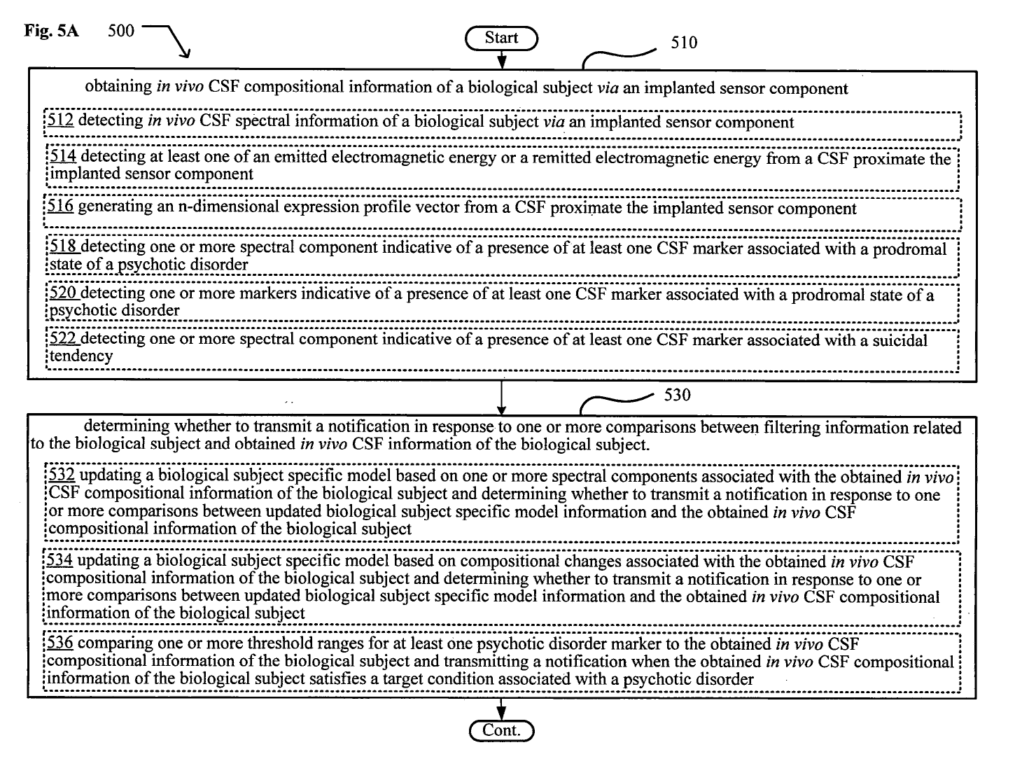

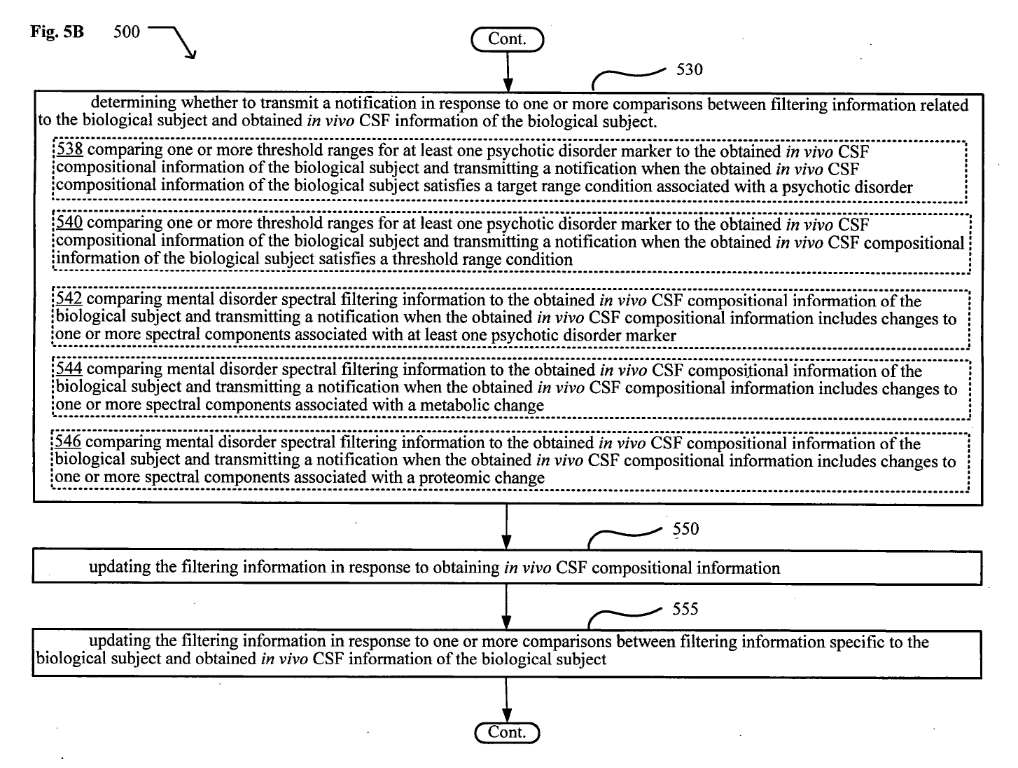

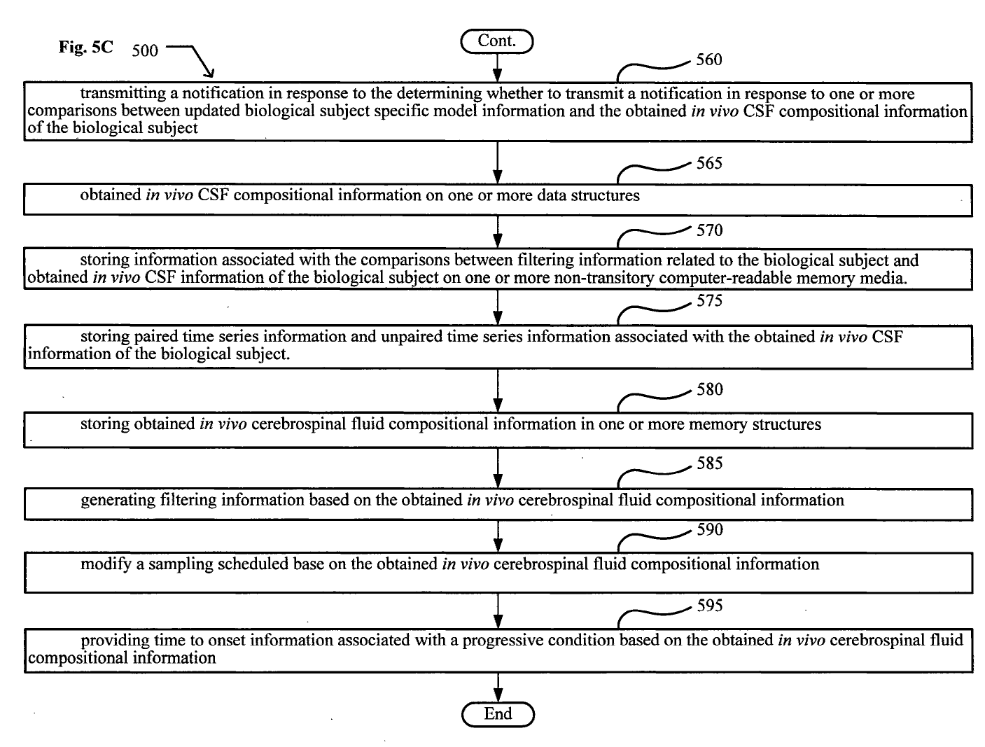

FIGS. 5A, 5B, and 5C show an example of a real-time monitoring method 500. At 510, the method 500 includes obtaining in vivo CSF compositional information of a biological subject via an implanted sensor component 204. At 512, obtaining the in vivo CSF compositional information of the biological subject via the implanted sensor component 204 includes detecting in vivo CSF spectral information of a biological subject via the implanted sensor component 204. At 514, obtaining the in vivo CSF compositional information of the biological subject via the implanted sensor component 204 includes detecting at least one of an emitted electromagnetic energy or a remitted electromagnetic energy from CSF proximate the implanted sensor component 204. At 516, obtaining the in vivo CSF compositional information of the biological subject via the implanted sensor component 204 includes generating an n-dimensional expression profile vector from CSF proximate the implanted sensor component 204. At 518, obtaining the in vivo CSF compositional information of the biological subject via the implanted sensor component 204 includes detecting one or more spectral components indicative of a presence of at least one CSF marker associated with a prodromal state of a psychotic disorder. At 520, obtaining the in vivo CSF compositional information of the biological subject via the implanted sensor component 204 includes detecting one or more markers indicative of a presence of at least one CSF marker associated with a prodromal state of a psychotic disorder.

At 522, obtaining the in vivo CSF compositional information of the biological subject via the implanted sensor component 204 includes detecting one or more spectral components indicative of a presence of at least one CSF marker associated with a suicidal tendency. In an embodiment, obtaining the in vivo CSF compositional information of the biological subject via the implanted sensor component 204 includes detecting one or more spectral components indicative of a presence of at least one cerebrospinal fluid marker associated with a state of a psychotic disorder. In an embodiment, obtaining the in vivo CSF compositional information of the biological subject via the implanted sensor component 204 includes detecting one or more markers indicative of a presence of at least one cerebrospinal fluid marker associated with a state of a psychotic disorder. In an embodiment, obtaining the in vivo CSF compositional information of the biological subject via the implanted sensor component 204 includes detecting one or more cerebrospinal fluid markers associated with a suicidal tendency. In an embodiment, obtaining the in vivo CSF compositional information of the biological subject via the implanted sensor component 204 includes detecting one or more binding events indicative of a presence of at least one cerebrospinal fluid marker associated with a state of a psychotic disorder.

At 530, the method 500 includes determining whether to transmit a notification in response to one or more comparisons between filtering information 234 related to the biological subject and obtained in vivo CSF information of the biological subject. At 532, determining whether to transmit the notification in response to the one or more comparisons includes updating a biological subject specific model based on one or more spectral components associated with the obtained in vivo CSF compositional information of the biological subject and determining whether to transmit a notification in response to one or more comparisons between updated biological subject specific model information and the obtained in vivo CSF compositional information of the biological subject. At 534, determining whether to transmit the notification in response to the one or more comparisons includes updating a biological subject specific model based on compositional changes associated with the obtained in vivo CSF compositional information of the biological subject and determining whether to transmit a notification in response to one or more comparisons between updated biological subject specific model information and the obtained in vivo CSF compositional information of the biological subject.

At 536, determining whether to transmit the notification in response to the one or more comparisons includes comparing one or more threshold ranges for at least one psychotic disorder marker to the obtained in vivo CSF compositional information of the biological subject and transmitting a notification when the obtained in vivo CSF compositional information of the biological subject satisfies a target condition associated with a psychotic disorder. In an embodiment, determining whether to transmit the notification in response to the one or more comparisons includes comparing one or more threshold ranges for at least one psychotic disorder marker to the obtained in vivo CSF compositional information of the biological subject and transmitting a notification when the obtained in vivo CSF compositional information of the biological subject meets or exceeds a target range. At 538, determining whether to transmit the notification in response to the one or more comparisons includes comparing one or more threshold ranges for at least one psychotic disorder marker to the obtained in vivo CSF compositional information of the biological subject and transmitting a notification when the obtained in vivo CSF compositional information of the biological subject satisfies a target range condition associated with a psychotic disorder.

At 540, determining whether to transmit the notification in response to the one or more comparisons includes comparing one or more threshold ranges for at least one psychotic disorder marker to the obtained in vivo CSF compositional information of the biological subject and transmitting a notification when the obtained in vivo CSF compositional information of the biological subject satisfies a threshold range condition. At 542, determining whether to transmit the notification in response to the one or more comparisons includes comparing mental disorder spectral filtering information to the obtained in vivo CSF compositional information of the biological subject and transmitting a notification when the obtained in vivo CSF compositional information includes changes to one or more spectral components associated with at least one psychotic disorder marker. At 544, determining whether to transmit the notification in response to the one or more comparisons includes comparing mental disorder spectral filtering information to the obtained in vivo CSF compositional information of the biological subject and transmitting a notification when the obtained in vivo CSF compositional information includes changes to one or more spectral components associated with a metabolic change.

At 546, determining whether to transmit the notification in response to the one or more comparisons includes comparing mental disorder spectral filtering information to the obtained in vivo CSF compositional information of the biological subject and transmitting a notification when the obtained in vivo CSF compositional information includes changes to one or more spectral components associated with a proteomic change. At 550, the method 500 includes updating the filtering information in response to obtaining in vivo CSF compositional information. At 555, the method 500 includes updating the filtering information in response to one or more comparisons between filtering information specific to the biological subject and obtained in vivo CSF information of the biological subject. At 560, the method 500 includes transmitting a notification in response to the determining whether to transmit a notification in response to one or more comparisons between updated biological subject specific model information and the obtained in vivo CSF compositional information of the biological subject. At 565, the method 500 includes storing obtained in vivo CSF compositional information on one or more data structures 230. At 570, the method 500 includes storing information associated with the comparisons between filtering information related to the biological subject and obtained in vivo CSF information of the biological subject on one or more non-transitory computer-readable memory media 215. At 575, the method 500 includes storing paired time series information and unpaired time series information associated with the obtained in vivo CSF information of the biological subject.

At 580, the method 500 includes storing obtained in vivo cerebrospinal fluid compositional information in one or more memory structures. At 585, the method 500 includes generating filtering information based on the obtained in vivo cerebrospinal fluid compositional information. At 590, the method 500 includes modify a sampling scheduled base on the obtained in vivo cerebrospinal fluid compositional information. At 595, the method 500 includes providing time to onset information associated with a progressive condition based on the obtained in vivo cerebrospinal fluid compositional information.

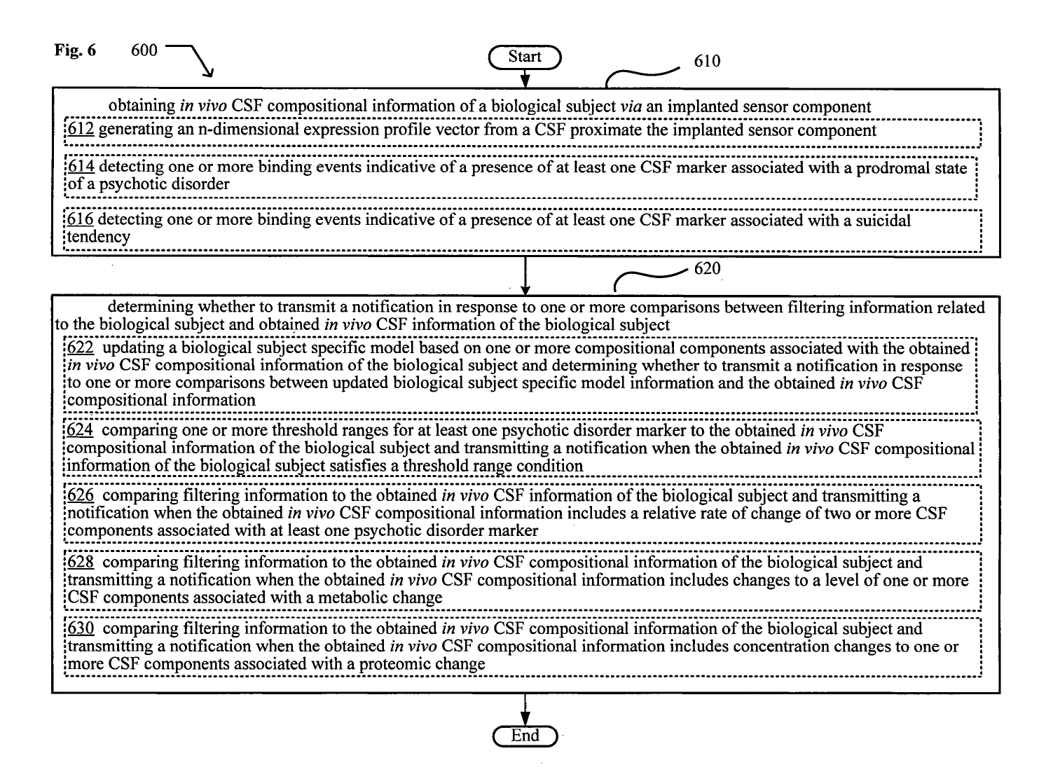

FIG. 6 shows an example of a real-time monitoring method 600. At 610, the method 600 includes obtaining in vivo CSF compositional information of a biological subject via an implanted sensor component 204. At 612, obtaining the in vivo CSF compositional information of the biological subject via the implanted sensor component 204 includes generating an n-dimensional expression profile vector from CSF proximate the implanted sensor component 204. At 614, obtaining the in vivo CSF compositional information of the biological subject via the implanted sensor component 204 includes detecting one or more binding events indicative of a presence of at least one CSF marker associated with a prodromal state of a psychotic disorder. At 616, obtaining the in vivo CSF compositional information of the biological subject via the implanted sensor component 204 includes detecting one or more binding events indicative of a presence of at least one CSF marker associated with a suicidal tendency.

At 620, the method 600 includes determining whether to transmit a notification in response to one or more comparisons between filtering information related to the biological subject and obtained in vivo CSF information of the biological subject. At 622, determining whether to transmit the notification in response to the one or more comparisons includes updating a biological subject specific model based on one or more compositional components associated with the obtained in vivo CSF compositional information of the biological subject and determining whether to transmit a notification in response to one or more comparisons between updated biological subject specific model information and the obtained in vivo CSF compositional information. At 624, determining whether to transmit the notification in response to the one or more comparisons includes comparing one or more threshold ranges for at least one psychotic disorder marker to the obtained in vivo CSF compositional information of the biological subject and transmitting a notification when the obtained in vivo CSF compositional information of the biological subject satisfies a threshold range condition. At 626, determining whether to transmit the notification in response to the one or more comparisons includes comparing filtering information to the obtained in vivo CSF information of the biological subject and transmitting a notification when the obtained in vivo CSF compositional information includes a relative rate of change of two or more CSF components associated with at least one psychotic disorder marker.

At 628, determining whether to transmit the notification in response to the one or more comparisons includes comparing filtering information to the obtained in vivo CSF compositional information of the biological subject and transmitting a notification when the obtained in vivo CSF compositional information includes changes to a level of one or more CSF components associated with a metabolic change. At 630, determining whether to transmit the notification in response to the one or more comparisons includes comparing filtering information to the obtained in vivo CSF compositional information of the biological subject and transmitting a notification when the obtained in vivo CSF compositional information includes concentration changes to one or more CSF components associated with a proteomic change.

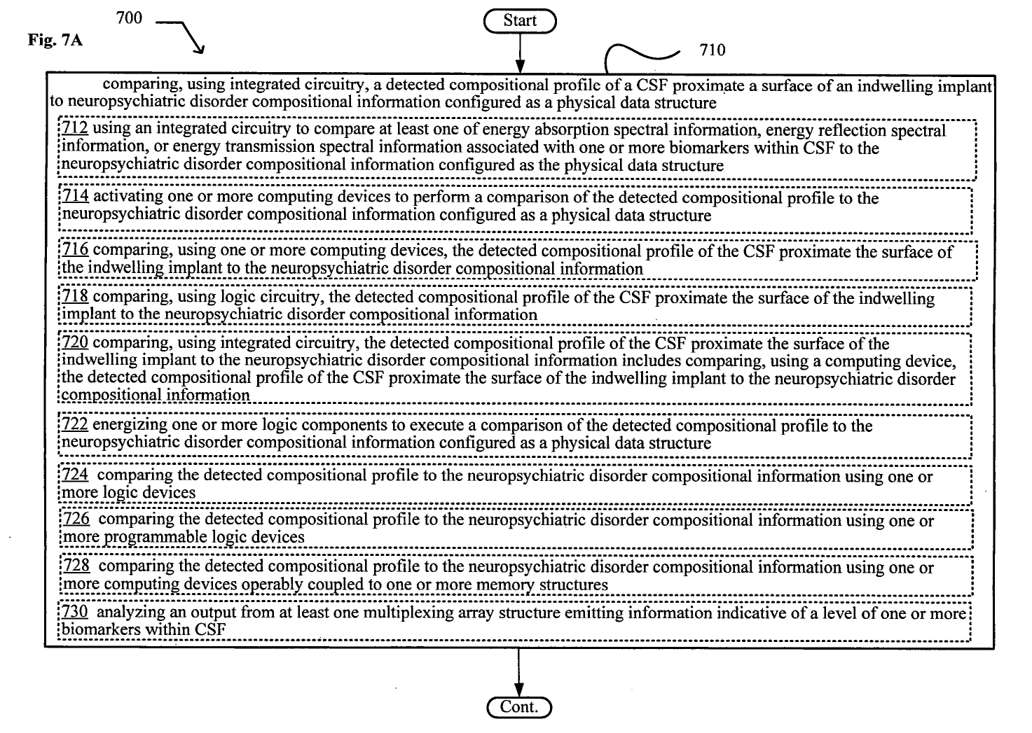

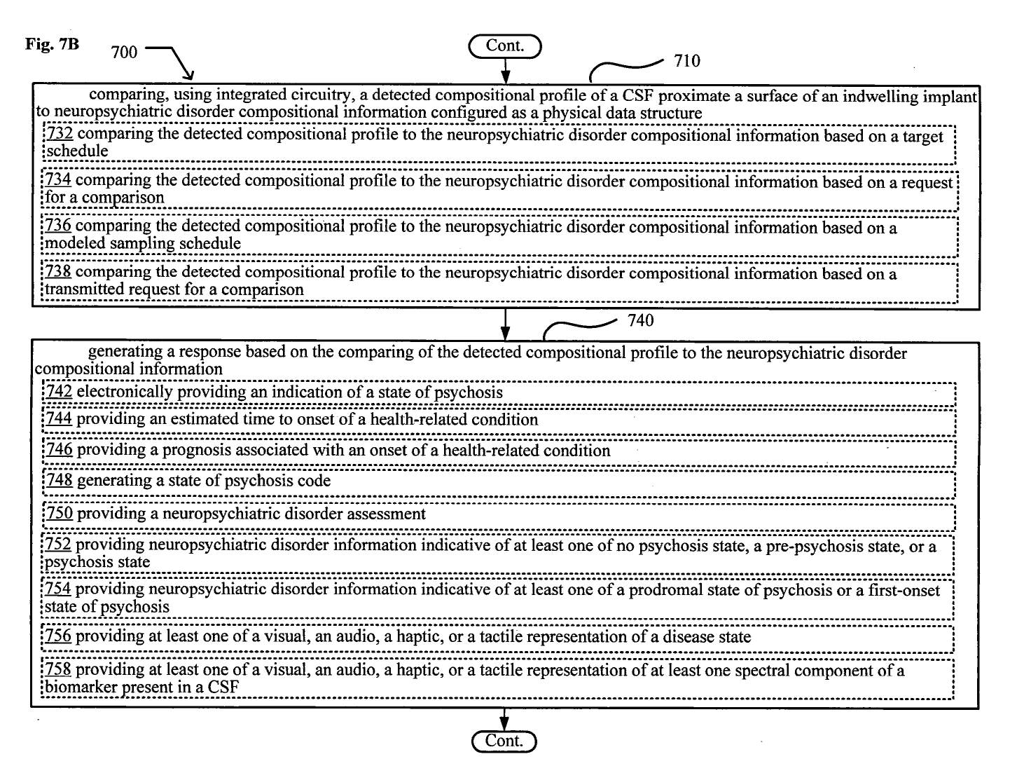

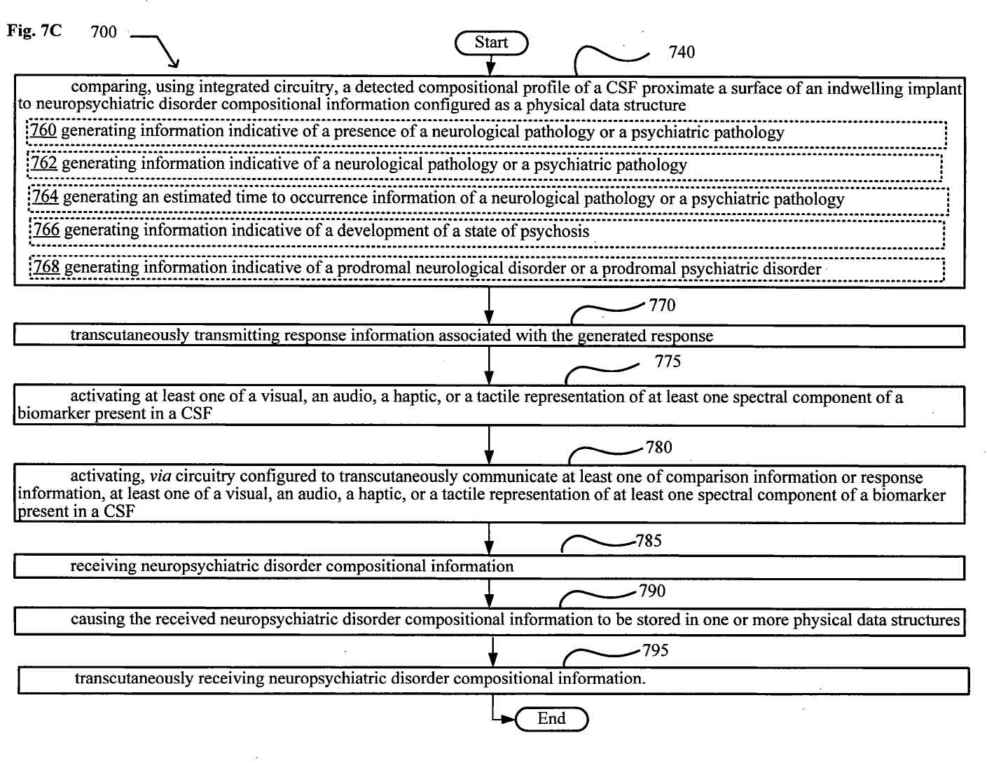

FIG. 7 shows an in vivo method 700 for real-time monitoring of one or more biomarkers within CSF. At 710, the method 700 includes comparing, using integrated circuitry, a detected compositional profile of CSF proximate a surface of an indwelling implant to neuropsychiatric disorder compositional information configured as a physical data structure 230. At 712, comparing, using integrated circuitry, the detected compositional profile of the CSF proximate the surface of the indwelling implant to the neuropsychiatric disorder compositional information includes using an integrated circuitry to compare at least one of energy absorption spectral information, energy reflection spectral information, or energy transmission spectral information associated with one or more biomarkers within CSF to the neuropsychiatric disorder compositional information configured as the physical data structure 230. At 714, comparing, using integrated circuitry, the detected compositional profile of the CSF proximate the surface of the indwelling implant to the neuropsychiatric disorder compositional information includes activating one or more computing devices 208 to perform a comparison of the detected compositional profile to the neuropsychiatric disorder compositional information configured as a physical data structure 230. At 716, comparing, using integrated circuitry, the detected compositional profile of the CSF proximate the surface of the indwelling implant to the neuropsychiatric disorder compositional information includes comparing, using one or more computing devices 208, the detected compositional profile of the CSF proximate the surface of the indwelling implant to the neuropsychiatric disorder compositional information.

At 718, comparing, using integrated circuitry, the detected compositional profile of the CSF proximate the surface of the indwelling implant to the neuropsychiatric disorder compositional information includes comparing, using logic circuitry, the detected compositional profile of the CSF proximate the surface of the indwelling implant to the neuropsychiatric disorder compositional information. At 720, comparing, using integrated circuitry, the detected compositional profile of the CSF proximate the surface of the indwelling implant to the neuropsychiatric disorder compositional information includes comparing, using a computing device, the detected compositional profile of the CSF proximate the surface of the indwelling implant to the neuropsychiatric disorder compositional information. At 722, comparing, using integrated circuitry, the detected compositional profile of the CSF proximate the surface of the indwelling implant to the neuropsychiatric disorder compositional information includes energizing one or more logic components to execute a comparison of the detected compositional profile to the neuropsychiatric disorder compositional information configured as a physical data structure 230.

At 724, comparing, using integrated circuitry, the detected compositional profile of the CSF proximate the surface of the indwelling implant to the neuropsychiatric disorder compositional information includes comparing the detected compositional profile to the neuropsychiatric disorder compositional information using one or more logic devices. At 726, comparing, using integrated circuitry, the detected compositional profile of the CSF proximate the surface of the indwelling implant to the neuropsychiatric disorder compositional information includes comparing the detected compositional profile to the neuropsychiatric disorder compositional information using one or more programmable logic devices. At 728, comparing, using integrated circuitry, the detected compositional profile of the CSF proximate the surface of the indwelling implant to the neuropsychiatric disorder compositional information includes comparing the detected compositional profile to the neuropsychiatric disorder compositional information using one or more computing devices operably coupled to one or more memory structures.

At 730, comparing, using integrated circuitry, the detected compositional profile of the CSF proximate the surface of the indwelling implant to the neuropsychiatric disorder compositional information includes analyzing an output from at least one multiplexing array structure emitting information indicative of a level of one or more biomarkers within CSF. At 732, comparing, using integrated circuitry, the detected compositional profile of the CSF proximate the surface of the indwelling implant to the neuropsychiatric disorder compositional information includes comparing the detected compositional profile to the neuropsychiatric disorder compositional information based on a target schedule. At 734, comparing, using integrated circuitry, the detected compositional profile of the CSF proximate the surface of the indwelling implant to the neuropsychiatric disorder compositional information includes comparing the detected compositional profile to the neuropsychiatric disorder compositional information based on a request for a comparison.

At 736, comparing, using integrated circuitry, the detected compositional profile of the CSF proximate the surface of the indwelling implant to the neuropsychiatric disorder compositional information includes comparing the detected compositional profile to the neuropsychiatric disorder compositional information based on a modeled sampling schedule. At 738, comparing, using integrated circuitry, the detected compositional profile of the CSF proximate the surface of the indwelling implant to the neuropsychiatric disorder compositional information includes comparing the detected compositional profile to the neuropsychiatric disorder compositional information based on a transmitted request for a comparison. In an embodiment, comparing, using integrated circuitry, the detected compositional profile includes comparing detected optical energy absorption profile of the CSF proximate the surface of the indwelling implant to neuropsychiatric disorder spectral information. At 740, the method 700 includes generating a response based on the comparing of the detected compositional profile to the neuropsychiatric disorder compositional information. At 742, generating the response includes electronically providing an indication of a state of psychosis. At 744, generating the response includes providing an estimated time to onset of a health-related condition. At 746, generating the response includes providing a prognosis associated with an onset of a health-related condition. At 748, generating the response includes generating a state of psychosis code.

At 750, generating the response includes providing a neuropsychiatric disorder assessment. At 752, generating the response includes electronically providing neuropsychiatric disorder information indicative of at least one of no psychosis state, a pre-psychosis state, or a psychosis state. At 754, generating the response includes providing neuropsychiatric disorder information indicative of at least one of a prodromal state of psychosis or a first-onset state of psychosis. At 756, generating the response includes providing at least one of a visual, an audio, a haptic, or a tactile representation of a disease state.

At 758, generating the response includes providing at least one of a visual, an audio, a haptic, or a tactile representation of at least one spectral component of a biomarker present in CSF. At 760, generating the response includes generating information indicative of a presence of a neurological pathology or a psychiatric pathology. At 762, generating the response includes generating information indicative of a neurological pathology or a psychiatric pathology. At 764, generating the response includes generating an estimated time to occurrence information of a neurological pathology or a psychiatric pathology. At 766, generating the response includes generating information indicative of a development of a state of psychosis. At 768, generating the response includes generating information indicative of a prodromal neurological disorder or a prodromal psychiatric disorder.

At 770, the method 700 includes transcutaneously transmitting response information associated with the generated response. At 775, the method 700 includes activating at least one of a visual, an audio, a haptic, or a tactile representation of at least one spectral component of a biomarker present in CSF. At 780, the method 700 includes activating, via circuitry configured to transcutaneously communicate at least one of comparison information or response information, at least one of a visual, an audio, a haptic, or a tactile representation of at least one spectral component of a biomarker present in CSF. At 785, the method 700 includes receiving neuropsychiatric disorder compositional information. At 790, the method 700 includes causing the received neuropsychiatric disorder compositional information to be stored in one or more physical data structures 230. At 795, the method 700 includes transcutaneously receiving neuropsychiatric disorder compositional information.

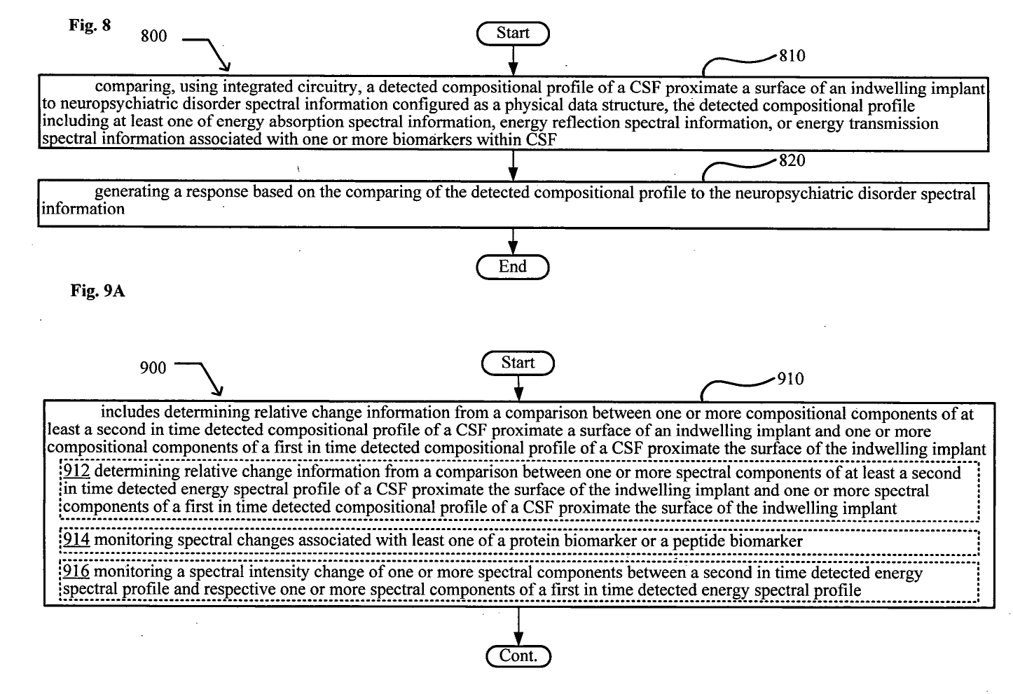

FIG. 8 shows an in vivo real-time monitoring method 800. At 810, the method 800 includes comparing, using integrated circuitry, a detected compositional profile of CSF proximate a surface of an indwelling implant to neuropsychiatric disorder spectral information configured as a physical data structure 230, the detected compositional profile including at least one of energy absorption spectral information, energy reflection spectral information, or energy transmission spectral information associated with one or more biomarkers within CSF. At 820, the method 800 includes generating a response based on the comparing of the detected compositional profile to the neuropsychiatric disorder spectral information.

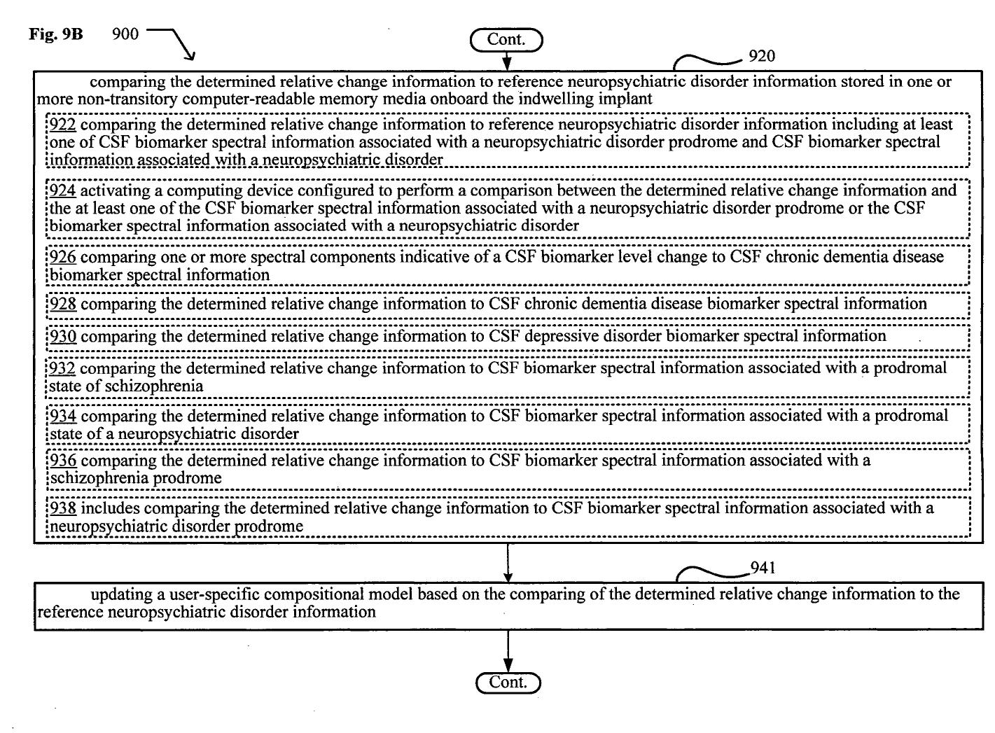

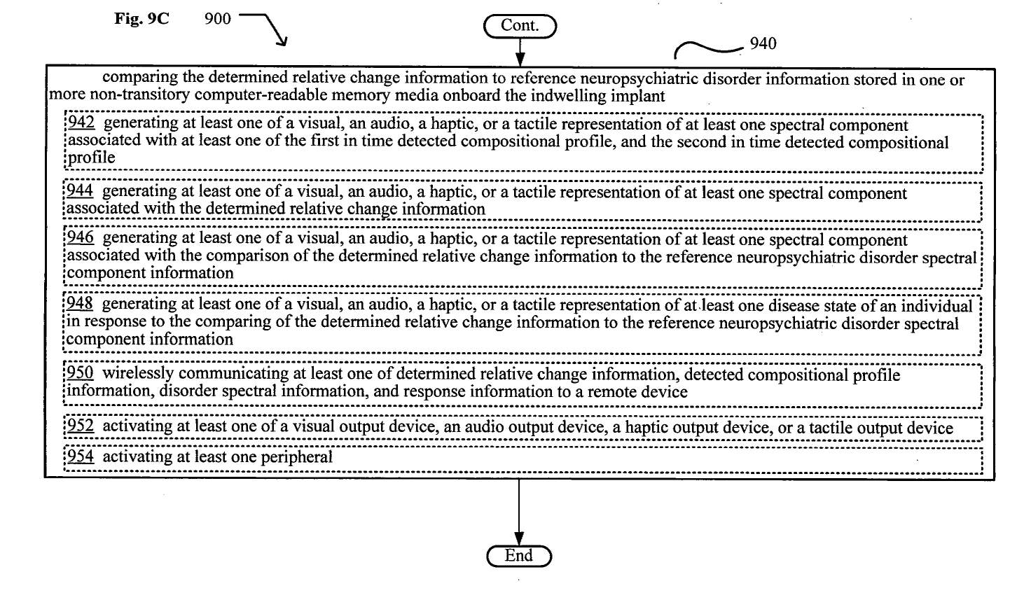

FIGS. 9A, 9B, and 9C show an in vivo real-time monitoring method 900. At 910, the method 900 includes determining relative change information from a comparison between one or more compositional components of at least a second in time detected compositional profile of CSF proximate a surface of an indwelling implant and one or more compositional components of a first in time detected compositional profile of CSF proximate the surface of the indwelling implant. At 912, determining the relative change information includes determining relative change information from a comparison between one or more spectral components of at least a second in time detected energy spectral profile of CSF proximate the surface of the indwelling implant and one or more spectral components of a first in time detected compositional profile of CSF proximate the surface of the indwelling implant. At 914, determining relative change information includes monitoring spectral changes associated with least one of a protein biomarker or a peptide biomarker. At 916, determining relative change information includes monitoring a spectral intensity change of one or more spectral components between a second in time detected energy spectral profile and respective one or more spectral components of a first in time detected energy spectral profile.

At 920, the method 900 includes comparing the determined relative change information to reference neuropsychiatric disorder information stored in one or more non-transitory computer-readable memory media 215 onboard the indwelling implant. At 922, comparing the determined relative change information to the reference neuropsychiatric disorder information includes comparing the determined relative change information to reference neuropsychiatric disorder information including at least one of CSF biomarker spectral information associated with a neuropsychiatric disorder prodrome and CSF biomarker spectral information associated with a neuropsychiatric disorder. In an embodiment, comparing the determined relative change information to the reference neuropsychiatric disorder information includes comparing the determined relative change information to user-specific reference neuropsychiatric disorder information.

At 924, comparing the determined relative change information to the reference neuropsychiatric disorder information includes activating a computing device configured to perform a comparison between the determined relative change information and the at least one of the CSF biomarker spectral information associated with a neuropsychiatric disorder prodrome or the CSF biomarker spectral information associated with a neuropsychiatric disorder.

At 926, comparing the determined relative change information to the reference neuropsychiatric disorder information includes comparing one or more spectral components indicative of a CSF biomarker level change to CSF chronic dementia disease biomarker spectral information. At 928, comparing the determined relative change information to the reference neuropsychiatric disorder information includes comparing the determined relative change information to CSF chronic dementia disease biomarker spectral information. At 930, comparing the determined relative change information to the reference neuropsychiatric disorder information includes comparing the determined relative change information to CSF depressive disorder biomarker spectral information. At 932, comparing the determined relative change information to the reference neuropsychiatric disorder information includes comparing the determined relative change information to CSF biomarker spectral information associated with a prodromal state of schizophrenia.

At 934, comparing the determined relative change information to the reference neuropsychiatric disorder information includes comparing the determined relative change information to CSF biomarker spectral information associated with a prodromal state of a neuropsychiatric disorder. At 936, comparing the determined relative change information to the reference neuropsychiatric disorder information includes comparing the determined relative change information to CSF biomarker spectral information associated with a schizophrenia prodrome. At 938, comparing the determined relative change information to the reference neuropsychiatric disorder information includes comparing the determined relative change information to CSF biomarker spectral information associated with a neuropsychiatric disorder prodrome.

At 941, the method 900 includes updating a user-specific compositional model based on the comparing of the determined relative change information to the reference neuropsychiatric disorder information. At 940, the method 900 includes generating a response based on the comparing of the determined relative change information to the reference neuropsychiatric disorder compositional information. At 942, generating the response includes generating at least one of a visual, an audio, a haptic, or a tactile representation of at least one spectral component associated with at least one of the first in time detected compositional profile, and the second in time detected compositional profile. At 944, generating the response includes generating at least one of a visual, an audio, a haptic, or a tactile representation of at least one spectral component associated with the determined relative change information. At 946, generating the response includes generating at least one of a visual, an audio, a haptic, or a tactile representation of at least one spectral component associated with the comparison of the determined relative change information to the reference neuropsychiatric disorder spectral component information. At 948, generating the response includes generating at least one of a visual, an audio, a haptic, or a tactile representation of at least one disease state of an individual in response to the comparing of the determined relative change information to the reference neuropsychiatric disorder spectral component information. At 950, generating the response includes wirelessly communicating at least one of determined relative change information, detected compositional profile information, disorder spectral information, and response information to a remote device. At 952, generating the response includes activating at least one of a visual output device, an audio output device, a haptic output device, or a tactile output device. At 954, generating the response includes activating at least one peripheral.

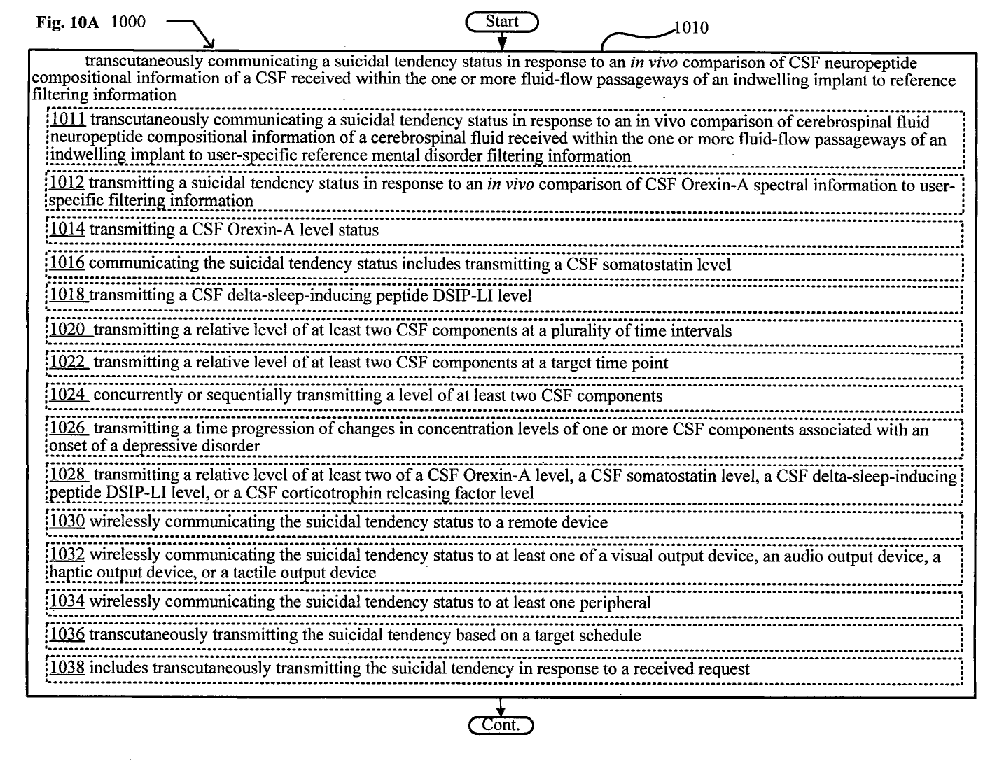

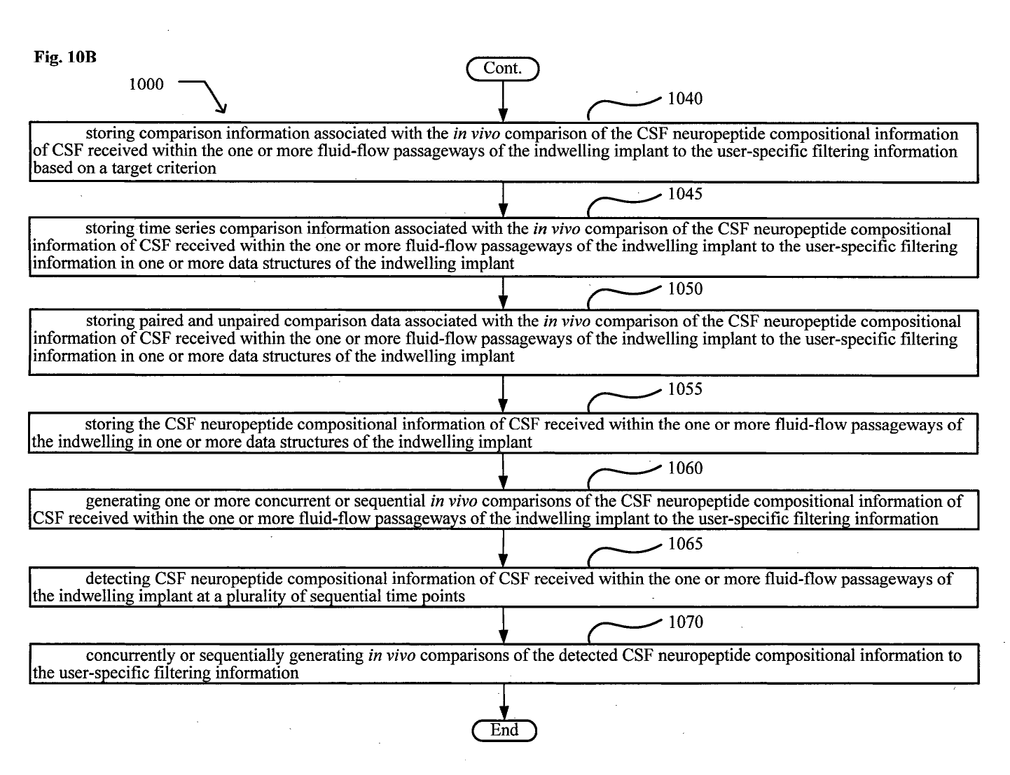

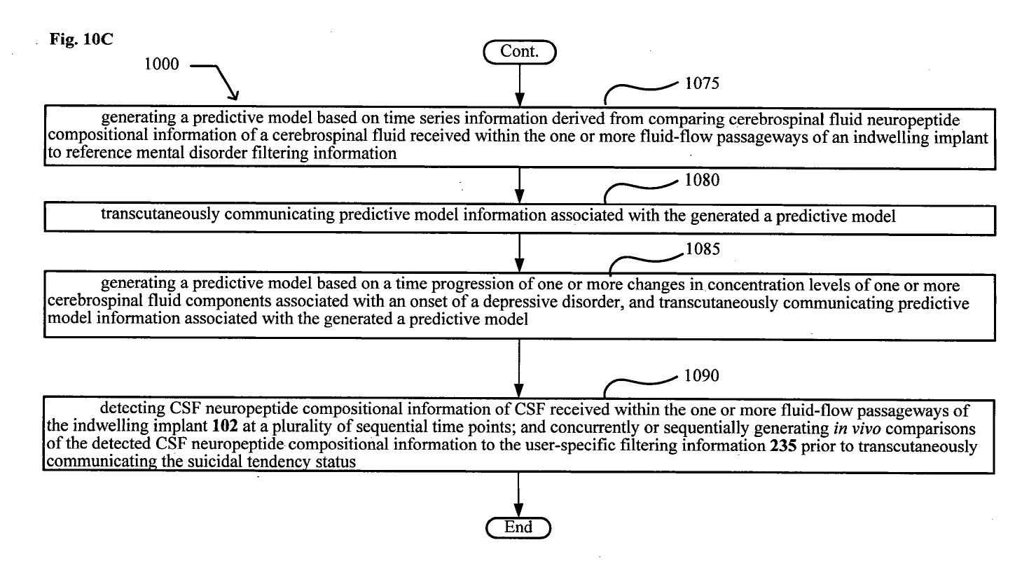

FIGS. 10A, 10B, and 10C show a method 1000 for predicting an onset of a depressive disorder. At 1010, the method 1000 includes transcutaneously communicating a suicidal tendency status in response to an in vivo comparison of CSF neuropeptide compositional information of CSF received within the one or more fluid-flow passageways of an indwelling implant 102 to reference filtering information. At 1011, transcutaneously communicating the suicidal tendency status includes transcutaneously communicating a suicidal tendency status in response to an in vivo comparison of cerebrospinal fluid neuropeptide compositional information of a cerebrospinal fluid received within the one or more fluid-flow passageways of an indwelling implant to user-specific reference mental disorder filtering information. At 1012, transcutaneously communicating the suicidal tendency status includes transmitting a suicidal tendency status in response to an in vivo comparison of CSF Orexin-A spectral information to user-specific filtering information 235.

At 1014, transcutaneously communicating the suicidal tendency status includes transmitting a CSF Orexin-A level status. At 1016, transcutaneously communicating the suicidal tendency status includes transmitting a CSF somatostatin level. At 1018, transcutaneously communicating the suicidal tendency status includes transmitting a CSF delta-sleep-inducing peptide DSIP-LI level. At 1020, transcutaneously communicating the suicidal tendency status includes transmitting a relative level of at least two CSF components at a plurality of time intervals. At 1022, transcutaneously communicating the suicidal tendency status includes transmitting a relative level of at least two CSF components at a target time point. At 1024, transcutaneously communicating the suicidal tendency status includes concurrently or sequentially transmitting a level of at least two CSF components. At 1026, transcutaneously communicating the suicidal tendency status includes transmitting a time progression of changes in concentration levels of one or more CSF components associated with an onset of a depressive disorder. In an embodiment, transcutaneously communicating the suicidal tendency status includes transmitting predictive model information generated based on a time progression of detected changes in concentration levels of one or more cerebrospinal fluid components associated with an onset of a depressive disorder.

At 1028, transcutaneously communicating the suicidal tendency status includes transmitting a relative level of at least two of a CSF Orexin-A level, a CSF somatostatin level, a CSF delta-sleep-inducing peptide DSIP-LI level, or a CSF corticotrophin releasing factor level. At 1030, transcutaneously communicating the suicidal tendency status includes wirelessly communicating the suicidal tendency status to a remote device. At 1032, transcutaneously communicating the suicidal tendency status includes wirelessly communicating the suicidal tendency status to at least one of a visual output device, an audio output device, a haptic output device, or a tactile output device. At 1034, transcutaneously communicating the suicidal tendency status includes wirelessly communicating the suicidal tendency status to at least one peripheral. At 1036, transcutaneously communicating the suicidal tendency status includes transcutaneously transmitting the suicidal tendency based on a target schedule. At 1038, transcutaneously communicating the suicidal tendency status includes transcutaneously transmitting the suicidal tendency in response to a received request.

At 1040, the method 1000 includes storing comparison information associated with the in vivo comparison of the CSF neuropeptide compositional information of CSF received within the one or more fluid-flow passageways of the indwelling implant 102 to the user-specific filtering information 235 based on a target criterion. At 1045, the method 1000 includes storing time series comparison information associated with the in vivo comparison of the CSF neuropeptide compositional information of CSF received within the one or more fluid-flow passageways of the indwelling implant 102 to the user-specific filtering information 235 in one or more data structures 230 of the indwelling implant 102. At 1050, the method 1000 includes storing paired and unpaired comparison data associated with the in vivo comparison of the CSF neuropeptide compositional information of CSF received within the one or more fluid-flow passageways of the indwelling implant 102 to the user-specific filtering information 235 in one or more data structures 230 of the indwelling implant 102.

At 1055, the method 1000 includes storing the CSF neuropeptide compositional information of CSF received within the one or more fluid-flow passageways of the indwelling in one or more data structures 230 of the indwelling implant 102. At 1060, the method 1000 includes generating one or more concurrent or sequential in vivo comparisons of the CSF neuropeptide compositional information of CSF received within the one or more fluid-flow passageways of the indwelling implant 102 to the user-specific filtering information 235. At 1065, the method 1000 includes detecting CSF neuropeptide compositional information of CSF received within the one or more fluid-flow passageways of the indwelling implant 102 at a plurality of sequential time points. At 1070, the method 1000 includes concurrently or sequentially generating in vivo comparisons of the detected CSF neuropeptide compositional information to the user-specific filtering information 235.

At 1075, the method 1000 includes generating a predictive model based on time series information derived from comparing cerebrospinal fluid neuropeptide compositional information of a cerebrospinal fluid received within the one or more fluid-flow passageways of an indwelling implant to reference mental disorder filtering information. At 1080, the method 1000 includes transcutaneously communicating predictive model information associated with the generated a predictive model. At 1085, the method 1000 includes generating a predictive model based on a time progression of one or more changes in concentration levels of one or more cerebrospinal fluid components associated with an onset of a depressive disorder, and transcutaneously communicating predictive model information associated with the generated a predictive model. At 1090, the method 1000 includes detecting CSF neuropeptide compositional information of CSF received within the one or more fluid-flow passageways of the indwelling implant 102 at a plurality of sequential time points; and concurrently or sequentially generating in vivo comparisons of the detected CSF neuropeptide compositional information to the user-specific filtering information 235 prior to transcutaneously communicating the suicidal tendency status.

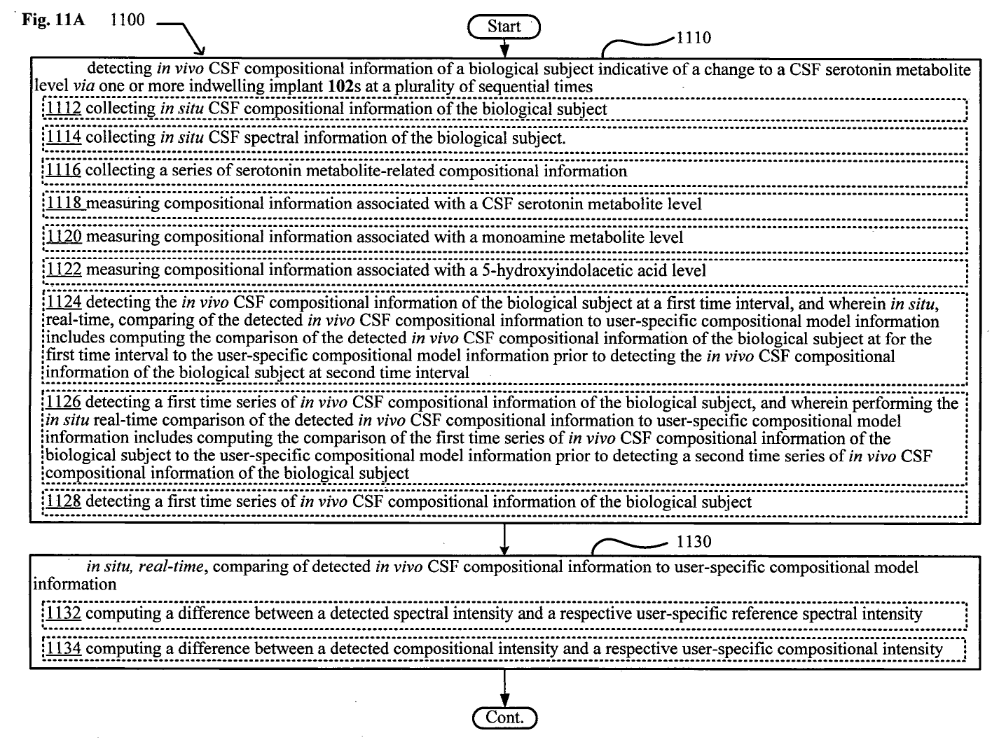

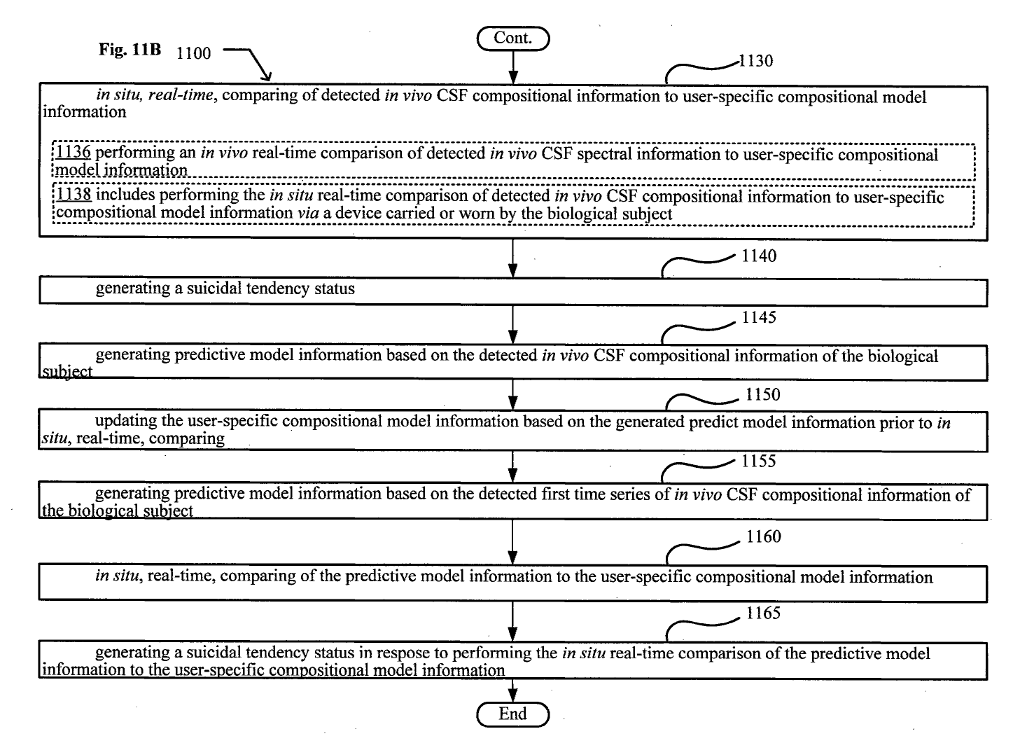

FIGS. 11A and 11B show a method 1100 for monitoring CSF biomarkers indicative of suicidal tendencies. At 1110, the method 1100 includes detecting in vivo CSF compositional information of a biological subject indicative of a change to a CSF serotonin metabolite level via one or more indwelling implants 102 at a plurality of sequential times. At 1112, detecting the in vivo CSF compositional information of the biological subject includes collecting in situ CSF compositional information of the biological subject. At 1114, detecting the in vivo CSF compositional information of the biological subject includes collecting in situ CSF spectral information of the biological subject. At 1116, detecting the in vivo CSF compositional information of the biological subject includes collecting a series of serotonin metabolite-related compositional information. At 1118, detecting the in vivo CSF compositional information of the biological subject includes measuring compositional information associated with a CSF serotonin metabolite level. At 1120, detecting the in vivo CSF compositional information of the biological subject includes measuring compositional information associated with a monoamine metabolite level. At 1122, detecting the in vivo CSF compositional information of the biological subject includes measuring compositional information associated with a 5-hydroxyindolacetic acid level.

At 1124, detecting the in vivo CSF compositional information of the biological subject includes detecting the in vivo CSF compositional information of the biological subject at a first time interval, and wherein in situ, real-time, comparing of the detected in vivo CSF compositional information to user-specific compositional model information includes computing the comparison of the detected in vivo CSF compositional information of the biological subject at for the first time interval to the user-specific compositional model information prior to detecting the in vivo CSF compositional information of the biological subject at a second time interval. At 1126, detecting the in vivo CSF compositional information of the biological subject includes detecting a first time series of in vivo CSF compositional information of the biological subject, and wherein in situ, real-time, comparing of the detected in vivo CSF compositional information to user-specific compositional model information includes computing the comparison of the first time series of in vivo CSF compositional information of the biological subject to the user-specific compositional model information prior to detecting a second time series of in vivo CSF compositional information of the biological subject. At 1128, detecting the in vivo CSF compositional information of the biological subject includes detecting a first time series of in vivo CSF compositional information of the biological subject.

At 1130, the method 1100 includes in situ, real-time, comparing of detected in vivo CSF compositional information to user-specific compositional model information. At 1132, in situ, real-time, comparing of the detected in vivo CSF compositional information to user-specific compositional model information includes computing a difference between a detected spectral intensity and a respective user-specific reference spectral intensity. At 1134, in situ, real-time, comparing of the detected in vivo CSF compositional information to user-specific compositional model information includes computing a difference between a detected compositional intensity and a respective user-specific compositional intensity. At 1136, in situ, real-time, comparing of the detected in vivo CSF compositional information to user-specific compositional model information includes performing an in vivo real-time comparison of detected in vivo CSF spectral information to user-specific compositional model information. At 1138, in situ, real-time, comparing of the detected in vivo CSF compositional information to user-specific compositional model information includes in situ, real-time, comparing of detected in vivo CSF compositional information to user-specific compositional model information via a device carried or worn by the biological subject.

At 1140, the method 1100 includes generating a suicidal tendency status. At 1145, the method 1100 includes generating predictive model information based on the detected in vivo CSF compositional information of the biological subject. At 1150, the method 1100 includes updating the user-specific compositional model information based on the generated predict model information prior to in situ, real-time, comparing. At 1155, the method 1100 includes generating predictive model information based on the detected first time series of in vivo CSF compositional information of the biological subject. At 1160, the method 1100 includes in situ, real-time, comparing of the predictive model information to the user-specific compositional model information. At 1165, the method 1100 includes generating a suicidal tendency status in response to in situ, real-time, comparing of the predictive model information to the user-specific compositional model information.

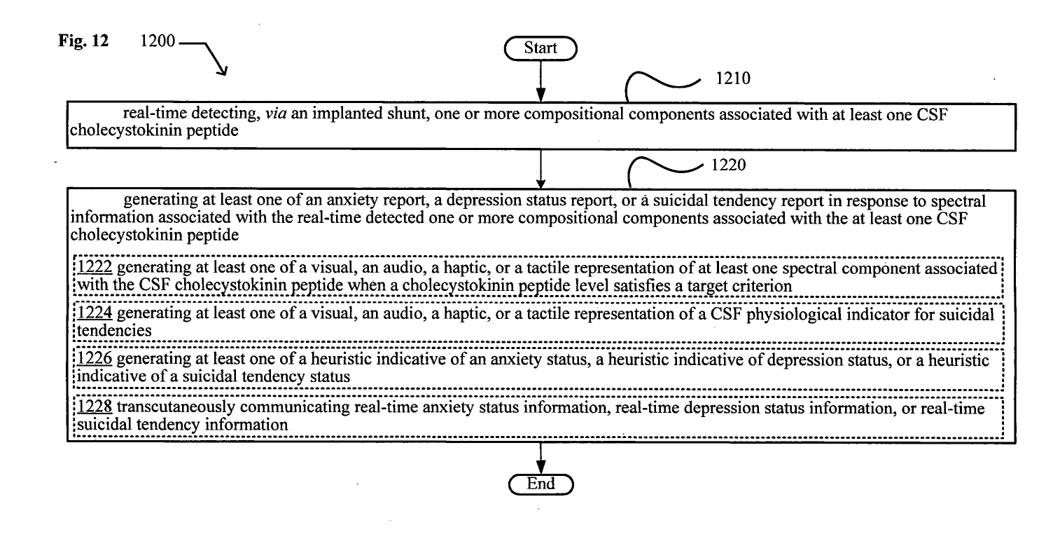

FIG. 12 shows a method 1200 for monitoring a pathological condition associated with a suicidal tendency. At 1210, the method 1200 includes real-time detecting, via an implanted shunt, one or more compositional components associated with at least one CSF cholecystokinin peptide. At 1220, the method 1200 includes generating at least one of an anxiety report, a depression status report, or a suicidal tendency report in response to spectral information associated with the real-time detected one or more compositional components associated with the at least one CSF cholecystokinin peptide. At 1222, generating the at least one of the anxiety report, the depression status report, or the suicidal tendency report includes generating at least one of a visual, an audio, a haptic, or a tactile representation of at least one spectral component associated with the CSF cholecystokinin peptide when a cholecystokinin peptide level satisfies a target criterion. At 1224, generating the at least one of the anxiety report, the depression status report, or the suicidal tendency report includes generating at least one of a visual, an audio, a haptic, or a tactile representation of a CSF physiological indicator for suicidal tendencies. At 1226, generating the at least one of the anxiety report, the depression status report, or the suicidal tendency report includes generating at least one of a heuristic indicative of an anxiety status, a heuristic indicative of depression status, or a heuristic indicative of a suicidal tendency status. At 1228, generating the at least one of the anxiety report, the depression status report, or the suicidal tendency report includes transcutaneously communicating real-time anxiety status information, real-time depression status information, or real-time suicidal tendency information.

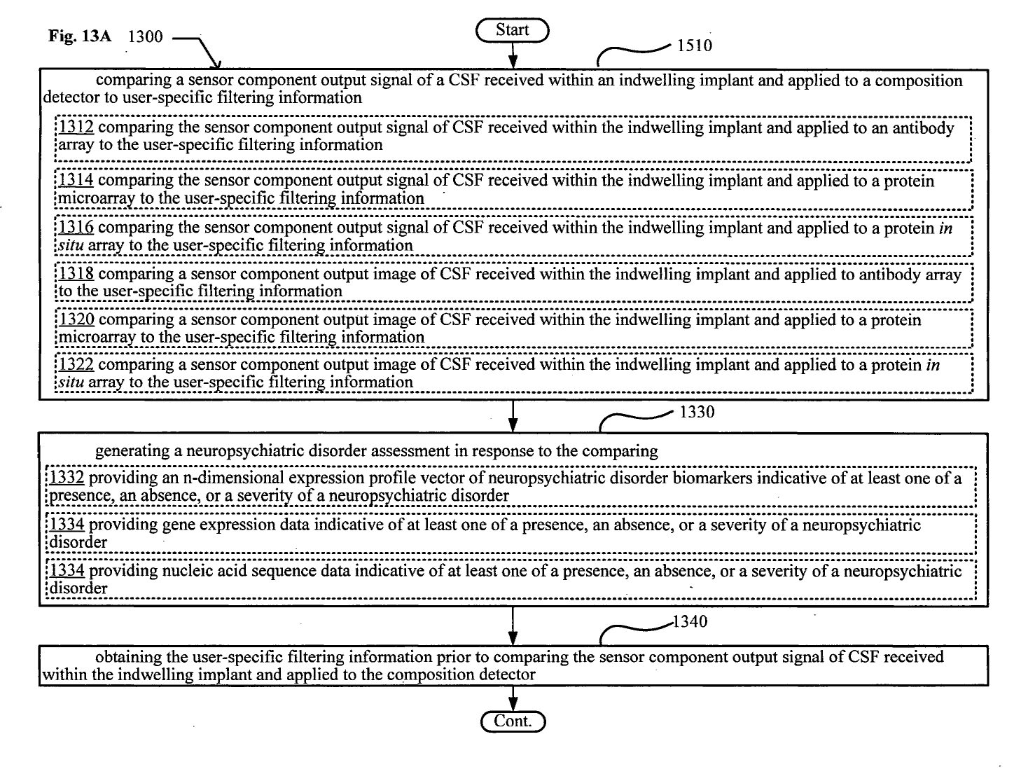

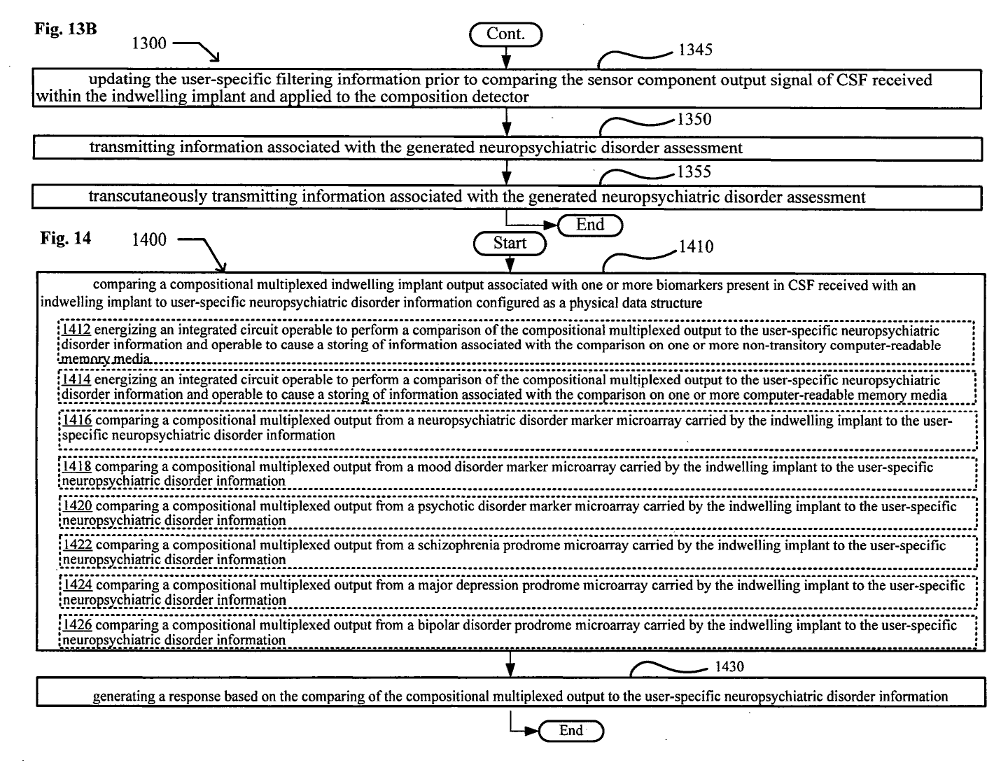

FIGS. 13A and 13B show a method 1300. At 1310, the method 1300 includes comparing a sensor component output signal of CSF received within an indwelling implant 102 and applied to a composition detector to user-specific filtering information 235. At 1312, comparing the sensor component output signal of CSF received within the indwelling implant 102 and applied to the composition detector includes comparing the sensor component output signal of CSF received within the indwelling implant 102 and applied to an antibody array to the user-specific filtering information 235. At 1314, comparing the sensor component output signal of CSF received within the indwelling implant 102 and applied to the composition detector includes comparing the sensor component output signal of CSF received within the indwelling implant 102 and applied to a protein microarray to the user-specific filtering information 235. At 1316, comparing the sensor component output signal of CSF received within the indwelling implant 102 and applied to the composition detector includes comparing the sensor component output signal of CSF received within the indwelling implant 102 and applied to a protein in situ array to the user-specific filtering information 235.

At 1318, comparing the sensor component output signal of CSF received within the indwelling implant 102 and applied to the composition detector includes comparing a sensor component output image of CSF received within the indwelling implant 102 and applied to an antibody array to the user-specific filtering information 235. At 1320, comparing the sensor component output signal of CSF received within the indwelling implant 102 and applied to the composition detector includes comparing a sensor component output image of CSF received within the indwelling implant 102 and applied to a protein microarray to the user-specific filtering information 235. At 1322, comparing the sensor component output signal of CSF received within the indwelling implant 102 and applied to the composition detector includes comparing a sensor component output image of CSF received within the indwelling implant 102 and applied to a protein in situ array to the user-specific filtering information 235.

At 1330, the method 1300 includes generating a neuropsychiatric disorder assessment in response to the comparison. At 1332, generating the neuropsychiatric disorder assessment includes providing an n-dimensional expression profile vector of neuropsychiatric disorder biomarkers indicative of at least one of a presence, an absence, or a severity of a neuropsychiatric disorder. At 1334, generating the neuropsychiatric disorder assessment includes providing gene expression data indicative of at least one of a presence, an absence, or a severity of a neuropsychiatric disorder. At 1336, generating the neuropsychiatric disorder assessment includes providing nucleic acid sequence data indicative of at least one of a presence, an absence, or a severity of a neuropsychiatric disorder.

At 1340, the method 1300 includes obtaining the user-specific filtering information 235 prior to comparing the sensor component output signal of CSF received within the indwelling implant 102 and applied to the composition detector. At 1345, the method 1300 includes updating the user-specific filtering information 235 prior to comparing the sensor component output signal of CSF received within the indwelling implant 102 and applied to the composition detector. At 1350, the method 1300 includes transmitting information associated with the generated neuropsychiatric disorder assessment. At 1355, the method 1300 includes transcutaneously transmitting information associated with the generated neuropsychiatric disorder assessment.

FIG. 14 shows an in vivo method 1400 for real-time monitoring of one or more biomarkers within CSF. At 1410, the method 1400 includes comparing a compositional multiplexed indwelling implant 102 output associated with one or more biomarkers present in CSF received with an indwelling implant 102 to user-specific neuropsychiatric disorder information configured as a physical data structure 230. At 1412, comparing the compositional multiplexed output includes energizing an integrated circuit operable to perform a comparison of the compositional multiplexed output to the user-specific neuropsychiatric disorder information and operable to cause a storing of information associated with the comparison on one or more non-transitory computer-readable memory media 215. At 1414, comparing the compositional multiplexed output includes energizing an integrated circuit operable to perform a comparison of the compositional multiplexed output to the user-specific neuropsychiatric disorder information and operable to cause a storing of information associated with the comparison on one or more computer-readable memory media 215. At 1416, comparing the compositional multiplexed output includes comparing a compositional multiplexed output from a neuropsychiatric disorder marker microarray carried by the indwelling implant 102 to the user-specific neuropsychiatric disorder information.

At 1418, comparing the compositional multiplexed output includes comparing a compositional multiplexed output from a mood disorder marker microarray carried by the indwelling implant 102 to the user-specific neuropsychiatric disorder information. At 1420, comparing the compositional multiplexed output includes comparing a compositional multiplexed output from a psychotic disorder marker microarray carried by the indwelling implant 102 to the user-specific neuropsychiatric disorder information. At 1422, comparing the compositional multiplexed output includes comparing a compositional multiplexed output from a schizophrenia prodrome microarray carried by the indwelling implant 102 to the user-specific neuropsychiatric disorder information. At 1424, comparing the compositional multiplexed output includes comparing a compositional multiplexed output from a major depression prodrome microarray carried by the indwelling implant 102 to the user-specific neuropsychiatric disorder information. At 1426, comparing the compositional multiplexed output includes comparing a compositional multiplexed output from a bipolar disorder prodrome microarray carried by the indwelling implant 102 to the user-specific neuropsychiatric disorder information. At 1430, the method 1400 includes generating a response based on the comparing of compositional multiplexed output to the user-specific neuropsychiatric disorder information.

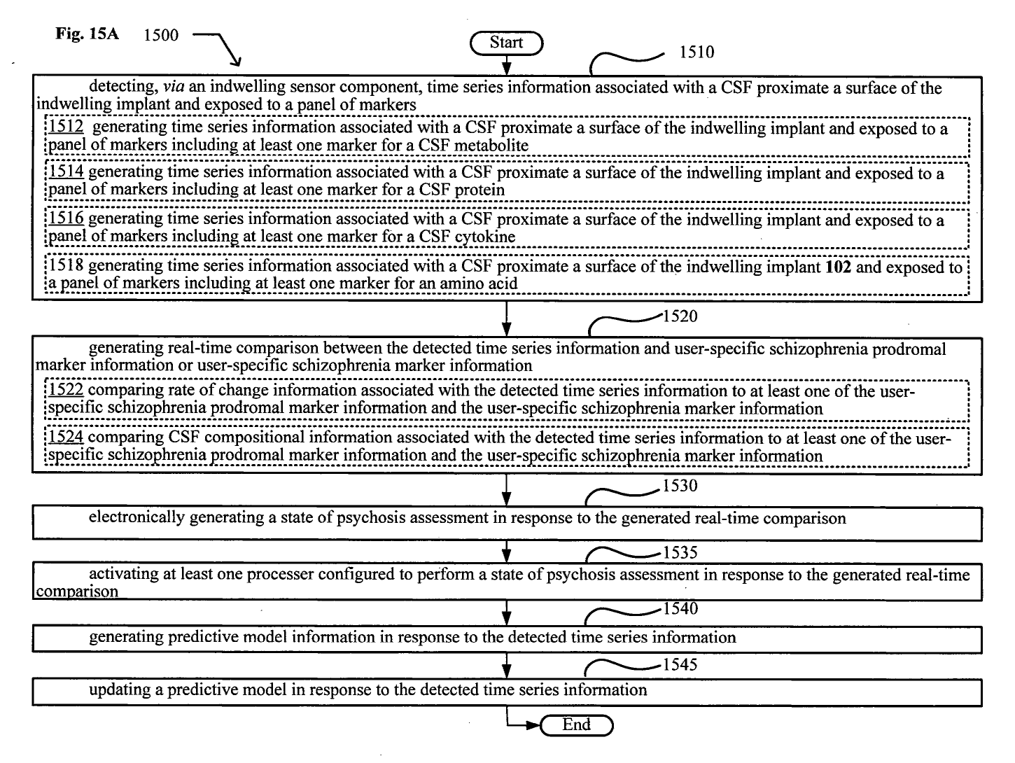

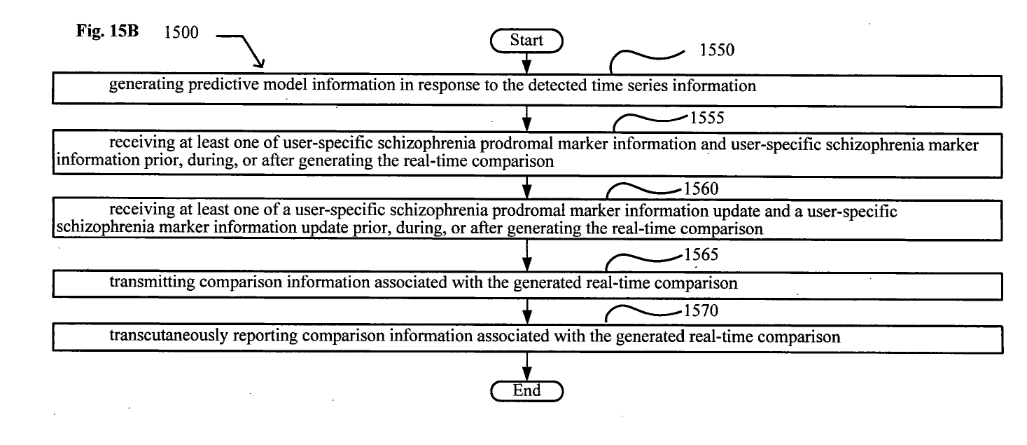

FIGS. 15A and 15B show a method 1500 for diagnosing schizophrenia. At 1510, the method 1500 includes detecting, via an indwelling sensor component 204, time series information associated with CSF proximate a surface of the indwelling implant 102 and exposed to a panel of markers. At 1512, detecting, via the indwelling sensor component 204, the time series information includes generating time series information associated with CSF proximate a surface of the indwelling implant 102 and exposed to a panel of markers including at least one marker for a CSF metabolite. At 1514, detecting, via the indwelling sensor component 204, the time series information includes generating time series information associated with CSF proximate a surface of the indwelling implant 102 and exposed to a panel of markers including at least one marker for a CSF protein. At 1516, detecting, via the indwelling sensor component 204, the time series information includes generating time series information associated with CSF proximate a surface of the indwelling implant 102 and exposed to a panel of markers including at least one marker for a CSF cytokine. At 1518, detecting, via the indwelling sensor component 204, the time series information includes generating time series information associated with CSF proximate a surface of the indwelling implant 102 and exposed to a panel of markers including at least one marker for an amino acid.

At 1520, the method 1500 includes generating real-time comparison between the detected time series information and user-specific schizophrenia prodromal marker information or user-specific schizophrenia marker information. At 1522, generating the real-time comparison includes comparing rate of change information associated with the detected time series information to at least one of the user-specific schizophrenia prodromal marker information and the user-specific schizophrenia marker information. At 1524, generating the real-time comparison includes comparing CSF compositional information associated with the detected time series information to at least one of the user-specific schizophrenia prodromal marker information and the user-specific schizophrenia marker information.

At 1530, the method 1500 includes electronically generating a state of psychosis assessment in response to the generated real-time comparison. At 1535, the method 1500 includes activating at least one processer configured to perform a state of psychosis assessment in response to the generated real-time comparison. At 1540, the method 1500 includes generating predictive model information in response to the detected time series information. At 1545, the method 1500 includes updating a predictive model in response to the detected time series information.

At 1550, the method 1500 includes generating predictive model information in response to the detected time series information. In an embodiment, enerating the real-time comparison between the detected time series information and user-specific schizophrenia prodromal marker information or the user-specific schizophrenia marker information includes generating a real-time comparison between the detected time series information and the generated predictive model information. At 1555, the method 1500 includes receiving at least one of user-specific schizophrenia prodromal marker information and user-specific schizophrenia marker information prior, during, or after generating the real-time comparison. At 1560, the method 1500 includes receiving at least one of a user-specific schizophrenia prodromal marker information update and a user-specific schizophrenia marker information update prior, during, or after generating the real-time comparison. At 1565, the method 1500 includes transmitting comparison information associated with the generated real-time comparison. At 1570, the method 1500 includes transcutaneously reporting comparison information associated with the generated real-time comparison.

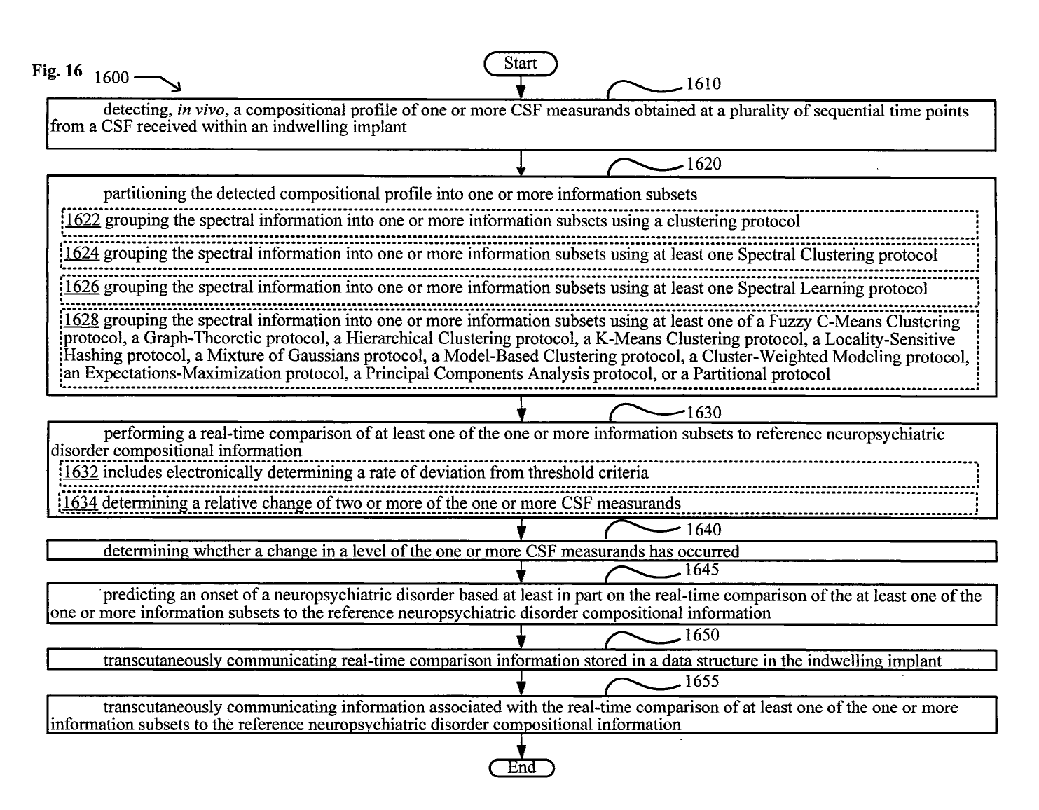

FIG. 16 shows a method 1600. At 1610, the method 1600 includes detecting, in vivo, a compositional profile of one or more CSF measurands obtained at a plurality of sequential time points from a CSF received within an indwelling implant 102. At 1620, the method 1600 includes partitioning the detected compositional profile into one or more information subsets. At 1622, partitioning the detected compositional profile into the one or more information subsets includes grouping the spectral information into one or more information subsets using a clustering protocol. At 1624, partitioning the detected compositional profile into the one or more information subsets includes grouping the spectral information into one or more information subsets using at least one Spectral Clustering protocol.

At 1626, partitioning the detected compositional profile into the one or more information subsets includes grouping the spectral information into one or more information subsets using at least one Spectral Learning protocol. At 1628, partitioning the detected compositional profile into the one or more information subsets includes grouping the spectral information into one or more information subsets using at least one of a Fuzzy C-Means Clustering protocol; a Graph-Theoretic protocol, a Hierarchical Clustering protocol, a K-Means Clustering protocol, a Locality-Sensitive Hashing protocol, a Mixture of Gaussians protocol, a Model-Based Clustering protocol, a Cluster-Weighted Modeling protocol, an Expectations-Maximization protocol, a Principal Components Analysis protocol, or a Partitional protocol.

At 1630, the method 1600 includes performing a real-time comparison of at least one of the one or more information subsets to reference neuropsychiatric disorder compositional information. At 1632, performing the real-time comparison includes electronically determining a rate of deviation from threshold criteria. At 1634, performing the real-time comparison includes determining a relative change of two or more of the one or more CSF measurands. At 1640, the method 1600 includes determining whether a change in a level of the one or more CSF measurands has occurred. At 1645, the method 1600 includes predicting an onset of a neuropsychiatric disorder based at least in part on the real-time comparison of the at least one of the one or more information subsets to the reference neuropsychiatric disorder compositional information. At 1650, the method 1600 includes transcutaneously communicating real-time comparison information stored in a data structure 230 in the indwelling implant 102. At 1655, the method 1600 includes transcutaneously communicating information associated with the real-time comparison of at least one of the one or more information subsets to the reference neuropsychiatric disorder compositional information.

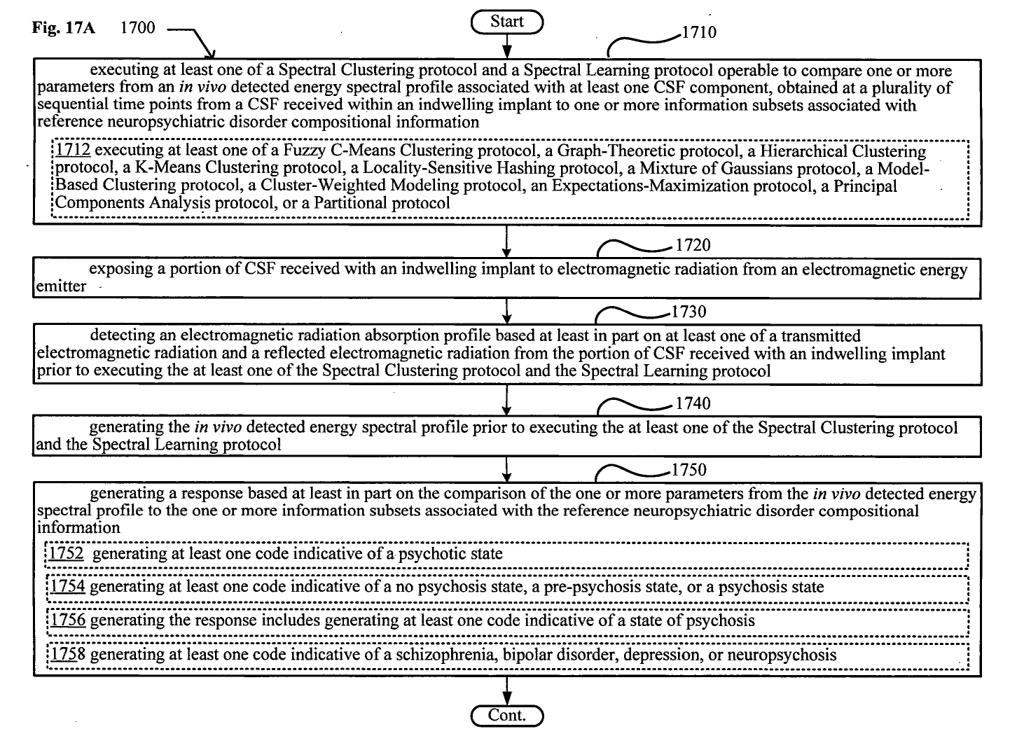

FIGS. 17A and 17B show a method 1700. At 1710, the method 1700 includes executing at least one of a Spectral Clustering protocol and a Spectral Learning protocol operable to compare one or more parameters from an in vivo detected energy spectral profile associated with at least one CSF component, obtained at a plurality of sequential time points from CSF received within an indwelling implant 102, to one or more information subsets associated with reference neuropsychiatric disorder compositional information. At 1712, executing the at least one of the Spectral Clustering protocol and the Spectral Learning protocol includes executing at least one of a Fuzzy C-Means Clustering protocol, a Graph-Theoretic protocol, a Hierarchical Clustering protocol, a K-Means Clustering protocol, a Locality-Sensitive Hashing protocol, a Mixture of Gaussians protocol, a Model-Based Clustering protocol, a Cluster-Weighted Modeling protocol, an Expectations-Maximization protocol, a Principal Components Analysis protocol, or a Partitional protocol.

At 1720, the method 1700 includes exposing a portion of CSF received with an indwelling implant 102 to electromagnetic radiation from an electromagnetic energy emitter. At 1730, the method 1700 includes detecting an electromagnetic radiation absorption profile based at least in part on at least one of a transmitted electromagnetic radiation and a reflected electromagnetic radiation from the portion of CSF received with an indwelling implant 102 prior to executing the at least one of the Spectral Clustering protocol and the Spectral Learning protocol.

At 1740, the method 1700 includes generating the in vivo detected energy spectral profile prior to executing the at least one of the Spectral Clustering protocol and the Spectral Learning protocol. At 1750, the method 1700 includes generating a response based at least in part on the comparison of the one or more parameters from the in vivo detected energy spectral profile to the one or more information subsets associated with the reference neuropsychiatric disorder compositional information.

At 1752, generating the response includes generating at least one code indicative of a psychotic state. At 1754, generating the response includes generating at least one code indicative of a no psychosis state, a pre-psychosis state, or a psychosis state. At 1756, generating the response includes generating at least one code indicative of a state of psychosis. At 1758, generating the response includes generating at least one code indicative of a schizophrenia, bipolar disorder, depression, or neuropsychosis. At 1760, generating the response includes generating at least one code indicative of a neuropsychiatric disorder assessment. At 1762, generating the response includes generating at least one code indicative of a prodromal state of psychosis or a first-onset state of psychosis. At 1764, generating the response includes generating at least one code indicative of a predisposition to suicide.

At 1766, generating the response includes generating a response based on a comparison of one or more parameters from the in vivo detected energy spectral profile to a threshold diameter of at least one cluster associated with a set of reference cluster information associated with the reference neuropsychiatric disorder compositional information. At 1768, generating the response includes generating a response based on a comparison of the one or more parameters from the in vivo detected energy spectral profile to an average squared distance of at least one cluster centroid associated with the reference neuropsychiatric disorder compositional information. At 1770, generating the response includes generating a response based on a comparison of the one or more parameters from the in vivo detected energy spectral profile to an inverse of a distance to at least one cluster centroid associated with the reference neuropsychiatric disorder compositional information. At 1772, generating the response includes updating at least one parameter associated with the statistical learning model in response to a comparison of the one or more parameters from the in vivo detected energy spectral profile to the reference neuropsychiatric disorder compositional information. At 1780, the method 1700 includes predicting an onset of a neuropsychiatric disorder based on a comparison of the one or more parameters from the in vivo detected energy spectral profile to the one or more information subsets associated with the reference neuropsychiatric disorder compositional information. At 1785, the method 1700 includes predicting a time to onset of a neuropsychiatric disorder based on a comparison of the one or more parameters from the in vivo detected energy spectral profile to the one or more information subsets associated with the reference neuropsychiatric disorder compositional information.

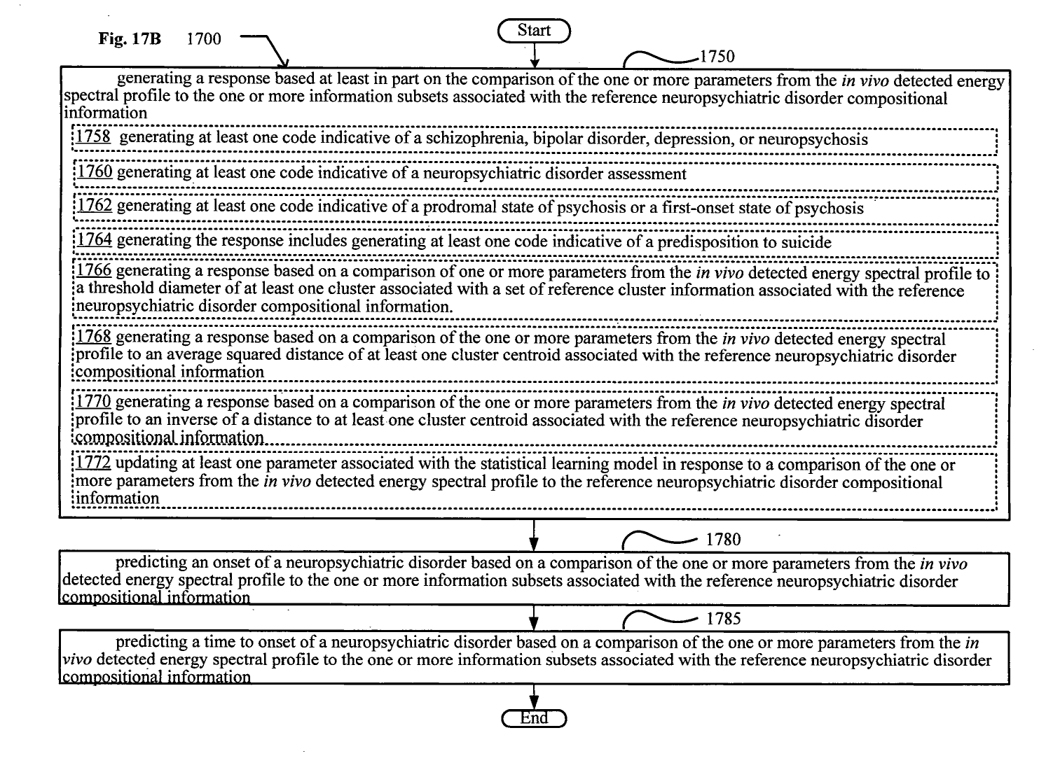

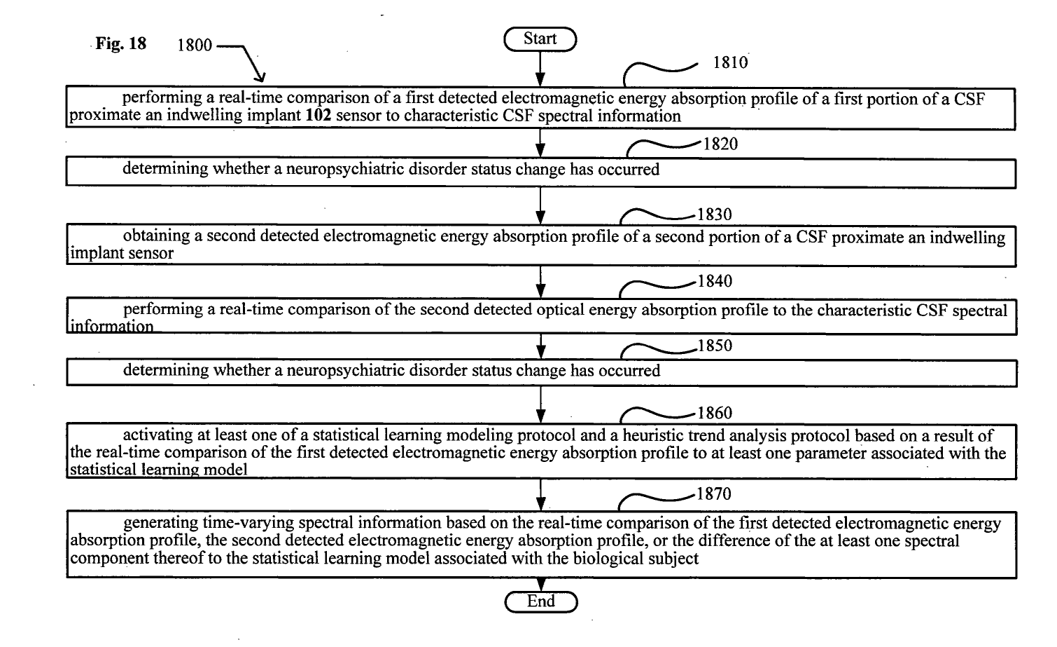

FIG. 18 shows a method 1800. At 1810, the method 1800 includes performing a real-time comparison of a first detected electromagnetic energy absorption profile of a first portion of a CSF proximate an indwelling implant 102 sensor to characteristic CSF spectral information. At 1820, the method 1800 includes determining whether a neuropsychiatric disorder status change has occurred. At 1830, the method 1800 includes obtaining a second detected electromagnetic energy absorption profile of a second portion of a CSF proximate an indwelling implant sensor. At 1840, the method 1800 includes performing a real-time comparison of the second detected optical energy absorption profile to the characteristic CSF spectral information. At 1850, the method 1800 includes determining whether a neuropsychiatric disorder status change has occurred. At 1860, the method 1800 includes activating at least one of a statistical learning modeling protocol and a heuristic trend analysis protocol based on a result of the real-time comparison of the first detected electromagnetic energy absorption profile to at least one parameter associated with the statistical learning model. At 1870, the method 1800 includes generating time-varying spectral information based on the real-time comparison of the first detected electromagnetic energy absorption profile, the second detected electromagnetic energy absorption profile, or the difference of the at least one spectral component thereof to the statistical learning model associated with the biological subject.

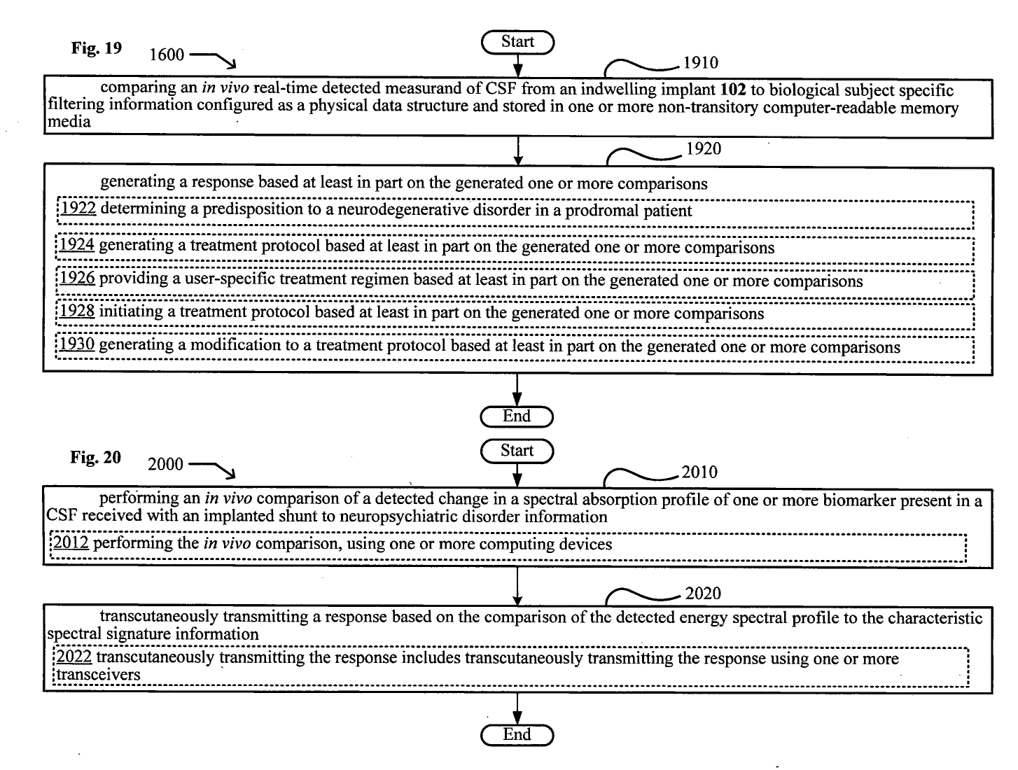

FIG. 19 shows a method 1900. At 1910, the method 1900 includes comparing an in vivo real-time detected measurand of CSF from an indwelling implant 102 to biological subject specific filtering information configured as a physical data structure 230 and stored in one or more non-transitory computer-readable memory media 215. At 1920, the method 1900 includes generating a response based at least in part on the generated one or more comparisons. At 1922, generating the response includes determining a predisposition to a neurodegenerative disorder in a prodromal patient. At 1924, generating the response includes generating a treatment protocol based at least in part on the generated one or more comparisons. At 1926, generating the response includes providing a user-specific treatment regimen based at least in part on the generated one or more comparisons. At 1928, generating the response includes initiating a treatment protocol based at least in part on the generated one or more comparisons. At 1930, generating the response includes generating a modification to a treatment protocol based at least in part on the generated one or more comparisons.

FIG. 20 shows an in vivo method 2000 for real-time monitoring of one or more biomarkers within CSF. At 2010, the method 2000 includes performing an in vivo comparison of a detected change in a spectral absorption profile of one or more biomarkers present in a CSF received with an implanted shunt to neuropsychiatric disorder information. At 2012, performing the in vivo comparison includes performing the in vivo comparison, using one or more computing devices. At 2020, the method 2000 includes transcutaneously transmitting a response based on the comparison of the detected energy spectral profile to the characteristic spectral signature information. At 2022, transcutaneously transmitting the response includes transcutaneously transmitting the response using one or more transceivers 207.

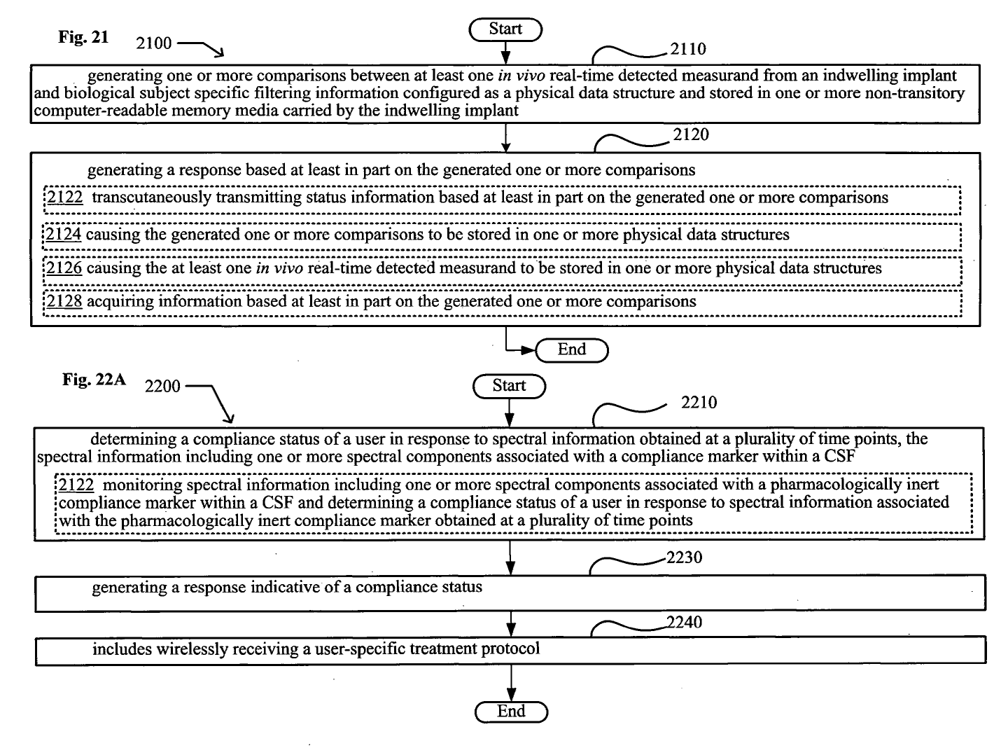

FIG. 21 shows a monitoring method 2100.

At 2110, the method 2100 includes generating one or more comparisons between at least one in vivo real-time detected measurand from an indwelling implant 102 and biological subject specific filtering information configured as a physical data structure 230 and stored in one or more non-transitory computer-readable memory media 215 carried by the indwelling implant 102. At 2120, the method 2100 includes generating a response based at least in part on the generated one or more comparisons. At 2122, generating the response includes transcutaneously transmitting status information based at least in part on the generated one or more comparisons. At 2124, generating the response includes causing the generated one or more comparisons to be stored in one or more physical data structures 230. At 2126, generating the response includes causing the at least one in vivo real-time detected measurand to be stored in one or more physical data structures 230. At 2128, generating the response includes acquiring information based at least in part on the generated one or more comparisons.

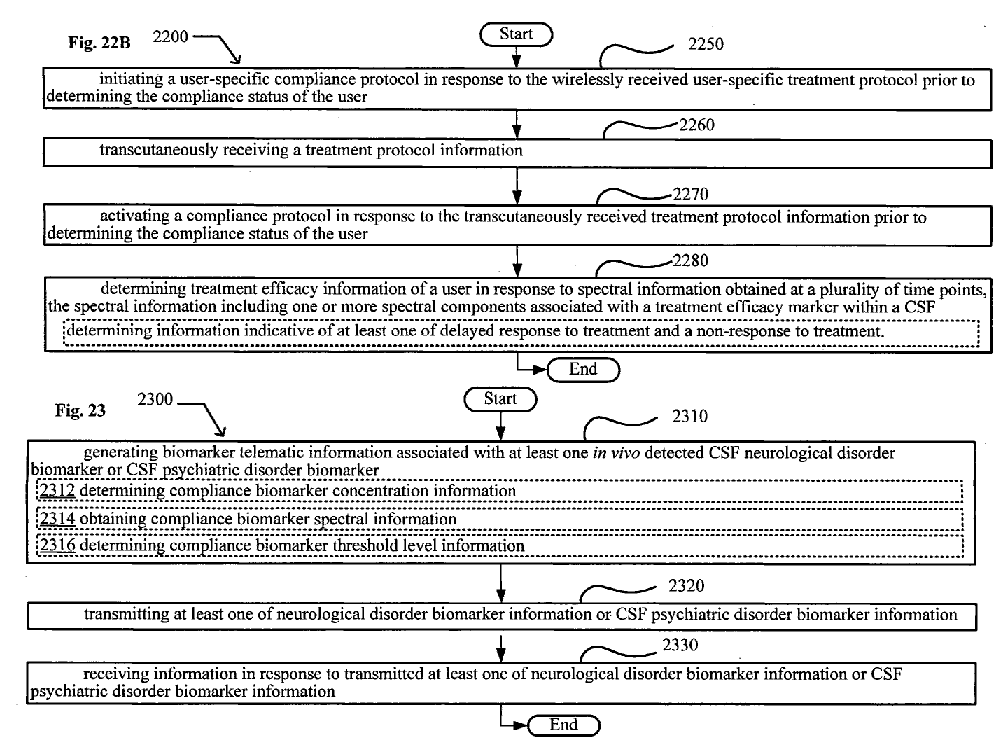

FIGS. 22A and 22B show a real-time in vivo method 2200 of assessing a treatment efficacy or a treatment compliance associated with an acute or a chronic neuropsychiatric condition.

At 2210, the method 2200 includes determining a compliance status of a user in response to spectral information obtained at a plurality of time points, the spectral information including one or more spectral components associated with a compliance marker within a CSF. At 2212, determining a compliance status includes monitoring spectral information including one or more spectral components associated with a pharmacologically inert compliance marker within a CSF and determining a compliance status of a user in response to spectral information associated with the pharmacologically inert compliance marker obtained at a plurality of time points. At 2230, the method 2200 includes generating a response indicative of a compliance status. At 2240, the method 2200 includes wirelessly receiving a user-specific treatment protocol. At 2250, the method 2200 includes initiating a user-specific compliance protocol in response to the wirelessly received user-specific treatment protocol prior to determining the compliance status of the user. At 2260, the method 2200 includes transcutaneously receiving a treatment protocol information. At 2270, the method 2200 includes activating a compliance protocol in response to the transcutaneously received treatment protocol information prior to determining the compliance status of the user. At 2280, the method 2200 includes determining treatment efficacy information of a user in response to spectral information obtained at a plurality of time points, the spectral information including one or more spectral components associated with a treatment efficacy marker within a CSF. At 2282, determining the treatment efficacy information includes determining information indicative of at least one of a delayed response to treatment and a non-response to treatment.

FIG. 23 shows a telematic monitoring method 2300. At 2310, the method 2000 includes generating biomarker telematic information associated with at least one in vivo detected CSF neurological disorder biomarker or CSF psychiatric disorder biomarker. At 2312, generating the biomarker telematic information includes determining compliance biomarker concentration information. At 2314, generating the biomarker telematic information includes obtaining compliance biomarker spectral information. At 2316, generating the biomarker telematic information includes determining compliance biomarker threshold level information. At 2320, the method 2000 includes transmitting at least one of neurological disorder biomarker information or CSF psychiatric disorder biomarker information. At 2330, the method 2000 includes receiving information in response to transmitted at least one of neurological disorder biomarker information or CSF psychiatric disorder biomarker information.

1.-353. (canceled)

354. An implantable wireless biotelemetry device, comprising:
a sensor component configured to detect at least one biomarker profile of a cerebrospinal fluid received within one or more fluid-flow passageways of the implantable wireless biotelemetry device;
one or more computer-readable memory media including executable instructions stored thereon that, when executed on a computer, instruct a computing device to
retrieve from storage one or more parameters associated with reference cerebrospinal fluid biomarker spectral information associated with at least one neuropsychiatric disorder, and to
perform a comparison of a detected at least one biomarker profile to the retrieved one or more parameters; and
a transceiver operable to concurrently or sequentially transmit or receive information in response to the comparison.

355. (canceled)

356. The implantable wireless biotelemetry device of claim 354, wherein the transceiver is configured to report status information at a plurality of time intervals and to enter a receive mode for a period after transmitting the report information.

357. The implantable wireless biotelemetry device of claim 354, wherein the transceiver is configured to request cerebrospinal fluid biomarker spectral information in response to the comparison.

358. (canceled)

359. The implantable wireless biotelemetry device of claim 354, wherein the one or more computer-readable memory media further include executable instructions stored thereon that, when executed on a computer, instruct a computing device to generate a schizophrenia spectrum diagnosis in response to the comparison.

360. The implantable wireless biotelemetry device of claim 354, wherein the one or more computer-readable memory media further include executable instructions stored thereon that, when executed on a computer, instruct a computing device to cause the storing of neurobiological change information in response to the comparison.

361. The implantable wireless biotelemetry device of claim 354, wherein the one or more computer-readable memory media further include executable instructions stored thereon that, when executed on a computer, instruct a computing device to generate an affective disorder score in response to the comparison.

362. The implantable wireless biotelemetry device of claim 354, wherein the reference cerebrospinal fluid biomarker spectral information associated with the at least one neuropsychiatric disorder includes cerebrospinal fluid psychiatric disorder biomarker spectral information.

363. (canceled)

364. The implantable wireless biotelemetry device of claim 354, wherein the reference cerebrospinal fluid biomarker spectral information associated with the at least one neuropsychiatric disorder includes cerebrospinal fluid mood disorder biomarker spectral information.

365. The implantable wireless biotelemetry device of claim 354, wherein the reference cerebrospinal fluid biomarker spectral information associated with the at least one neuropsychiatric disorder includes cerebrospinal fluid chronic dementia disease biomarker spectral information.

366. A method, comprising:
comparing an in vivo real-time detected measurand of cerebrospinal fluid from an indwelling implant to biological subject specific mental disorder filtering information configured as a physical data structure and stored in one or more non-transitory computer-readable memory media; and
generating a response based at least in part on the generated one or more comparisons.

367. The method of claim 366, wherein generating the response includes determining a predisposition to a neurodegenerative disorder in a prodromal patient.

368. (canceled)

369. The method of claim 366, wherein generating the response includes providing a user-specific treatment regimen based at least in part on the generated one or more comparisons.

370. (canceled)

371. The method of claim 366, wherein generating the response includes generating a modification to a treatment protocol based at least in part on the generated one or more comparisons.

372. An in vivo method for real-time monitoring of one or more biomarkers within a cerebrospinal fluid, comprising:
performing an in vivo comparison of a detected change in a spectral absorption profile of one or more biomarkers present in a cerebrospinal fluid received with an implanted shunt to neuropsychiatric disorder information; and
transcutaneously transmitting a response based on the comparison of the detected energy spectral profile to the characteristic spectral signature information.

373. (canceled)

374. The in vivo method for real-time monitoring of one or more biomarkers within a cerebrospinal fluid of claim 372, wherein transcutaneously transmitting the response includes transcutaneously transmitting the response using one or more transceivers.

375. A monitoring method, comprising:
generating one or more comparisons between at least one in vivo real-time detected measurand from an indwelling implant and biological subject specific mental disorder filtering information configured as a physical data structure and stored in one or more non-transitory computer-readable memory media carried by the indwelling implant; and
generating a response based at least in part on the generated one or more comparisons.

376. The method of claim 375, wherein generating the response includes transcutaneously transmitting status information based at least in part on the generated one or more comparisons.

377. The method of claim 375, wherein generating the response includes causing the generated one or more comparisons to be stored in one or more physical data structures.

378. (canceled)

379. The method of claim 375, wherein generating the response includes acquiring information based at least in part on the generated one or more comparisons.

380.-391. (canceled)
